(19) United States
(12) Patent Application Publication
(10) Pub. No.: US 2018/0372751 A1
(43) Pub. Date: Dec. 27, 2018

(54) LYSINE REACTIVE PROBES AND USES THEREOF

(71) Applicant: The Scripps Research Institute, La Jolla, CA (US)

(72) Inventors: Benjamin F. CRAVATT, La Jolla, CA (US); Stephan M. HACKER, San Diego, CA (US); Keriann M. BACKUS, La Jolla, CA (US)

(21) Appl. No.: 16/016,392

(22) Filed: Jun. 22, 2018

Related U.S. Application Data
(60) Provisional application No. 62/524,383, filed on Jun. 23, 2017.

(51) Int. Cl. G01N 33/68; C12N 9/10; G01N 33/58; G01N 33/50; C12N 9/64

(52) U.S. Cl. CPC G01N 33/6812; C12N 9/1007; G01N 33/582; G01N 33/5008; C12N 9/6472; C12Y 304/22063; G01N 33/6842; C12Y 101/01042; C12Y 201/01023; C12Y 304/22061; C12N 9/641

(57) ABSTRACT

Disclosed herein are methods and compounds for profiling a lysine reactive protein. Also described herein are methods, compounds, and compositions for identifying a small molecule fragment ligand that interacts with a reactive lysine residue.

### CROSS-REFERENCE

This application claims the benefit of U.S. Provisional Application No. 62/524,383, filed on Jun. 23, 2017, which is incorporated herein by reference in its entirety.

### STATEMENT AS TO FEDERALLY SPONSORED RESEARCH

The invention disclosed herein was made, at least in part, with U.S. government support under Grant Nos. CA087660, CA132630, GM108208, and GM069832 by the National Institutes of Health. Accordingly, the U.S. Government has certain rights in this invention.

### INCORPORATION BY REFERENCE OF TABLE SUBMITTED AS TEXT FILE VIA EFS-WEB

The instant application contains Tables 2 and 3, which have been submitted as a computer readable text file in ASCII format via EFS-Web and are hereby incorporated in their entireties by reference herein. The text files, created date of Jun. 14, 2018, are named 48054-708-201Table2.txt and 48054-708-201Table3.txt, respectively, and are 473 and 1596 kb in size.

### BACKGROUND OF THE DISCLOSURE

Protein function assignment has been benefited from genetic methods, such as target gene disruption, RNA interference, and genome editing technologies, which selectively disrupt the expression of proteins in native biological systems. Chemical probes offer a complementary way to perturb proteins that have the advantages of producing graded (dose-dependent) gain-(agonism) or loss-(antagonism) of-function effects that are introduced acutely and reversibly in cells and organisms. Small molecules present an alternative method to selectively modulate proteins and to serve as leads for the development of novel therapeutics.

### SUMMARY OF THE DISCLOSURE

Disclosed herein, in certain embodiments, is a method of identifying a reactive lysine of a protein, comprising: (a) providing a protein sample comprising isolated proteins, living cells, or a cell lysate; (b) contacting the protein sample with a probe compound of Formula (I) at a first concentration for a time sufficient for the probe compound to react with the reactive lysine of the protein sample; and (c) analyzing the proteins of the protein sample to identify the reactive lysine that bound with the probe compound at the first concentration; wherein the probe compound has a structure represented by Formula (I):

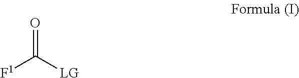

wherein F¹ is a small molecule fragment moiety comprising an alkyne moiety, a fluorophore moiety, a labeling group, or a combination thereof; and LG is a leaving group moiety. In some embodiments, F¹ comprises an alkyne moiety. In some embodiments, F¹ comprises a fluorophore moiety. In some embodiments, LG comprises a succinimide moiety or a phenyl moiety. In some embodiments, LG comprises the phenyl moiety. In some embodiments, the phenyl moiety comprises one or more substituents selected from the group consisting of halogen, C₁-C₆fluoroalkyl, —CN, —NO₂, —S(═O)R¹, —S(═O)₂R¹, —S(═O)₂OM, —N(R¹)S(═O)₂R¹, —S(═O)₂NR¹R², —C(═O)R¹, —C(═O)OM, —OC(═O)R¹, —C(═O)OR², —OC(═O)OR², —C(═O)NR¹R², —OC(═O)NR¹R², —NR C(═O)NR R², and —NR¹C(═O)R¹; each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl; R² is independently selected from the group consisting of H, D, C₁-C₆alkyl, C₁-C₆fluoroalkyl, C₁-C₆heteroalkyl, and a substituted or unsubstituted aryl; or R¹ and R⁶ are taken together with the intervening atoms joining R⁵ and R⁶ to form a 5- or 6-membered ring; and M is Li, Na, K, or —N(R²)₄. In some embodiments, the probe compound has a structure selected from:

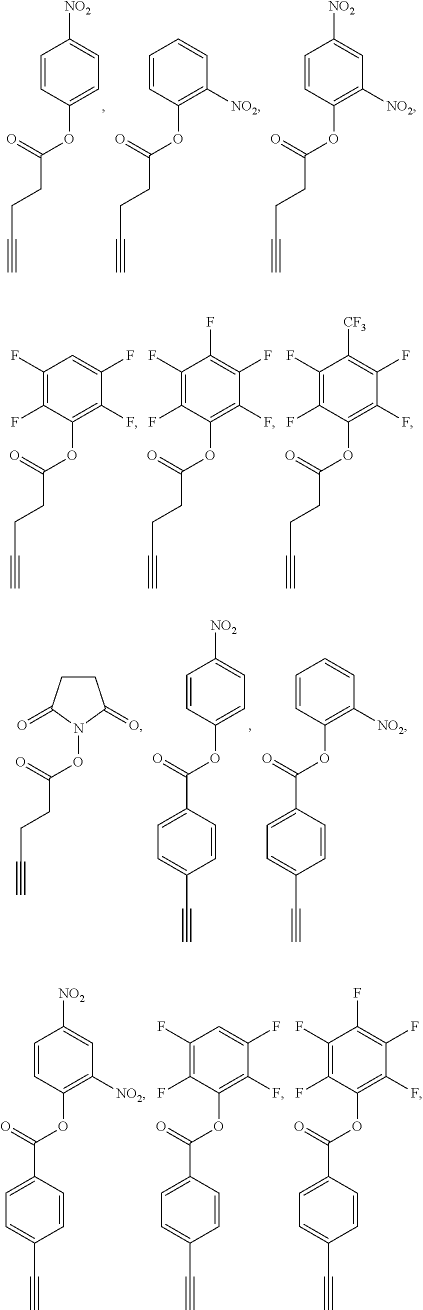

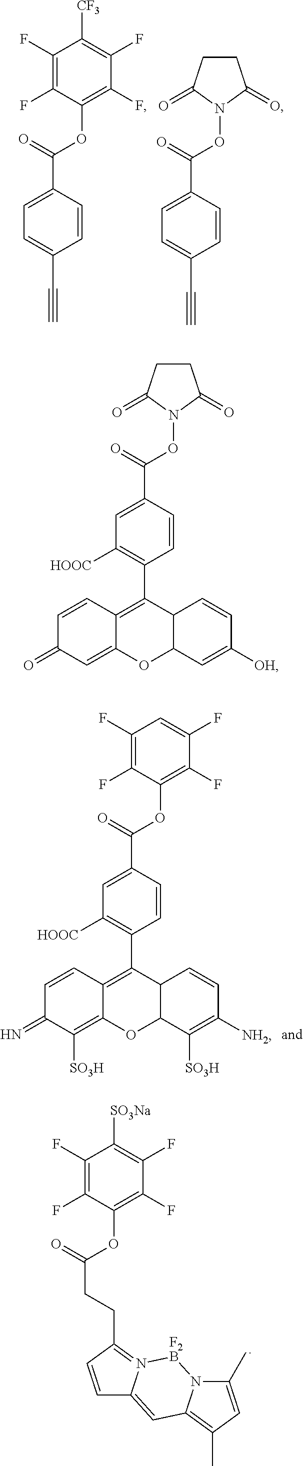

In some embodiments, the analyzing of step (c) further comprises tagging at least one lysine-containing protein-ligand complex of step (b) to generate a tagged lysine-containing protein-ligand complex. In some embodiments, the analyzing of step (c) further comprises isolating the tagged lysine-containing protein-ligand complex. In some embodiments, the tagging comprises a biotin moiety. In some embodiments, the biotin moiety comprises biotin or a biotin derivative. In some embodiments, the biotin derivative comprises desthiobiotin, biotin alkyne or biotin azide. In some embodiments, the biotin moiety comprises desthiobiotin. In some embodiments, the method further comprises (a) providing an protein sample comprising isolated proteins, living cells, or a cell lysate and separating the protein sample into a first protein sample and a second protein sample; (b) contacting the first protein sample with a probe compound of Formula (I) at a first concentration for a time sufficient for the probe compound to react with a reactive lysine of the first protein sample, and contacting the second protein sample with the probe compound of Formula (I) at a second concentration for a sufficient time for the probe compound to react with a reactive lysine of the second protein sample; (c) tagging the proteins of the first protein sample and the second protein sample of step b) to generate tagged proteins; and (d) isolating the tagged the proteins of the first protein sample and the second protein sample for analysis.

Disclosed herein, in certain embodiments, is a method of identifying a reactive lysine of a protein, comprising: (a) providing a protein sample comprising isolated proteins, living cells, or a cell lysate and separating the protein sample into a first protein sample and a second protein sample; (b) contacting the first protein sample with a probe compound of Formula I at a first concentration for a time sufficient for the probe compound to react with a reactive lysine of the first protein sample, and contacting the second protein sample with the probe compound of Formula (I) at a second concentration for a sufficient time for the probe compound to react with a reactive lysine of the second protein sample; (c) analyzing the proteins of the first protein sample and the second protein samples of step b) to identify the reactive lysines that bound with the probe compound; (d) comparing the identity of the reactive lysines of step c) from the first protein sample at the first concentration of probe compound to the reactive lysines from the second protein sample at the second concentration of probe compound; and (e) based on step d), determining a reactive lysine of a protein; wherein the probe compound has a structure represented by Formula (I):

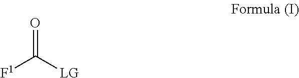

wherein F¹ is a small molecule fragment moiety comprising an alkyne moiety, a fluorophore moiety, a labeling group, or a combination thereof; and LG is a leaving group moiety. In some embodiments, F¹ comprises an alkyne moiety. In some embodiments, F¹ comprises a fluorophore moiety. In some embodiments, LG comprises a succinimide moiety or a phenyl moiety. In some embodiments, LG comprises the phenyl moiety. In some embodiments, the phenyl moiety comprises one or more substituents selected from the group consisting of halogen, C₁-C₆fluoroalkyl, —CN, —NO₂, —S(═O)R¹, —S(═O)₂R¹, —S(═O)₂OM, —N(R¹)S(═O)₂R¹, —S(═O)₂NR¹R², —C(═O)R¹, —C(═O)OM, —OC(═O)R¹, —C(═O)OR², —OC(═O)OR², —C(═O)NR¹R², —OC(═O)NR¹R², —NR C(═O)NR R², and —NR¹C(═O)R¹; each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl; R² is independently selected from the group consisting of H, D, C₁-C₆alkyl, C₁-C₆fluoroalkyl, C₁-C₆heteroalkyl, and a substituted or unsubstituted aryl; or R¹ and R⁶ are taken together with the intervening atoms joining R⁵ and R⁶ to form a 5- or 6-membered ring; and M is Li, Na, K, or —N(R²)₄. In some embodiments, the probe compound has a structure selected from:

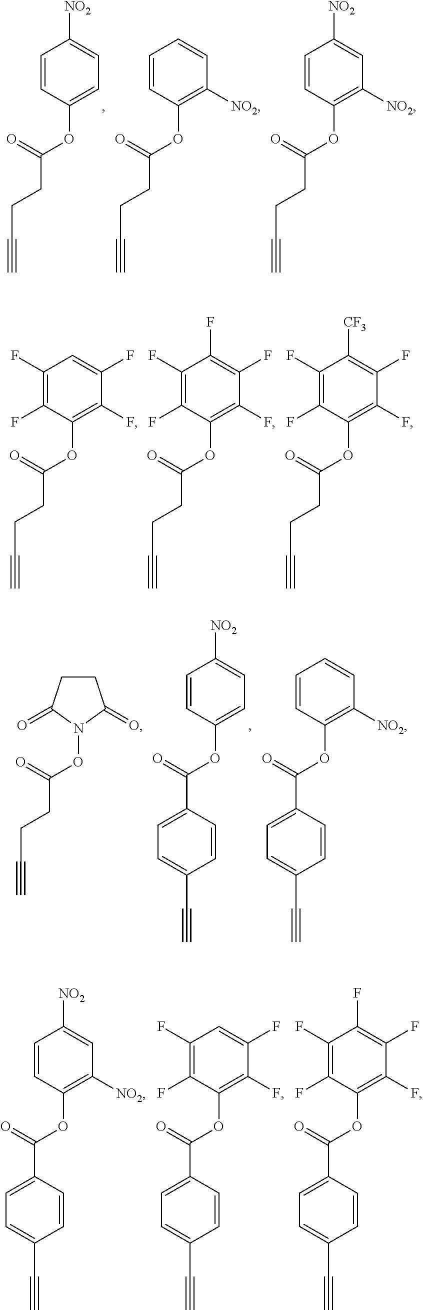

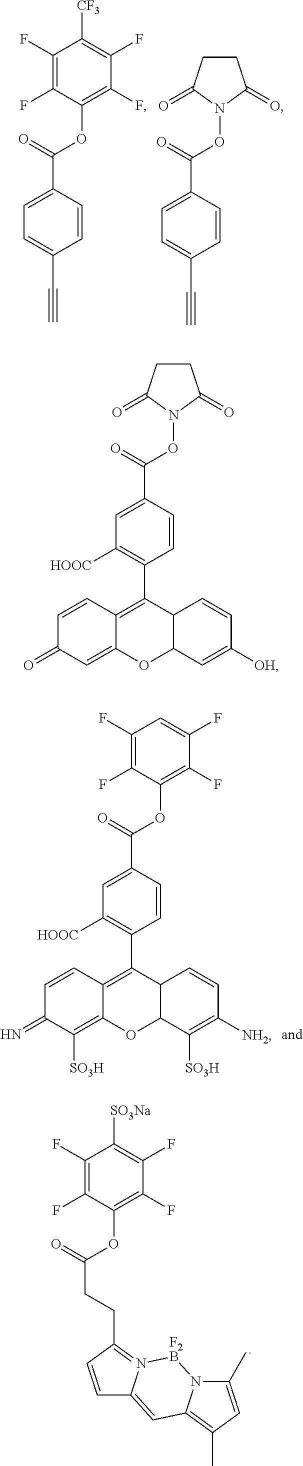

In some embodiments, the analyzing of step (c) further comprises tagging at least one lysine-containing protein-ligand complex of step (b) to generate a tagged lysine-containing protein-ligand complex. In some embodiments, the analyzing of step (c) further comprises isolating the tagged lysine-containing protein-ligand complex. In some embodiments, the tagging comprises attaching a biotin moiety. In some embodiments, the biotin moiety comprises biotin or a biotin derivative. In some embodiments, the biotin derivative comprises desthiobiotin, biotin alkyne or biotin azide. In some embodiments, the biotin moiety comprises desthiobiotin.

Disclosed herein, in certain embodiments, is a method of identifying a protein that interacts with a ligand of interest, comprising: (a) providing a protein sample comprising isolated proteins, living cells, or a cell lysate and separating the protein sample into a first protein sample and a second protein sample; (b) contacting the first protein sample with a ligand for a sufficient time for the ligand to react with a reactive lysine of the first protein sample; (c) contacting the first protein sample and the second protein sample with a probe compound of Formula (I) for a sufficient time for the probe compound to react with the reactive lysines of the first and second protein samples; (d) analyzing the proteins of the first and second protein samples to identify the reactive lysines that bound with the probe compound; (e) comparing the reactivity of the reactive lysine from the first protein sample to the reactivity of the reactive lysine from the second protein sample, wherein a decrease in the reactivity of the reactive lysine of the first protein sample relative to the reactive lysine of the second protein sample indicates interaction of the ligand with the reactive lysine of the first protein sample; and (f) determining the protein comprising the reactive lysine of the first protein sample that interacts with the ligand; wherein the probe compound has a structure represented by Formula (I):

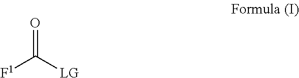

wherein F¹ is a small molecule fragment moiety comprising an alkyne moiety, a fluorophore moiety, a labeling group, or a combination thereof; and LG is a leaving group moiety. In some embodiments, the ligand in step (b) comprises a small molecule compound, a polynucleotide, a polypeptide or its fragments thereof, or a peptidomimetic. In some embodiments, the ligand in step (b) comprises a small molecule compound. In some embodiments, the small molecule compound comprises a ligand-electrophile compound that has a structure represented by Formula (II):

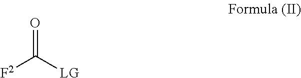

wherein F² is a small molecule fragment moiety; and LG is a leaving group moiety. In some embodiments, F² comprises C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, or a substituted or unsubstituted heteroaryl. In some embodiments, the ligand-electrophile compound has a structure selected from:

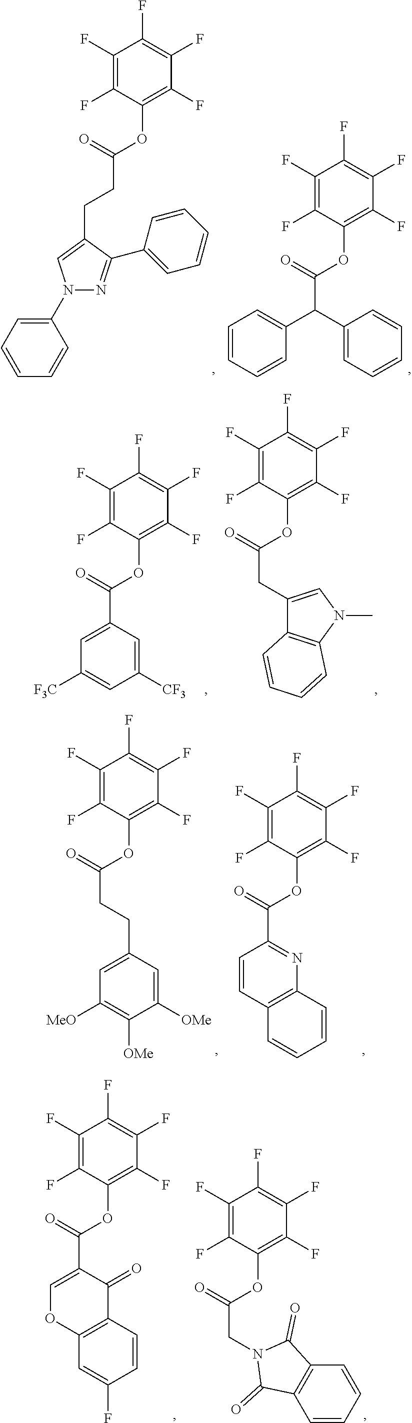

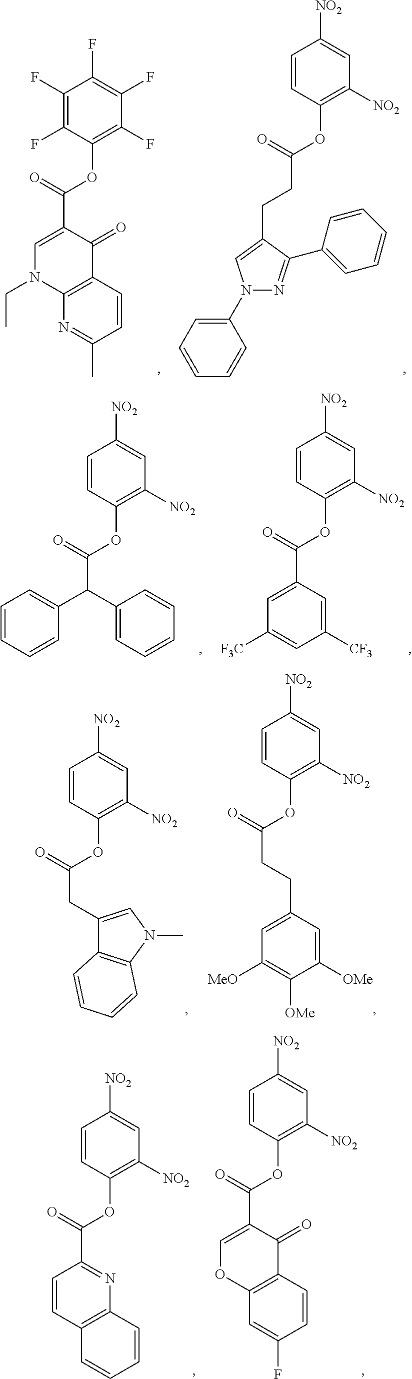

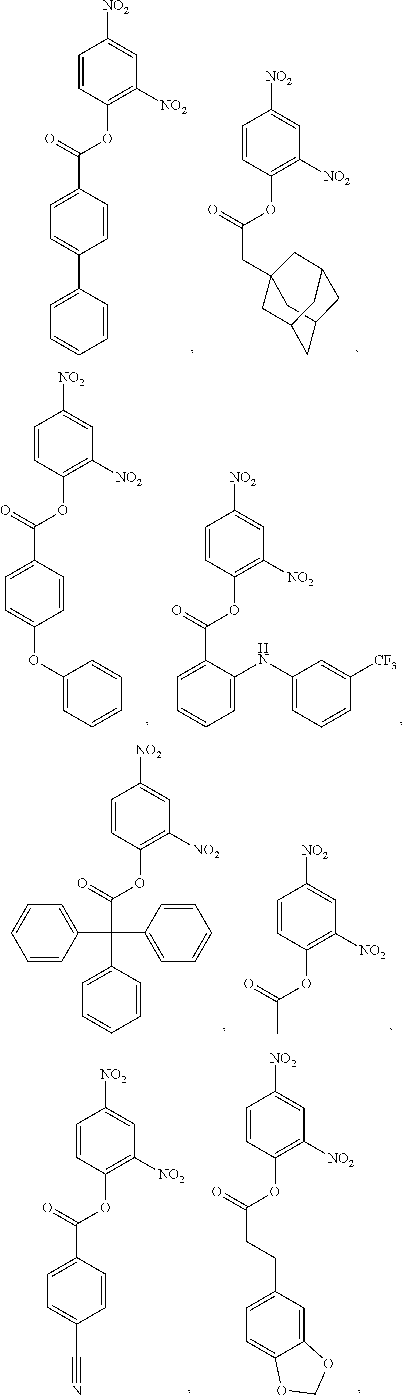

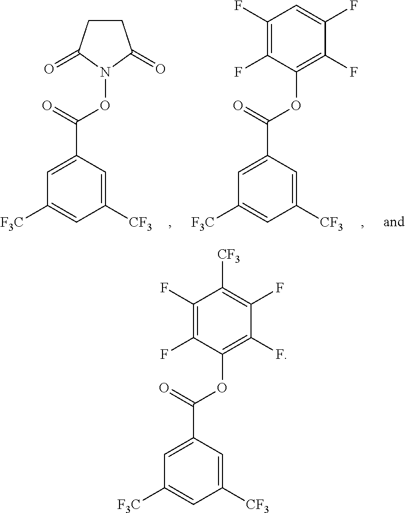

In some embodiments, F² comprises one or more —C(═O)LG moieties. In some embodiments, the ligand-electrophile compound has a structure selected from:

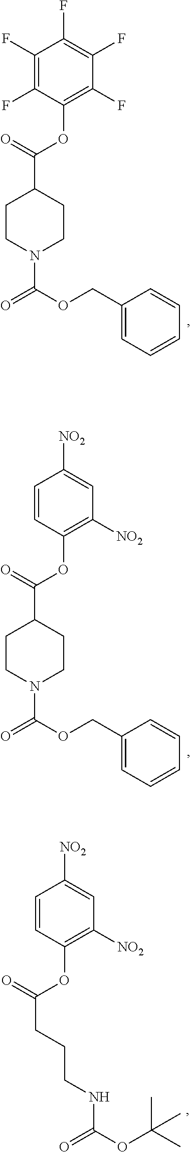 [SMILES: O=C(Oc1c(F)c(F)c(F)c(F)c1F)C1CCN(C(=O)OCc2ccccc2)CC1 ; O=C(Oc1ccc([N+](=O)[O-])cc1[N+](=O)[O-])C1CCN(C(=O)OCc2ccccc2)CC1 ; CC(C)(C)OC(=O)NCCCC(=O)Oc1ccc([N+](=O)[O-])cc1[N+](=O)[O-]]

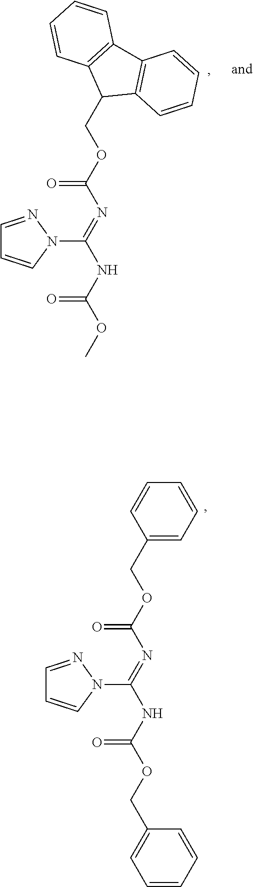 [SMILES: COC(=O)N/C(=N/C(=O)OCC1c2ccccc2-c2ccccc21)n1cccn1 ; O=C(/N=C(/NC(=O)OCc1ccccc1)n1cccn1)OCc1ccccc1]

In some embodiments, the ligand in step (b) comprises a polypeptide or its fragments thereof. In some embodiments the polypeptide is a natural polypeptide. In some embodiments, the polypeptide is an unnatural polypeptide. In some embodiments, the ligand in step (b) comprises a polynucleotide. In some embodiments, the ligand in step (b) comprises a peptidomimetic.

In some embodiments, the analyzing of step (d) further comprises tagging at least one lysine-containing protein-ligand complex of step (c) to generate a tagged lysine-containing protein-ligand complex. In some embodiments, the analyzing of step (d) further comprises isolating the tagged lysine-containing protein-ligand complex. In some embodiments, the tagging comprises attaching a biotin moiety. In some embodiments, the biotin moiety comprises biotin or a biotin derivative. In some embodiments, the biotin derivative comprises desthiobiotin, biotin alkyne or biotin azide. In some embodiments, the biotin moiety comprises desthiobiotin.

Disclosed herein, in certain embodiments, are modified lysine-containing proteins comprising: a small molecule fragment moiety, covalently bonded to a lysine residue of a lysine-containing protein, wherein a covalent bond is formed by reaction with a non-naturally occurring small molecule probe having a structure of Formula (I):

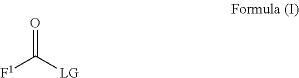

wherein, F¹ is a small molecule fragment moiety comprising an alkyne moiety, a fluorophore moiety, a labeling group, or a combination thereof; and LG is a leaving group moiety. In some embodiments, the lysine residue is attached to the small molecule fragment through an amide bond. In some embodiments, F¹ comprises an alkyne moiety. In some embodiments, F¹ comprises a fluorophore moiety. In some embodiments, LG comprises a succinimide moiety or a phenyl moiety. In some embodiments, LG comprises the phenyl moiety. In some embodiments, the phenyl moiety comprises one or more substituents selected from the group consisting of halogen, C₁-C₆fluoroalkyl, —CN, —NO₂, —S(═O)R¹, —S(═O)₂R¹, —S(═O)₂OM, —N(R¹)S(═O)₂R¹, —S(═O)₂NR¹R², —C(═O)R¹, —C(═O)OM, —OC(═O)R¹, —C(═O)OR², —OC(═O)OR², —C(═O)NR¹R², —OC(═O)NR¹R², —NR¹C(═O)NR¹R², and —NR¹C(═O)R¹; each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl; R² is independently selected from the group consisting of H, D, C₁-C₆alkyl, C₁-C₆fluoroalkyl, C₁-C₆heteroalkyl, and a substituted or unsubstituted aryl; or R¹ and R⁶ are taken together with the intervening atoms joining R⁵ and R⁶ to form a 5- or 6-membered ring; and M is Li, Na, K, or —N(R²)₄. In some embodiments, the small molecule probe has a structure selected from:

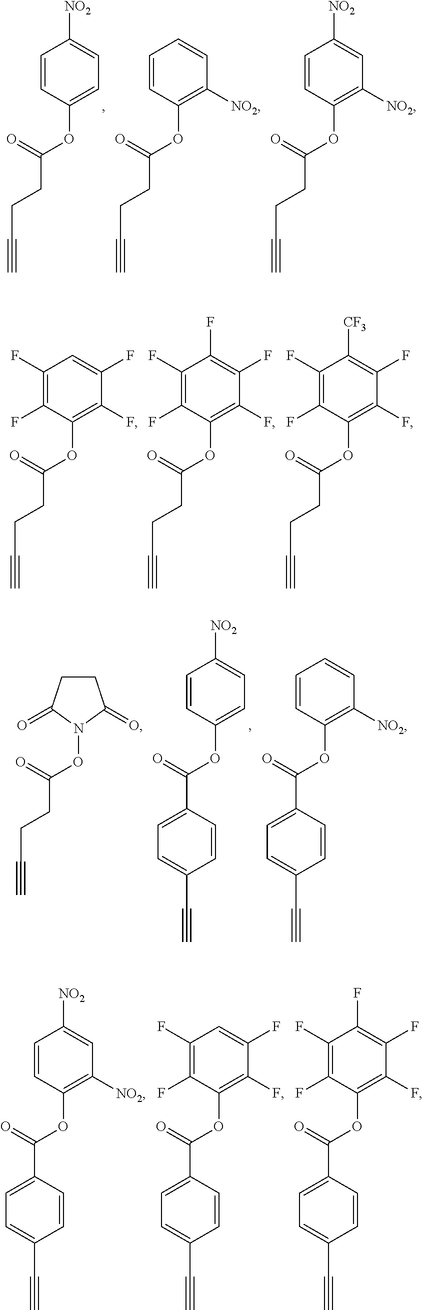

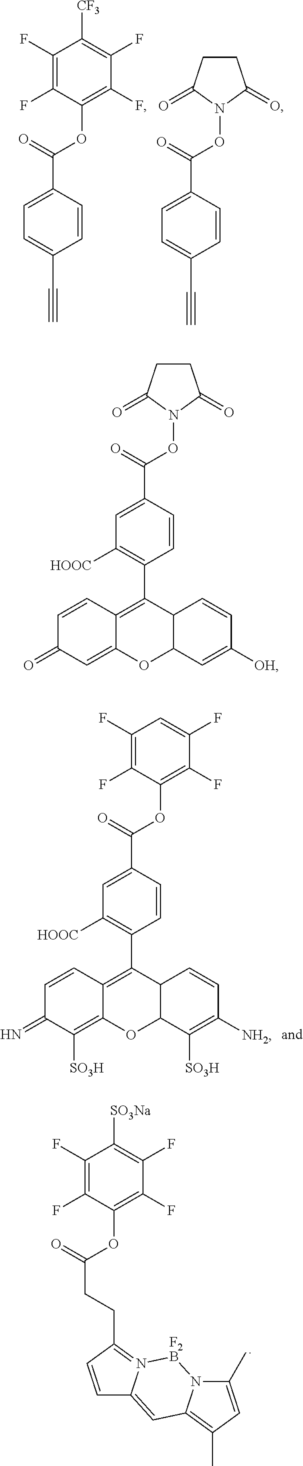

In some embodiments, the labeling group is a biotin moiety. In some embodiments, the biotin moiety comprises biotin or a biotin derivative. In some embodiments, the biotin derivative comprises desthiobiotin, biotin alkyne or biotin azide. In some embodiments, the biotin moiety comprises desthiobiotin. In some embodiments, the lysine-containing protein is a protein selected from Table 1. In some embodiments, the lysine-containing protein is a protein selected from Table 2. In some embodiments, the lysine-containing protein is a protein selected from Table 3.

Disclosed herein, in certain embodiments, are modified lysine-containing proteins comprising: a small molecule fragment moiety, covalently bonded to a lysine residue of a lysine-containing protein, wherein a covalent bond is formed by reaction with a non-naturally occurring ligand-electrophile having a structure of Formula (II):

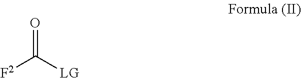

wherein, F² is a small molecule fragment moiety; and LG is a leaving group moiety. In some embodiments, the lysine residue is attached to the small molecule fragment through an amide bond. In some embodiments, F² comprises C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, or a substituted or unsubstituted heteroaryl. In some embodiments, the ligand-electrophile has a structure selected from:

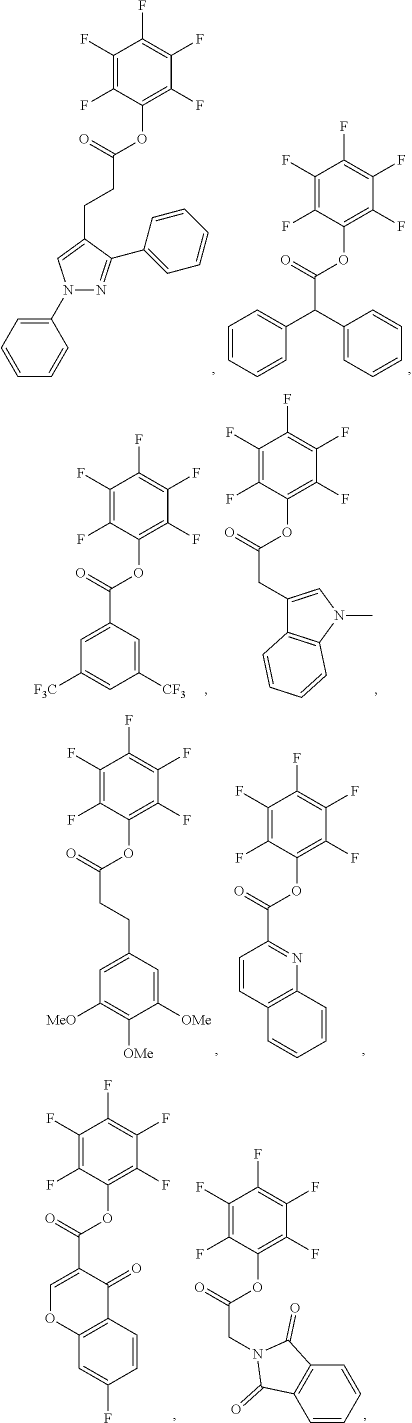

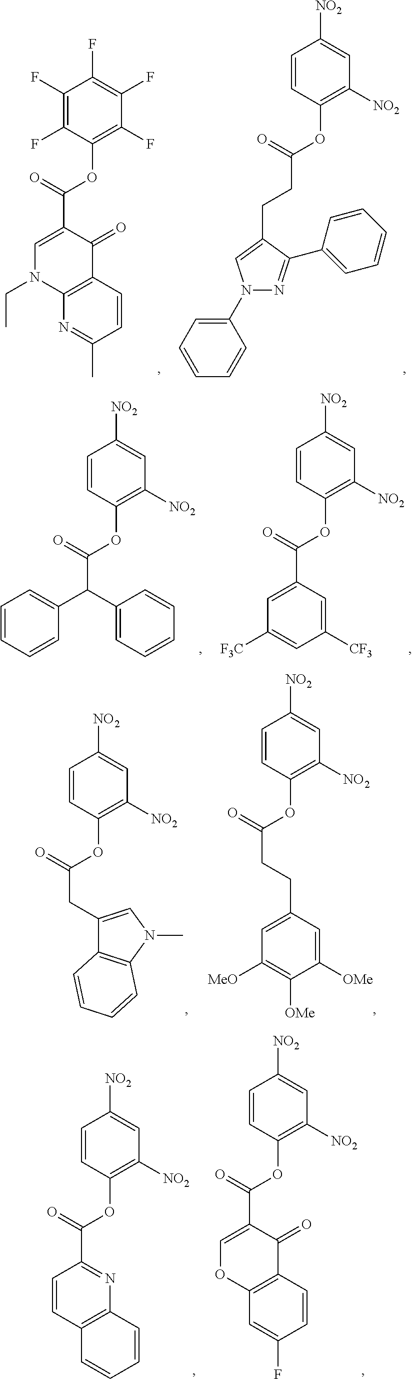

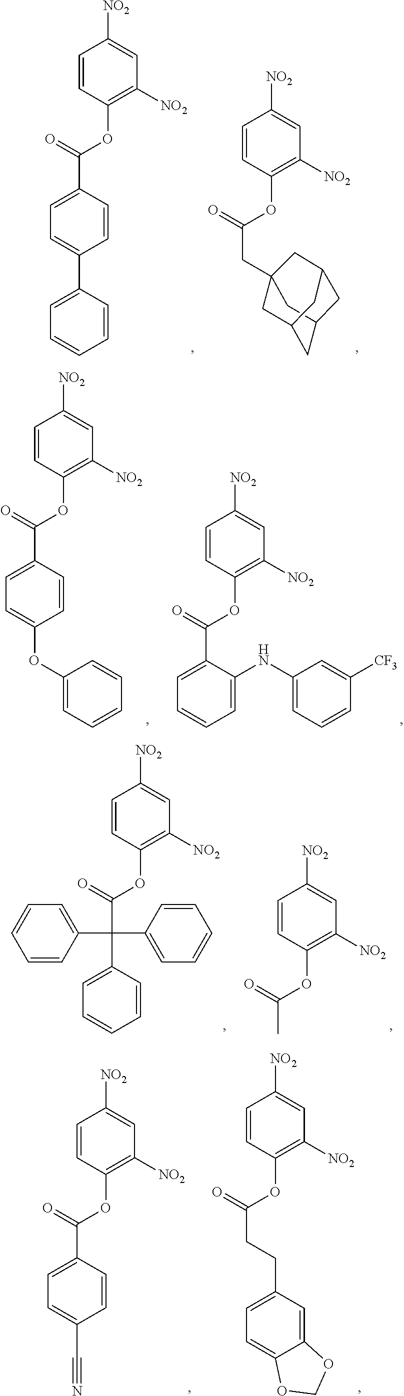

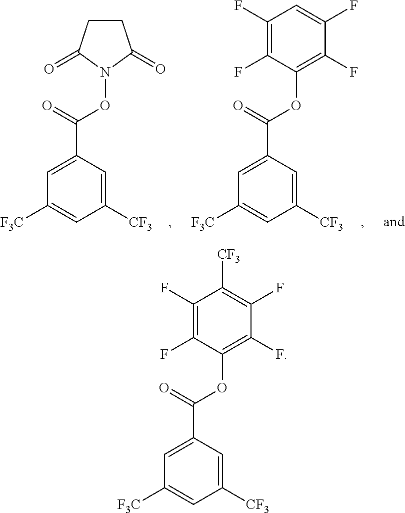

In some embodiments, F² comprises one or more —C(═O)LG moieties. In some embodiments, the ligand-electrophile compound has a structure selected from:

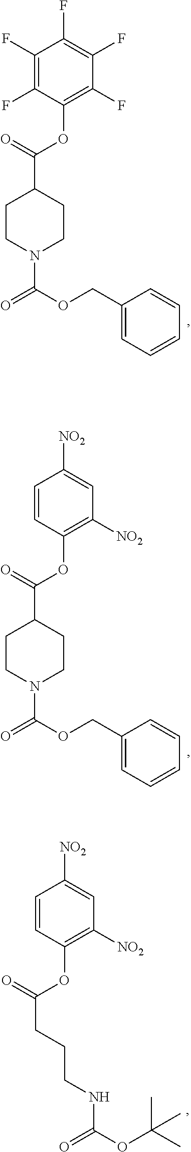 [SMILES: O=C(Oc1c(F)c(F)c(F)c(F)c1F)C1CCN(C(=O)OCc2ccccc2)CC1 ; O=C(Oc1ccc([N+](=O)[O-])cc1[N+](=O)[O-])C1CCN(C(=O)OCc2ccccc2)CC1 ; CC(C)(C)OC(=O)NCCCC(=O)Oc1ccc([N+](=O)[O-])cc1[N+](=O)[O-]]

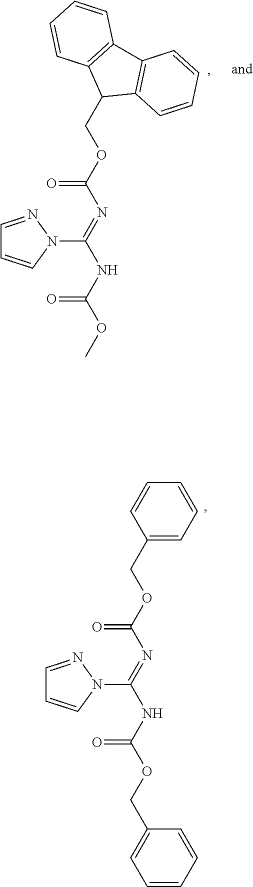 [SMILES: COC(=O)N/C(=N/C(=O)OCC1c2ccccc2-c2ccccc21)n1cccn1 ; O=C(/N=C(/NC(=O)OCc1ccccc1)n1cccn1)OCc1ccccc1]

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1E illustrate proteome-wide quantification of lysine reactivity. FIG. 1A illustrates general protocol for lysine reactivity profiling by isoTOP-ABPP. FIG. 1B illustrates probe 1 preferentially labels lysine residues in human cell proteomes. FIG. 1C illustrates R values for probe 1-labeled peptides from human cancer cell proteomes. FIG. 1D illustrates number of hyper-reactive and quantified lysines per protein shown for proteins found to contain at least one hyper-reactive lysine. FIG. 1E illustrates hyper-reactive lysines are site-selectively labeled by activated ester probes.

FIG. 2A-FIG. 2D illustrate global and specific assessments of the functionality of lysine reactivity. FIG. 2A illustrates distribution of functional classes of proteins that contain hyper-reactive lysines compared to other quantified proteins lacking hyper-reactive lysines. FIG. 2B illustrates hyper-reactive lysines are enriched proximal to (within 10 Å of) annotated functional sites for proteins that have x-ray or NMR structures in the Protein Data. FIG. 2C illustrates hyper-reactive lysines are less likely to be ubiquitylated than lysines of lower reactivity. FIG. 2D illustrates mutation of hyper-reactive lysines blocks the catalytic activity of NUDT2 and G6PD and reduces the activity of PFKP.

FIG. 3A-FIG. 3H illustrate proteome-wide screening of lysine-reactive fragment electrophiles. FIG. 3A illustrates general protocol for competitive isoTOP-ABPP. FIG. 3B illustrates non-limiting examples of general structures of a lysine-reactive, electrophilic fragment library. FIG. 3C illustrates fraction of total quantified lysines and proteins that were liganded by fragment electrophiles in competitive isoTOP-ABPP experiments (left panel). of the liganded proteins, the fraction that is found in Drugbank (middle panel). functional classes of liganded Drugbank and non-Drugbank proteins (right panel). FIG. 3D illustrates number of liganded and quantified lysines per protein measured by isoTOP-ABPP. FIG. 3E illustrates R values for ten lysines in PFKP quantified by isoTOP-ABPP, identifying K688 as the only liganded lysine in this protein. FIG. 3F illustrates comparison of the ligandability of lysine residues as a function of their reactivity with probe 1. FIG. 3G illustrates lysine reactivity distribution for both liganded and unliganded lysine residues labeled by probe 1. FIG. 3H illustrates overlap of proteins harboring liganded lysines and liganded cysteines.

FIG. 4A-FIG. 4B illustrate analysis of fragment-lysine interactions. FIG. 4A illustrates heat-map showing R values for representative lysines and fragments organized by relative proteomic reactivity of the fragments (high to low, left to right) and number of fragment hits for individual lysines (high to low, top to bottom). FIG. 4B illustrates fragment SAR determined by competitive isoTOP-ABPP is recapitulated by gel-based ABPP of recombinant proteins. left panel, heat-map depicts R values for the indicated fragment-lysine interactions determined by competitive isoTOP-ABPP. right panel, HEK 293T cells recombinantly expressing representative liganded proteins.

FIG. 5A-FIG. 5B illustrate confirmation of site-specific fragment-lysine reactions by MS-based proteomics. FIG. 5A illustrates schematic workflow for direct measurement of lysine-fragment reactions on proteins by quantitative proteomics. FIG. 5B illustrates R values for all detected, unmodified lysine-containing tryptic peptides for representative liganded proteins after treatment with the indicated compounds.

FIG. 6A-FIG. 6I illustrate fragment-lysine reactions inhibit the function of diverse proteins. FIG. 6A-FIG. 6C illustrate fragments targeting active site (PNPO and NUDT2) and allosteric (PFKP) lysines in metabolic enzymes block enzymatic activity in a concentration-dependent manner with apparent IC₅₀ values comparable to those measured by gel-based ABPP with lysine-reactive probes (probe labeling). FIG. 6D illustrates the liganded lysine K155 in SIN3A (red) is located at the protein-protein interaction site of the PAH1 domain (green). FIG. 6E illustrates fragment 21 (50 μM) fully competes probe 1 labeling of K155 of SIN3A as determined by isoTOP-ABPP of human cancer cell proteomes. FIG. 6F illustrates gel-based ABPP confirms that 21 blocks probe 17 labeling of SIN3A at K155 in a concentration-dependent manner. FIG. 6G illustrates heat-map showing the enrichment of SIN3A-interacting proteins in co-immunoprecipitation-MS-based proteomic experiments. FIG. 6H and FIG. 6I illustrate flag-SIN3A or the indicated Flag-SIN3A mutants (a.a. 1-400), or Flag-GFP, were co-expressed in HEK 293T cells with Myc-TGIF1 or Myc-TGIF2. Representative western blots are shown in FIG. 6H, and quantification for four biological replicates is provided in FIG. 6I.

FIG. 7A-FIG. 7C illustrate evaluation of lysine-reactive probes for isoTOP-ABPP. FIG. 7A illustrates structures of various alkyne-(2-15) and fluorophore-(16-18) modified, amine-reactive probes (see FIG. 1A for the structure of STP-alkyne probe 1). FIG. 7B illustrates qualitative assessment of respective proteomic reactivities of probes by SDS-PAGE and in-gel fluorescence scanning of MDA-MB-231 lysates. FIG. 7C illustrates most peptides detected as labeled by probe 1 on residues other than lysine contain missed tryptic cleavage events at unmodified lysine residues.

FIG. 8A-FIG. 8H illustrate proteome-wide quantification of lysine reactivity. FIG. 8A illustrates overlap of probe 1-labeled peptides detected in isoTOP-ABPP experiments performed with proteomes from the three indicated human cancer cell lines. FIG. 8B illustrates probe 1 also exhibits high selectivity for reacting with lysine in isoTOP-ABPP experiments comparing MDA-MB-231 cell lysates. FIG. 8C-FIG. 8F illustrate consistency of lysine reactivity ratios (R values) for isoTOP-ABPP experiments comparing 0.1 and 1.0 mM of probe 1 with (c) biological replicates of the same proteome (MDA-MB-231 lysates), or (FIG. 8D-FIG. 8F) proteomes from three different human cancer cell lines (MDA-MB-231, Ramos and Jurkat cells). FIG. 8G illustrates R values for hyper-reactive (red) and medium/low-reactivity (black) lysines found within the same protein. FIG. 8H illustrates hyper-reactive lysines might be site-selectively labeled by activated ester probes.

FIG. 9A-FIG. 9G illustrate global and specific assessments of probe 1-reactive lysines. FIG. 9A illustrates box and whiskers plot showing the distribution of lysine conservation across M. musculus, X. laevis, D. malanogaster, C. elegans and D. rerio for probe 1-labeled lysines from different reactivity groups. FIG. 9B illustrates frequency plots showing no apparent conserved motifs for lysines from different reactivity groups. FIG. 9C illustrates hyper-reactive lysines are enriched near pockets. FIG. 9D illustrates hyper-reactive lysines are less likely to be acetylated than lysines of lower reactivity. FIG. 9E-FIG. 9G illustrate structures of proteins with hyper-reactive lysines. Hyper-reactive lysines (K89 for NUDT2, K171 for G6PD and K688 for PFKP) are shown in red and molecules bound in the active site of the protein in orange (ATP for NUDT2, glucose-6-phosphate for G6PD and AMPPCP for PFKP).

FIG. 10A-FIG. 10D illustrate proteome-wide screening of lysine-reactive fragment electrophiles. FIG. 10A-FIG. 10B illustrate structures of compounds in the lysine-reactive fragment electrophile library, including non-electrophilic, amide-containing control compound 51 (b). FIG. 10C illustrates frequency of quantification of all lysines for the competitive isoTOP-ABPP experiments performed with fragment electrophiles. FIG. 10D illustrates R values for six lysine residues in hexokinase-1 (HK1) quantified by isoTOP-ABPP, identifying K510 as the only liganded lysine in HK1. Each point represents a distinct fragment-lysine interaction quantified by isoTOP-ABPP.

FIG. 11A-FIG. 1G illustrate lysine-reactive fragment electrophiles exhibit distinct proteome-wide reactivity profiles. FIG. 11A illustrates that most liganded lysines are targeted by a limited subset (<10%) of the fragment electrophiles. Histogram depicting the number of liganded lysines targeted by different percentages of fragments. Percentage is the fraction of ligands among the fragments that this lysine was quantified for. FIG. 11B illustrates the rank order of proteomic reactivity values for fragment electrophiles calculated as the percentage of all quantified lysines with R values ≥4 for each fragment. FIG. 11C illustrates the rank order of reactivity values of fragment electrophiles calculated as the percentage of all liganded lysines with R values ≥4 for each fragment. FIG. 11D illustrates an average proteomic reactivity values for eight pentafluorophenyl and eight dinitrophenyl esters that share common fragment-based binding elements. FIG. 11E illustrates Western blot analysis confirming equivalent protein expression for gel-based ABPP experiments depicted in FIG. 10B. FIG. 11F illustrates heat-map showing proteins that interact preferentially with dinitrophenyl and pentafluorophenyl esters, respectively. FIG. 1G illustrates probe 1-labeling of K89 in NUDT2 is quantitatively blocked by guanidinylating fragment electrophile 49, but not by the three tested activated ester fragment electrophiles.

FIG. 12A-FIG. 12J illustrates site-specific fragment-lysine reactions and their functional effects on proteins. FIG. 12A illustrates the structure of PNPO (PDB ID: 1NRG). Hyper-reactive lysine K100 is shown in red and FMN and pyridoxal-5′-phosphate bound in the active site are shown in orange. FIG. 12B-FIG. 12G, illustrate competitive isoTOP-ABPP analysis. FIG. 12 B, FIG. 12D, and FIG. 12F of MDA-MB-231 cell lysate treated with the indicated fragment electrophiles followed by probe 1 in PNPO (FIG. 12B), PFKP (FIG. 12D), and NUDT2 (FIG. 12F); FIG. 12C, FIG. 12E, and FIG. 12G illustrate lysates from HEK 293T cells recombinantly expressing PNPO (FIG. 12C), NUDT2 (FIG. 12E), and PFKP (FIG. 12G) or the indicated lysine-to-arginine mutants. FIG. 12H illustrates fragment 20 blocks the catalytic activity of PFKP in a concentration-dependent manner to produce a maximal inhibitory effect of about 80%. FIG. 12I illustrates IC₅₀ curve for blockade of probe 17-labeling of SIN3A by fragment electrophile 21. FIG. 12J illustrates flag-SIN3A or the indicated Flag-SIN3A mutants (a.a. 1-400), or Flag-GFP, were co-expressed in HEK 293T with Myc-TGIF2.

### DETAILED DESCRIPTION OF THE DISCLOSURE

Lysine containing proteins encompass a large repertoire of proteins that participate in numerous cellular functions and are found at many functional sites, including enzyme active sites and at interfaces mediating protein-protein interactions. Lysines also serve as sites for post-translational regulation of protein structure and function through, for instance, acetylation, methylation, and ubiquitylation. In some instances, about 9000 lysines are quantified in human cell proteomes and about several hundred residues with heightened reactivity are identified that are enriched at protein functional sites.

Small molecules serve as versatile probes for perturbing the functions of proteins in biological systems. In some instances, a plurality of human proteins lack selective chemical ligands. In some cases, several classes of proteins are further considered as undruggable. Covalent ligands offer a strategy to expand the landscape of proteins amenable to targeting by small molecules. In some instances, covalent ligands combine features of recognition and reactivity, thereby enabling targeting sites on proteins that are difficult to address by reversible binding interactions alone.

Described herein are small molecule probes that interact with a reactive lysine residue of a lysine-containing protein and methods of identifying a protein that contains such a reactive lysine residue (e.g., a druggable lysine residue). In some instances, also described herein are methods of profiling a ligand that interacts with one or more lysine-containing proteins comprising reactive lysines.

Described herein are modified lysine-containing proteins that are formed by reaction of a lysine-cotaining protein with one or more probes, ligands, ligand-electrophiles, or other moiety comprising a chemical group capable of reacting with a lysine residue. Further described herein are modified-lysine-containing proteins covalently attached to a small molecule fragment moiety via an amide linkage. Further described herein are kits for generating modified lysine-containing proteins.

Small Molecule Probe Compounds

In some embodiments, the small molecule probe compound described herein comprises a reactive moiety which interacts with the amino group of a lysine residue of a lysine containing protein. In some instances, small molecule probes react with lysine residues to form covalent bonds. Often, small molecule probes are non-naturally occurring, or form non-naturally occurring products after reaction with the amino group of a lysine residue of a lysine containing protein. In some instances, the amino group of the lysine-containing protein is connected to a small molecule fragment moiety via an amide bond after reaction with a small molecule probe.

In some embodiments, a small molecule probe compound described herein is a small molecule compound that has a structure represented by Formula (I):

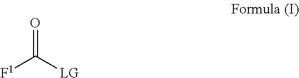

wherein,
F¹ is a small molecule fragment moiety comprising an alkyne moiety, a fluorophore moiety, a labeling group, or a combination thereof; and
LG is a leaving group moiety.

In some embodiments, the fluorophore comprises rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ Rho 101, ATTO™ 590, ATTO™ 633, ATTO™ 647N, TYE™ 563, TYE™ 665, or TYE™ 705.

In some embodiments, the labeling group is biotin moiety, streptavidin moiety, bead, resin, a solid support, or a combination thereof.

In some embodiments, F¹ comprises a fluorophore moiety. In some cases, F¹ is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

Leaving groups (leaving group moiety, LG) variously comprise any number of chemical groups capable of stabilizing a negative charge. LG in some embodiments comprise alkoxy, aryloxy, arylthiols, thiols, oxyamine, or other group. LG is in some cases charged, such as those comprising ammonium, pyridinium, sulfate, phosphate, or other cationic or anionic groups. In some embodiments, LG comprises electron-withdrawing groups such as NO₂, F, CF₃, SO₃ or other electron-withdrawing group. In some embodiments, LG comprises a succinimide moiety or a phenyl moiety. In some embodiments, LG comprises a succinimide moiety. In some embodiments, LG comprises a phenyl moiety.

In some embodiments, the phenyl moiety comprises one or more substituents selected from the group consisting of halogen, C₁-C₆fluoroalkyl, —CN, —NO₂, —S(═O)R¹, —S(═O)₂R¹, —S(═O)₂OM, —N(R¹)S(═O)₂R¹, —S(═O)₂NR¹R², —C(═O)R¹, —C(═O)OM, —OC(═O)R¹, —C(═O)OR², OC(═O)OR², —C(═O)NR¹R², —OC(═O)NR¹R², —NR¹C(═O)NR¹R², and —NR¹C(═O)R¹;
each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl;
R² is independently selected from the group consisting of H, D, C₁-C₆alkyl, C₁-C₆fluoroalkyl, C₁-C₆heteroalkyl, and a substituted or unsubstituted aryl;
or R¹ and R⁶ are taken together with the intervening atoms joining R⁵ and R⁶ to form a 5- or 6-membered ring; and
M is Li, Na, K, or —N(R²)₄.

In some instances, a small molecule probe compound of Formula (I) has a structure selected from:

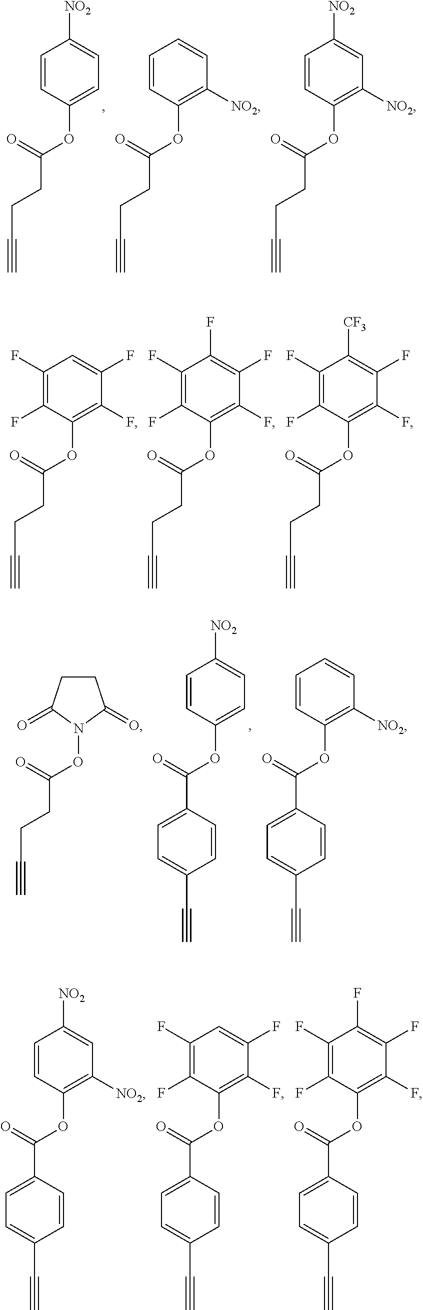

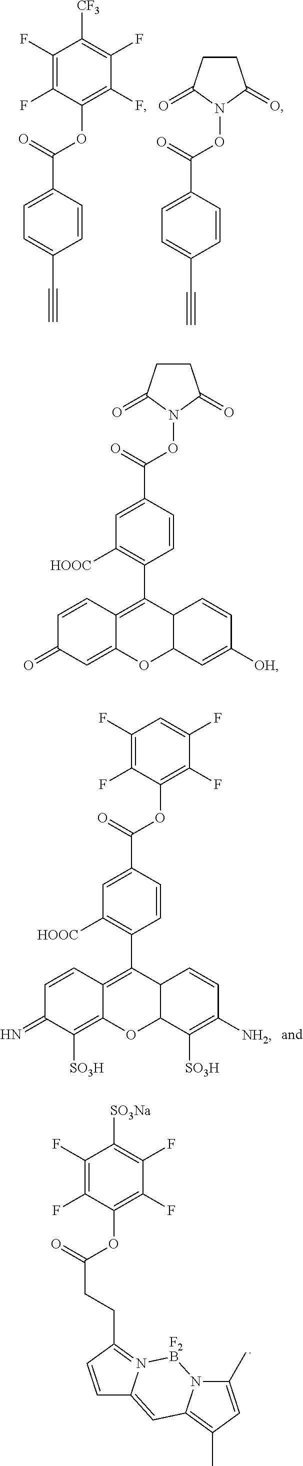

Ligand

In some embodiments, a ligand competes with a probe compound described herein for binding with a reactive lysine residue. In some instances, a ligand comprises a small molecule compound, a polynucleotide, a polypeptide or its fragments thereof, or a peptidomimetic. In some embodiments, the ligand comprises a small molecule compound. In some instances, a small molecule compound comprises a fragment moiety that facilitates interaction of the compound with a reactive lysine residue. In some cases, a small molecule compound comprises a small molecule fragment that facilitates hydrophobic interaction, hydrogen bonding, or a combination thereof. Often, ligands are non-naturally occurring, or form non-naturally occurring products after reaction with the amino group of a lysine residue of a lysine containing protein. In some instances, a ligand comprises a small-molecule compound. In some embodiments, a small molecule compound comprises a ligand-electrophile. Such ligand-electrophiles often reaction with the amino group of a lysine residue of a lysine-containing protein.

In some embodiments, a ligand comprises a polynucleotide. In some instances, the polynucleotide comprises an endogenous substrate that interacts with a lysine-containing protein. In some instances, the polynucleotide comprises modified and/or synthetic substrate. In some cases, the polynucleotide comprises natural nucleotides. In other cases, the polynucleotide comprises artificial nucleotides.

In some instances, a polynucleotide comprises from about 8 to about 50 bases in length. In some cases, a polynucleotide comprises from about 12 to about 45, from about 15 to about 40, from about 20 to about 40, or from about 25 to about 300 bases in length. In some cases, a polynucleotide comprises 8, 9, 10, 11, 12, 13, 14, 15, 16, 17, 18, 19, 20, 21, 22, 23, 24, 25, 30, 35, 40, 45, or 50 bases in length.

In some embodiments, a ligand comprises a polypeptide or its fragments thereof. In some instances, the polypeptide comprises a wild-type functional protein, protein variants, or mutants that are substrates for a lysine-containing protein of interest. In some instances, fragments of the polypeptide comprise truncated functional proteins that interact with the lysine-containing protein of interest.

In some instances, a functional fragment of a polypeptide comprises from about 10 to about 80 amino acid residues in length. In some instances, the functional fragment comprises from about 15 to about 70, from about 20 to about 60, from about 30 to about 50, or from about 40 to about 80 amino acid residues in length. In some cases, the functional fragment comprises about 10, 15, 20, 25, 30, 35, 40, 45, 50, 55, 60, 70, 80, or more amino acid residues in length.

In some cases, a polypeptide or its fragments thereof comprise natural amino acids, unnatural amino acids, or a combination thereof. In some cases, the polypeptide or its fragments thereof comprise L-amino acids, D-amino acids, or a combination thereof.

In some instances, a ligand comprises a peptidomimetic. Peptidomimetic is a small protein-like chain that mimics a peptide. Exemplary peptidomimetics include, but are not limited to, peptoids, β-peptides, or foldamers. Peptoids, also known as poly-N-substituted glycines, are a class of peptidomimetics in which the side chains are appended to the nitrogen atom of the peptide backbone instead of the α-carbon. β-peptides are β-amino acids in which the amino groups are bonded to the β-carbon rather than the α-carbon. A foldamer is a discrete chain molecule or oligomer that folds into an ordered conformation such as helices and β-sheets.

As referred to above, exemplary unnatural amino acid residues comprise, for example, amino acid analogs such as β-amino acid analogs; racemic analogs; or analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary β-amino acid analogs include, but are not limited to, cyclic β-amino acid analogs, β-alanine, (R)-β-phenylalanine, (R)-1,2,3,4-tetrahydro-isoquinoline-3-acetic acid, (R)-3-amino-4-(1-naphthyl)-butyric acid, (R)-3-amino-4-(2,4-dichlorophenyl)butyric acid, (R)-3-amino-4-(2-chlorophenyl)-butyric acid, (R)-3-amino-4-(2-cyanophenyl)-butyric acid, (R)-3-amino-4-(2-fluorophenyl)-butyric acid, (R)-3-amino-4-(2-furyl)-butyric acid, (R)-3-amino-4-(2-methylphenyl)-butyric acid, (R)-3-amino-4-(2-naphthyl)-butyric acid, (R)-3-amino-4-(2-thienyl)-butyric acid, (R)-3-amino-4-(2-trifluoromethylphenyl)-butyric acid, (R)-3-amino-4-(3,4-dichlorophenyl)butyric acid, (R)-3-amino-4-(3,4-difluorophenyl)butyric acid, (R)-3-amino-4-(3-benzothienyl)-butyric acid, (R)-3-amino-4-(3-chlorophenyl)-butyric acid, (R)-3-amino-4-(3-cyanophenyl)-butyric acid, (R)-3-amino-4-(3-fluorophenyl)-butyric acid, (R)-3-amino-4-(3-methylphenyl)-butyric acid, (R)-3-amino-4-(3-pyridyl)-butyric acid, (R)-3-amino-4-(3-thienyl)-butyric acid, (R)-3-amino-4-(3-trifluoromethylphenyl)-butyric acid, (R)-3-amino-4-(4-bromophenyl)-butyric acid, (R)-3-amino-4-(4-chlorophenyl)-butyric acid, (R)-3-amino-4-(4-cyanophenyl)-butyric acid, (R)-3-amino-4-(4-fluorophenyl)-butyric acid, (R)-3-amino-4-(4-iodophenyl)-butyric acid, (R)-3-amino-4-(4-methylphenyl)-butyric acid, (R)-3-amino-4-(4-nitrophenyl)-butyric acid, (R)-3-amino-4-(4-pyridyl)-butyric acid, (R)-3-amino-4-(4-trifluoromethylphenyl)-butyric acid, (R)-3-amino-4-pentafluoro-phenylbutyric acid, (R)-3-amino-5-hexenoic acid, (R)-3-amino-5-hexynoic acid, (R)-3-amino-5-phenylpentanoic acid, (R)-3-amino-6-phenyl-5-hexenoic acid, (S)-1,2,3,4-tetrahydro-isoquinoline-3-acetic acid, (S)-3-amino-4-(1-naphthyl)-butyric acid, (S)-3-amino-4-(2,4-dichlorophenyl)butyric acid, (S)-3-amino-4-(2-chlorophenyl)-butyric acid, (S)-3-amino-4-(2-cyanophenyl)-butyric acid, (S)-3-amino-4-(2-fluorophenyl)-butyric acid, (S)-3-amino-4-(2-furyl)-butyric acid, (S)-3-amino-4-(2-methylphenyl)-butyric acid, (S)-3-amino-4-(2-naphthyl)-butyric acid, (S)-3-amino-4-(2-thienyl)-butyric acid, (S)-3-amino-4-(2-trifluoromethylphenyl)-butyric acid, (S)-3-amino-4-(3,4-dichlorophenyl)butyric acid, (S)-3-amino-4-(3,4-difluorophenyl)butyric acid, (S)-3-amino-4-(3-benzothienyl)-butyric acid, (S)-3-amino-4-(3-chlorophenyl)-butyric acid, (S)-3-amino-4-(3-cyanophenyl)-butyric acid, (S)-3-amino-4-(3-fluorophenyl)-butyric acid, (S)-3-amino-4-(3-methylphenyl)-butyric acid, (S)-3-amino-4-(3-pyridyl)-butyric acid, (S)-3-amino-4-(3-thienyl)-butyric acid, (S)-3-amino-4-(3-trifluoromethylphenyl)-butyric acid, (S)-3-amino-4-(4-bromophenyl)-butyric acid, (S)-3-amino-4-(4-chlorophenyl) butyric acid, (S)-3-amino-4-(4-cyanophenyl)-butyric acid, (S)-3-amino-4-(4-fluorophenyl) butyric acid, (S)-3-amino-4-(4-iodophenyl)-butyric acid, (S)-3-amino-4-(4-methylphenyl)-butyric acid, (S)-3-amino-4-(4-nitrophenyl)-butyric acid, (S)-3-amino-4-(4-pyridyl)-butyric acid, (S)-3-amino-4-(4-trifluoromethylphenyl)-butyric acid, (S)-3-amino-4-pentafluoro-phenylbutyric acid, (S)-3-amino-5-hexenoic acid, (S)-3-amino-5-hexynoic acid, (S)-3-amino-5-phenylpentanoic acid, (S)-3-amino-6-phenyl-5-hexenoic acid, 1,2,5,6-tetrahydropyridine-3-carboxylic acid, 1,2,5,6-tetrahydropyridine-4-carboxylic acid, 3-amino-3-(2-chlorophenyl)-propionic acid, 3-amino-3-(2-thienyl)-propionic acid, 3-amino-3-(3-bromophenyl)-propionic acid, 3-amino-3-(4-chlorophenyl)-propionic acid, 3-amino-3-(4-methoxyphenyl)-propionic acid, 3-amino-4,4,4-trifluoro-butyric acid, 3-aminoadipic acid, D-β-phenylalanine, β-leucine, L-β-homoalanine, L-β-homoaspartic acid γ-benzyl ester, L-β-homoglutamic acid δ-benzyl ester, L-β-homoisoleucine, L-β-homoleucine, L-β-homomethionine, L-β-homophenylalanine, L-β-homoproline, L-β-homotryptophan, L-β-homovaline, L-Nω-benzyloxycarbonyl-β-homolysine, Nω-L-β-homoarginine, O-benzyl-L-β-homohydroxyproline, O-benzyl-L-β-homoserine, O-benzyl-L-β-homothreonine, O-benzyl-L-β-homotyrosine, γ-trityl-L-β-homoasparagine, (R)-β-phenylalanine, L-β-homoaspartic acid γ-t-butyl ester, L-β-homoglutamic acid δ-t-butyl ester, L-Nω-β-homolysine, Nδ-trityl-L-β-homoglutamine, Nω-2,2,4,6,7-pentamethyl-dihydrobenzofuran-5-sulfonyl-L-β-homoarginine, O-t-butyl-L-β-homohydroxy-proline, O-t-butyl-L-β-homoserine, O-t-butyl-L-β-homothreonine, O-t-butyl-L-β-homotyrosine, 2-aminocyclopentane carboxylic acid, and 2-aminocyclohexane carboxylic acid.

In some instances, unnatural amino acid residues comprise a racemic mixture of amino acid analogs. For example, in some instances, the D isomer of the amino acid analog is used. In some cases, the L isomer of the amino acid analog is used. In some instances, the amino acid analog comprises chiral centers that are in the R or S configuration. Sometimes, the amino group(s) of a 3-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a 3-amino acid analog is protected, e.g., as its ester derivative. In some cases, the salt of the amino acid analog is used.

In some cases, unnatural amino acid residues comprise analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary amino acid analogs of alanine, valine, glycine, and leucine include, but are not limited to, α-methoxyglycine, α-allyl-L-alanine, α-aminoisobutyric acid, α-methyl-leucine, β-(1-naphthyl)-D-alanine, β-(1-naphthyl)-L-alanine, β-(2-naphthyl)-D-alanine, β-(2-naphthyl)-L-alanine, β-(2-pyridyl)-D-alanine, β-(2-pyridyl)-L-alanine, β-(2-thienyl)-D-alanine, β-(2-thienyl)-L-alanine, β-(3-benzothienyl)-D-alanine, β-(3-benzothienyl)-L-alanine, β-(3-pyridyl)-D-alanine, β-(3-pyridyl)-L-alanine, β-(4-pyridyl)-D-alanine, β-(4-pyridyl)-L-alanine, β-chloro-L-alanine, β-cyano-L-alanine, β-cyclohexyl-D-alanine, β-cyclohexyl-L-alanine, β-cyclopenten-1-yl-alanine, β-cyclopentyl-alanine, β-cyclopropyl-L-Ala-OH.dicyclohexylammonium salt, β-t-butyl-D-alanine, β-t-butyl-L-alanine, γ-aminobutyric acid, L-α,β-diaminopropionic acid, 2,4-dinitro-phenylglycine, 2,5-dihydro-D-phenylglycine, 2-amino-4,4,4-trifluorobutyric acid, 2-fluoro-phenylglycine, 3-amino-4,4,4-trifluoro-butyric acid, 3-fluoro-valine, 4,4,4-trifluoro-valine, 4,5-dehydro-L-leu-OH.dicyclohexylammonium salt, 4-fluoro-D-phenylglycine, 4-fluoro-L-phenylglycine, 4-hydroxy-D-phenylglycine, 5,5,5-trifluoro-leucine, 6-aminohexanoic acid, cyclopentyl-D-Gly-OH.dicyclohexylammonium salt, cyclopentyl-Gly-OH.dicyclohexylammonium salt, D-α,β-diaminopropionic acid, D-α-aminobutyric acid, D-α-t-butylglycine, D-(2-thienyl)glycine, D-(3-thienyl)glycine, D-2-aminocaproic acid, D-2-indanylglycine, D-allylglycine-dicyclohexylammonium salt, D-cyclohexylglycine, D-norvaline, D-phenylglycine, β-aminobutyric acid, β-aminoisobutyric acid, (2-bromophenyl)glycine, (2-methoxyphenyl)glycine, (2-methylphenyl)glycine, (2-thiazoyl)glycine, (2-thienyl)glycine, 2-amino-3-(dimethylamino)-propionic acid, L-α,β-diaminopropionic acid, L-α-aminobutyric acid, L-α-t-butylglycine, L-(3-thienyl)glycine, L-2-amino-3-(dimethylamino)-propionic acid, L-2-aminocaproic acid dicyclohexyl-ammonium salt, L-2-indanylglycine, L-allylglycine.dicyclohexyl ammonium salt, L-cyclohexylglycine, L-phenylglycine, L-propargylglycine, L-norvaline, N-α-aminomethyl-L-alanine, D-α,γ-diaminobutyric acid, L-α,γ-diaminobutyric acid, β-cyclopropyl-L-alanine, (N-β-(2,4-dinitrophenyl))-L-α,β-diaminopropionic acid, (N-β-1-(4,4-dimethyl-2,6-dioxocyclohex-1-ylidene)ethyl)-D-α,β-diaminopropionic acid, (N-β-1-(4,4-dimethyl-2,6-dioxocyclohex-1-ylidene)ethyl)-L-α,β-diaminopropionic acid, (N-β-4-methyltrityl)-L-α,β-diaminopropionic acid, (N-β-allyloxycarbonyl)-L-α,β-diaminopropionic acid, (N-γ-1-(4,4-dimethyl-2,6-dioxocyclohex-1-ylidene)ethyl)-D-α,γ-diaminobutyric acid, (N-γ-1-(4,4-dimethyl-2,6-dioxocyclohex-1-ylidene)ethyl)-L-α,γ-diaminobutyric acid, (N-γ-4-methyltrityl)-D-α,γ-diaminobutyric acid, (N-γ-4-methyltrityl)-L-α,γ-diaminobutyric acid, (N-γ-allyloxycarbonyl)-L-α,γ-diaminobutyric acid, D-α, γ-diaminobutyric acid, 4,5-dehydro-L-leucine, cyclopentyl-D-Gly-OH, cyclopentyl-Gly-OH, D-allylglycine, D-homocyclohexylalanine, L-1-pyrenylalanine, L-2-aminocaproic acid, L-allylglycine, L-homocyclohexylalanine, and N-(2-hydroxy-4-methoxy-Bzl)-Gly-OH.

Exemplary amino acid analogs of arginine and lysine include, but are not limited to, citrulline, L-2-amino-3-guanidinopropionic acid, L-2-amino-3-ureidopropionic acid, L-citrulline, Lys(Me)₂—OH, Lys(N₃)—OH, Nδ-benzyloxycarbonyl-L-ornithine, Nω-nitro-D-arginine, Nω-nitro-L-arginine, α-methyl-ornithine, 2,6-diaminoheptanedioic acid, L-ornithine, (Nδ-1-(4,4-dimethyl-2,6-dioxo-cyclohex-1-ylidene)ethyl)-D-ornithine, (Nδ-1-(4,4-dimethyl-2,6-dioxo-cyclohex-1-ylidene)ethyl)-L-ornithine, (Nδ-4-methyltrityl)-D-ornithine, (Nδ-4-methyltrityl)-L-ornithine, D-ornithine, L-ornithine, Arg(Me)(Pbf)-OH, Arg(Me)₂-OH (asymmetrical), Arg(Me)2-OH (symmetrical), Lys(ivDde)-OH, Lys(Me)2-OH.HCl, Lys(Me3)-OH chloride, Nω-nitro-D-arginine, and Nω-nitro-L-arginine.

Exemplary amino acid analogs of aspartic and glutamic acids include, but are not limited to, α-methyl-D-aspartic acid, α-methyl-glutamic acid, α-methyl-L-aspartic acid, γ-methylene-glutamic acid, (N-γ-ethyl)-L-glutamine, [N-α-(4-aminobenzoyl)]-L-glutamic acid, 2,6-diaminopimelic acid, L-α-aminosuberic acid, D-2-aminoadipic acid, D-α-aminosuberic acid, α-aminopimelic acid, iminodiacetic acid, L-2-aminoadipic acid, threo-β-methyl-aspartic acid, γ-carboxy-D-glutamic acid γ,γ-di-t-butyl ester, γ-carboxy-L-glutamic acid γ,γ-di-t-butyl ester, Glu(OAll)-OH, L-Asu(OtBu)-OH, and pyroglutamic acid.

Exemplary amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Exemplary amino acid analogs of phenylalanine and tyrosine include, but are not limited to, β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2,4,5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Exemplary amino acid analogs of proline include 3,4-dehydro-proline, 4-fluoro-proline, cis-4-hydroxy-proline, thiazolidine-2-carboxylic acid, and trans-4-fluoro-proline.

Exemplary amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Exemplary amino acid analogs of tryptophan include, but are not limited to, α-methyl-tryptophan, β-(3-benzothienyl)-D-alanine, β-(3-benzothienyl)-L-alanine, 1-methyl-tryptophan, 4-methyl-tryptophan, 5-benzyloxy-tryptophan, 5-bromo-tryptophan, 5-chloro-tryptophan, 5-fluoro-tryptophan, 5-hydroxy-tryptophan, 5-hydroxy-L-tryptophan, 5-methoxy-tryptophan, 5-methoxy-L-tryptophan, 5-methyl-tryptophan, 6-bromo-tryptophan, 6-chloro-D-tryptophan, 6-chloro-tryptophan, 6-fluoro-tryptophan, 6-methyl-tryptophan, 7-benzyloxy-tryptophan, 7-bromo-tryptophan, 7-methyl-tryptophan, D-1,2,3,4-tetrahydro-norharman-3-carboxylic acid, 6-methoxy-1,2,3,4-tetrahydronorharman-1-carboxylic acid, 7-azatryptophan, L-1,2,3,4-tetrahydro-norharman-3-carboxylic acid, 5-methoxy-2-methyl-tryptophan, and 6-chloro-L-tryptophan.

In some instances, an artificial nucleotide comprises, for example, modifications at one or more of ribose moiety, phosphate moiety, nucleoside moiety, or a combination thereof. In some instances, an artificial nucleotide comprises a nucleic acid with a modification at a 2′ hydroxyl group of the ribose moiety. In some cases, the modification is a 2′-O-methyl modification or a 2′-O-methoxyethyl (2′-O-MOE) modification. The 2′-O-methyl modification is added a methyl group to the 2′ hydroxyl group of the ribose moiety whereas the 2′O-methoxyethyl modification is added a methoxyethyl group to the 2′ hydroxyl group of the ribose moiety. In some cases, the 2′ hydroxyl group includes a 2′-O-aminopropyl sugar conformation which can involve an extended amine group comprising a propyl linker that binds the amine group to the 2′ oxygen. In some cases, the 2′ hydroxyl group includes a locked or bridged ribose conformation (e.g., locked nucleic acid or LNA) where the 4′ ribose position can also be involved. In this modification, the oxygen molecule bound at the 2′ carbon is linked to the 4′ carbon by a methylene group, thus forming a 2′-C,4′-C-oxy-methylene-linked bicyclic ribonucleotide monomer. In some cases, the 2′ hydroxyl group comprises ethylene nucleic acids (ENA) such as for example 2′-4′-ethylene-bridged nucleic acid, which locks the sugar conformation into a C3′-endo sugar puckering conformation. In additional cases, the 2′ hydroxyl group includes 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue further comprises a morpholino, a peptide nucleic acid (PNA), a methylphosphonate nucleotide, a thiolphosphonate nucleotide, 2′-fluoro N3-P5′-phosphoramidite, 1′, 5′-anhydrohexitol nucleic acid (HNA), or a combination thereof.

In some embodiments, a ligand described herein comprises a small molecule ligand-electrophile compound.

Small Molecule Ligand-Electrophile Compounds

In some embodiments, a ligand-electrophile compound described herein is a small molecule compound that has a structure represented by Formula (II):

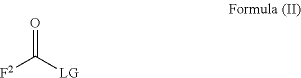

wherein,

F² is a small molecule fragment moiety; and

LG is a leaving group moiety.

In some embodiments, F² comprises C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, or a substituted or unsubstituted heteroaryl.

In some instances, a small molecule ligand-electrophile compound of Formula (I) has a structure selected from:

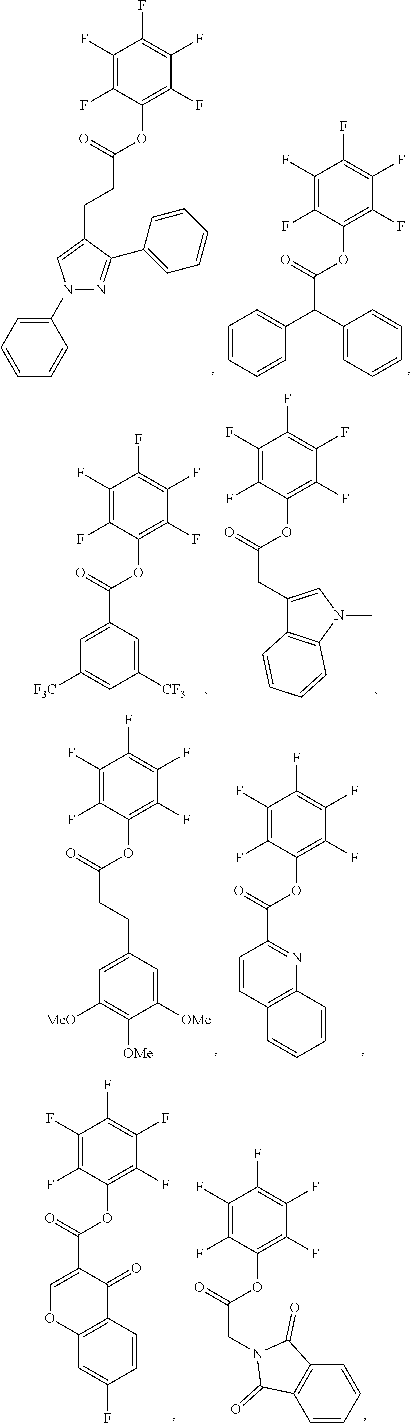

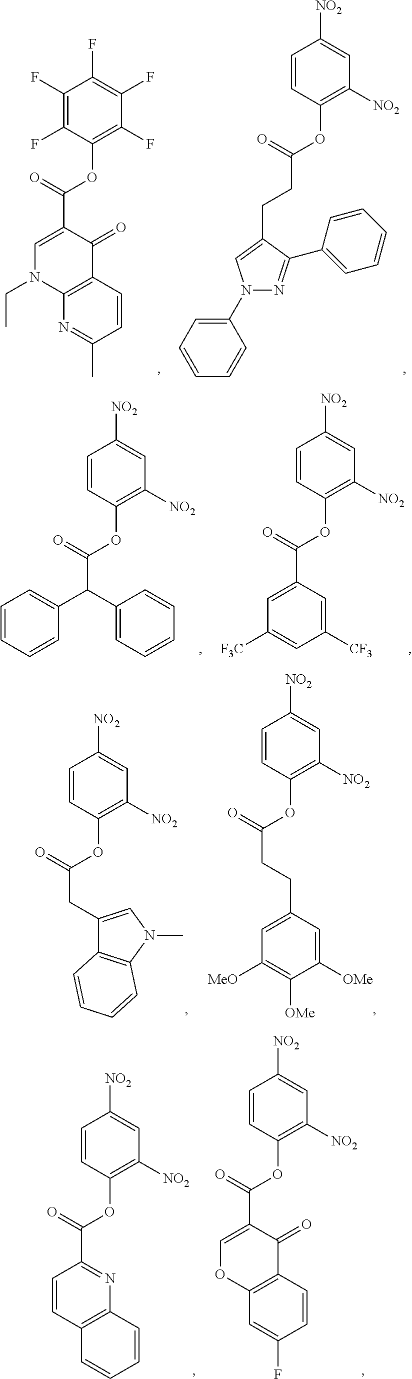

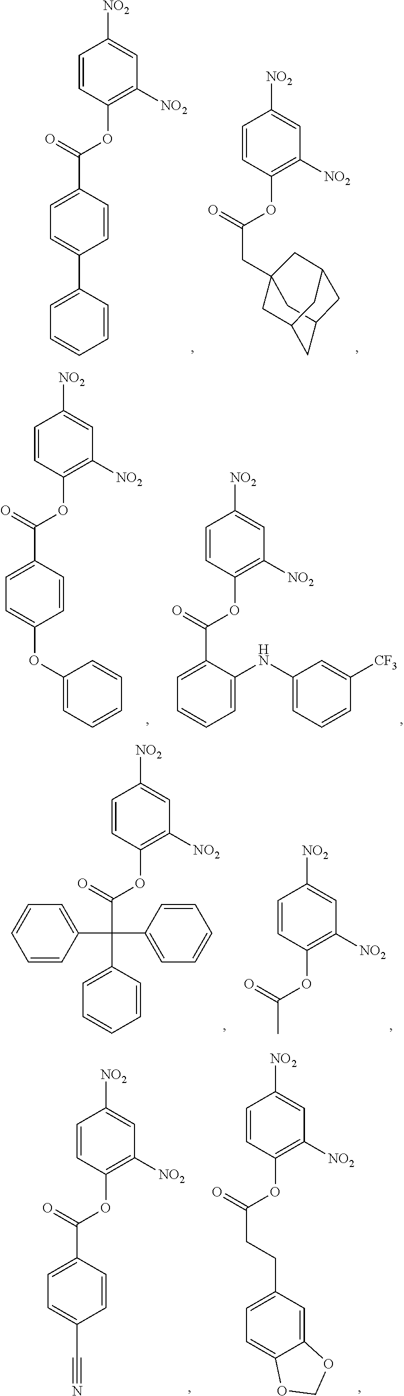

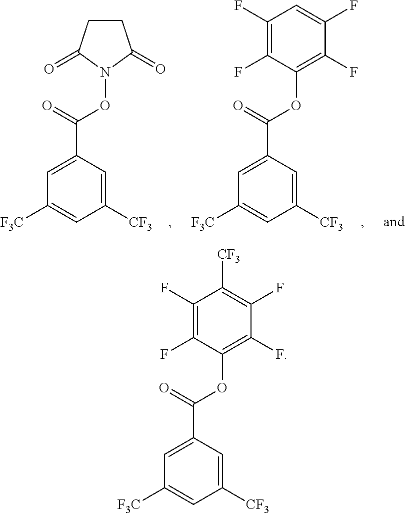

In some embodiments, F² comprises one or more —C(═O)LG moieties.

In some embodiments, the ligand-electrophile compound has a structure selected from:

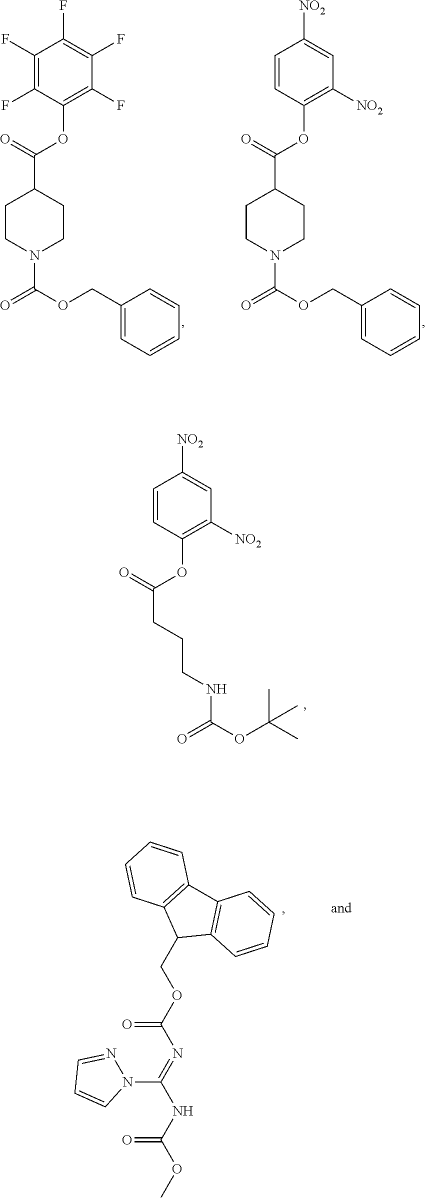 [SMILES: O=C(Oc1c(F)c(F)c(F)c(F)c1F)C1CCN(C(=O)OCc2ccccc2)CC1 ; O=C(Oc1ccc([N+](=O)[O-])cc1[N+](=O)[O-])C1CCN(C(=O)OCc2ccccc2)CC1 ; CC(C)(C)OC(=O)NCCCC(=O)Oc1ccc([N+](=O)[O-])cc1[N+](=O)[O-] ; COC(=O)N/C(=N/C(=O)OCC1c2ccccc2-c2ccccc21)n1cccn1]

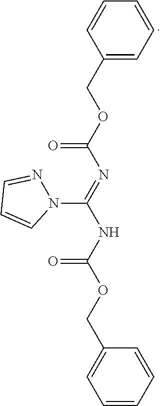 [SMILES: O=C(/N=C(/NC(=O)OCc1ccccc1)n1cccn1)OCc1ccccc1]

In some cases, F² is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

Often, a ligand-electrophile is a non-naturally occurring compound. In some instances, reaction of a ligand-electrophile with the amino group of a lysine-containing protein results in non-naturally occurring product. In some instances, the amino group of the lysine-containing protein is connected to a small molecule fragment moiety via an amide bond after reaction with a ligand-electrophile.

Further Forms of Compounds

In one aspect, the compound of Formula (I), possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein might be conveniently prepared or formed during the processes described herein. In addition, the compounds provided herein might exist in unsolvated as well as solvated forms. In general, the solvated forms are considered equivalent to the unsolvated forms for the purposes of the compounds and methods provided herein.

Compound Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “oxo” refers to the ═O substituent.

The term “thioxo” refers to the ═S substituent.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₅ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₅ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aromatic” refers to a planar ring having a delocalized π-electron system containing 4n+2 π electrons, where n is an integer. Aromatics might be optionally substituted. The term “aromatic” includes both aryl groups (e.g., phenyl, naphthalenyl) and heteroaryl groups (e.g., pyridinyl, quinolinyl).

The terms “carbocyclic” or “carbocycle” refer to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “aryl” refers to an aromatic ring wherein each of the atoms forming the ring is a carbon atom. Aryl groups might be optionally substituted. Examples of aryl groups include, but are not limited to phenyl, and naphthyl. In some embodiments, the aryl is phenyl. Depending on the structure, an aryl group might be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic, fused, or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the monocyclic cyclcoalkyl is cyclopentyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “bridged” refers to any ring structure with two or more rings that contains a bridge connecting two bridgehead atoms. The bridgehead atoms are defined as atoms that are the part of the skeletal framework of the molecule and which are bonded to three or more other skeletal atoms. In some embodiments, the bridgehead atoms are C, N, or P. In some embodiments, the bridge is a single atom or a chain of atoms that connects two bridgehead atoms. In some embodiments, the bridge is a valence bond that connects two bridgehead atoms. In some embodiments, the bridged ring system is cycloalkyl. In some embodiments, the bridged ring system is heterocycloalkyl.

The term “fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with one or more N, S, and O atoms. The non-limiting examples of fused heterocyclyl or heteroaryl ring structures include 6-5 fused heterocycle, 6-6 fused heterocycle, 5-6 fused heterocycle, 5-5 fused heterocycle, 7-5 fused heterocycle, and 5-7 fused heterocycle.

The term “halo” or “halogen” refers to bromo, chloro, fluoro or iodo.

The term “haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

The term “haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “fluorocycloalkyl” refers to a cycloalkyl in which one or more hydrogen atoms are replaced by a fluorine atom. In one aspect, a fluorocycloalkyl is a C₁-C₆fluorocycloalkyl. In some embodiments, a fluorocycloalkyl is selected from 2,2-difluorocyclopropyl, heptafluorocyclobutyl, 1-fluorocyclopentyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆heteroalkyl. Representative heteroalkyl groups include, but are not limited to —OCH₂OMe, —OCH₂CH₂OH, —OCH₂CH₂OMe, or —OCH₂CH₂OCH₂CH₂NH₂.

The term “heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1 (2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl. In some embodiments, a heteroaryl group is partially reduced to form a heterocycloalkyl group defined herein. In some embodiments, a heteroaryl group is fully reduced to form a heterocycloalkyl group defined herein.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl)-CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). In some embodiments, an optional substituent on an aliphatic carbon atom (acyclic or cyclic) includes thioxo (═S).

The term “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

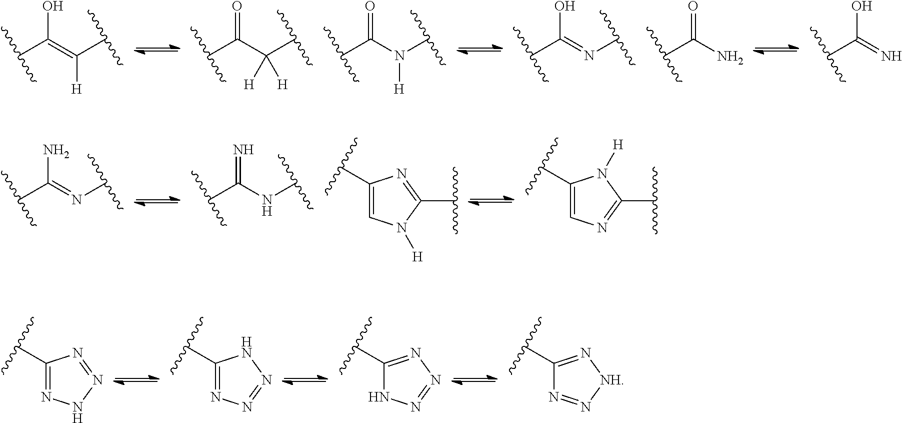

Lysine-Containing Proteins

In some embodiments, disclosed herein are lysine-containing proteins that comprises one or more ligandable lysines. In some instances, the lysine-containing protein is a soluble protein. In other instances, the lysine-containing protein is a membrane protein. In some cases, the lysine-containing protein is involved in one or more of a biological process such as protein transport, lipid metabolism, apoptosis, transcription, electron transport, mRNA processing, or host-virus interaction. In additional cases, the lysine-containing protein is associated with one or more of diseases such as cancer or one or more disorders or conditions such as immune, metabolic, developmental, reproductive, neurological, psychiatric, renal, cardiovascular, or hematological disorders or conditions.

In some instances, a ligandable lysine residue is located from 10 Å to 60 Å away from an active site residue. In some instances, a ligandable lysine residue is located at least 10 Å, 12 Å, 15 Å, 20 Å, 25 Å, 30 Å, 35 Å, 40 Å, 45 Å, or 50 Å away from an active site residue. In some instances, a ligandable lysine residue is located about 10 Å, 12 Å, 15 Å, 20 Å, 25 Å, 30 Å, 35 Å, 40 Å, 45 Å, or 50 Å away from an active site residue.

In some cases, the lysine-containing protein exists in an active form. In additional cases, the lysine-containing protein exists in a pro-active form.

In some embodiments, the lysine-containing protein comprises one or more functions of an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the lysine-containing protein is an enzyme, a transporter, a receptor, a channel protein, an adaptor protein, a scaffolding protein, a modulator, a chaperone, a signaling protein, a plasma protein, transcription related protein, translation related protein, mitochondrial protein, or cytoskeleton related protein. In some instances, the lysine-containing protein has an uncategorized function.

In some embodiments, the lysine-containing protein is an enzyme. An enzyme is a protein molecule that accelerates or catalyzes chemical reaction. In some embodiments, non-limiting examples of enzymes include kinases, proteases, or deubiquitinating enzymes.

In some instances, exemplary kinases include tyrosine kinases such as the TEC family of kinases such as Tec, Bruton's tyrosine kinase (Btk), interleukin-2-indicible T-cell kinase (Itk) (or Emt/Tsk), Bmx, and Txk/Rlk; spleen tyrosine kinase (Syk) family such as SYK and Zeta-chain-associated protein kinase 70 (ZAP-70); Src kinases such as Src, Yes, Fyn, Fgr, Lck, Hck, Blk, Lyn, and Frk; JAK kinases such as Janus kinase 1 (JAK1), Janus kinase 2 (JAK2), Janus kinase 3 (JAK3), and Tyrosine kinase 2 (TYK2); or ErbB family of kinases such as Her1 (EGFR, ErbB1), Her2 (Neu, ErbB2), Her3 (ErbB3), and Her4 (ErbB4).

In some embodiments, the lysine-containing protein is a protease. In some embodiments, the protease is a cysteine protease. In some cases, the cysteine protease is a caspase. In some instances, the caspase is an initiator (apical) caspase. In some instances, the caspase is an effector (executioner) caspase. Exemplary caspase includes CASP2, CASP8, CASP9, CASP10, CASP3, CASP6, CASP7, CASP4, and CASP5. In some instances, the cysteine protease is a cathepsin. Exemplary cathepsin includes Cathepsin B, Cathepsin C, Cathepsin F, Cathepsin H, Cathepsin K, Cathepsin L1, Cathepsin L2, Cathepsin O, Cathepsin S, Cathepsin W, or Cathepsin Z.

In some embodiments, the lysine-containing protein is a deubiquitinating enzyme (DUB). In some embodiments, exemplary deubiquitinating enzymes include cysteine proteases DUBs or metalloproteases. Exemplary cysteine protease DUBs include ubiquitin-specific protease (USP/UBP) such as USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46; ovarian tumor (OTU) proteases such as OTUB1 and OTUB2; Machado-Josephin domain (MJD) proteases such as ATXN3 and ATXN3L; and ubiquitin C-terminal hydrolase (UCH) proteases such as BAP1, UCHL1, UCHL3, and UCHL5. Exemplary metalloproteases include the Jabl/Mov34/Mprl Padl N-terminal+(MPN+) (JAMM) domain proteases.

In some embodiments, exemplary lysine-containing proteins as enzymes include, but are not limited to, Abhydrolase domain-containing protein 10, mitochondrial (ABHD10); Adenosine kinase (ADK); Aldo-keto reductase family 1 member C3 (AKR1C3); Bis(5-nucleo syl)-tetrapho sphatase (NUDT2); C-1-tetrahydrofolate synthase, cytoplasmic (MTHFD 1); CCR4-NOT transcription complex subunit 4 (CNOT4); Coproporphyrinogen-III oxidase, mitochondrial (CPOX); Cyclin-dependent kinase 2 (CDK2); Delta(3,5)-Delta(2,4)-dienoyl-CoA isomerase, mitochondrial (ECH1); DNA (cytosine-5)-methyltransferase 1 (DNMT1); DNA-directed RNA polymerases I, II, and III subunit (POLR2L); Dual specificity mitogen-activated protein kinase (MAP2K3); Electron transfer flavoprotein subunit alpha, mitochondrial (ETFA); Elongation factor 1-gamma (EEF1G); Endoplasmic reticulum aminopeptidase 1 (ERAP1); Enolase-phosphatase E1 (ENOPHI); ERO1-like protein alpha (ERO1L); Ferrochelatase, mitochondrial (FECH); Fumarate hydratase, mitochondrial (FH); Fumarylacetoacetase (FAH); GDP-L-fucose synthase (TSTA3); Glucose-6-phosphate 1-dehydrogenase (G6PD); Glutamate dehydrogenase 1, mitochondrial (GLUD1); Glutathione S-transferase theta-2B (GSTT2B); Haloacid dehalogenase-like hydrolase domain-containing 3 (HDHD3); Hexokinase-1 (HK1); Inosine-5-monophosphate dehydrogenase 1 (IMPDH1); Isocitrate dehydrogenase (IDH3B); L-lactate dehydrogenase B chain (LDHB); Mitochondrial ribonuclease P protein 1 (TRMT10C); Mitogen-activated protein kinase kinase kinase kinase (MAP4K5); Neurolysin, mitochondrial (NLN); Nucleoside diphosphate-linked moiety X motif 22 (NUDT22); 5-nucleotidase domain-containing protein 1 (NT5DC1); Ornithine aminotransferase, mitochondrial (OAT); 6-phosphofructokinase, liver type (PFKL); 6-phosphofructokinase, muscle type (PFKM); 6-phosphofructokinase type C (PFKP); Prostaglandin reductase 1 (PTGR1); Puromycin-sensitive aminopeptidase (NPEPPS); Pyridoxine-5-phosphate oxidase (PNPO); Serine/threonine-protein kinase mTOR (MTOR); Sphingomyelin phosphodiesterase (SMPD1); SUMO-activating enzyme subunit 2 (UBA2); Superoxide dismutase (SOD2); Thiopurine S-methyltransferase (TPMT); Thymidylate kinase (DTYMK); Tryptophan—tRNA ligase, cytoplasmic (WARS); Ubiquitin carboxyl-terminal hydrolase isozyme L5 (UCHL5); Ubiquitin-like modifier-activating enzyme 6 (UBA6); or X-ray repair cross-complementing protein 6 (XRCC6).

In some embodiments, the lysine-containing protein is a signaling protein. In some instances, exemplary signaling protein includes vascular endothelial growth factor (VEGF) proteins or proteins involved in redox signaling. Exemplary VEGF proteins include VEGF-A, VEGF-B, VEGF-C, VEGF-D, and PGF. Exemplary proteins involved in redox signaling include redox-regulatory protein FAM213A.

In some embodiments, the lysine-containing protein is a channel, transporter or receptor. Exemplary lysine-containing proteins as channels, transporters, or receptors include, but are not limited to, AP-1 complex subunit gamma-1 (AP1G1); Importin subunit alpha-2 (KPNA2); Sideroflexin-1 (SFXN1); or V-type proton ATPase subunit F (ATP6V1F).

In some embodiments, the lysine-containing protein is a chaperone. Exemplary lysine-containing proteins as chaperones include, but are not limited to, 60 kDa heat shock protein (mitochondrial) (HSPD1), T-complex protein 1 subunit eta (CCT7), T-complex protein 1 subunit epsilon (CCT5), Heat shock 70 kDa protein 4 (HSPA4), GrpE protein homolog 1 (mitochondrial) (GRPEL1), Tubulin-specific chaperone E (TBCE), Protein unc-45 homolog A (UNC45A), Serpin H1 (SERPINH1), Tubulin-specific chaperone D (TBCD), Peroxisomal biogenesis factor 19 (PEX19), BAG family molecular chaperone regulator 5 (BAG5), T-complex protein 1 subunit theta (CCT8), Protein canopy homolog 3 (CNPY3), DnaJ homolog subfamily C member 10 (DNAJC10), ATP-dependent Clp protease ATP-binding subunit clp (CLPX), or Midasin (MDN1).

In some embodiments, the lysine-containing protein is an adapter, scaffolding or modulator protein. Exemplary lysine-containing proteins as adapter, scaffolding, or modulator proteins include, but are not limited to, 26S proteasome non-ATPase regulatory subunit 10 (PSMD10); 26S proteasome non-ATPase regulatory subunit 11 (PSMD11); 39S ribosomal protein L53, mitochondrial (MRPL53); 78 kDa glucose-regulated protein (HSPA5); Actin-related protein 2 (ACTR2); Adenylyl cyclase-associated protein 1 (CAP1); ADP/ATP translocase 1 (SLC25A4); ADP/ATP translocase 2 (SLC25A5); ADP/ATP translocase 3 (SLC25A6); ADP-ribosylation factor-like protein 6-interacting protein 1 (ARL6IP1); Alpha-taxilin (TXLNA); Angio-associated migratory cell protein (AAMP); Arfaptin-1 (ARFIP1); AP-3 complex subunit beta-1 (AP3B1); Apoptosis regulator BAX (BAX); Astrocytic phosphoprotein PEA-15 (PEA15); ATP-binding cassette sub-family E member 1 (ABCE1); ATPase inhibitor, mitochondrial (ATPIF1); B-cell receptor-associated protein 31 (BCAP31); Beta-catenin-like protein 1 (CTNNBL1); BH3-interacting domain death agonist (BID); cAMP-regulated phosphoprotein 19 (ARPP19); Calcyclin-binding protein (CACYBP); Calponin-2 (CNN2); Calponin-3 (CNN3); Charged multivesicular body protein 5 (CHMP5); COMM domain-containing protein 2 (COMMD2); COMM domain-containing protein 4 (COMMD4); CD166 antigen (ALCAM); COP9 signalosome complex subunit 1 (GPS1); Coronin-1B (CORO1B); Coronin-1C (CORO1C); Cullin-2 (CUL2); Cullin-3 (CUL3); Cyclin-A2 (CCNA2); Destrin (DSTN); DnaJ homolog subfamily C member 3 (DNAJC3); DnaJ homolog subfamily C member 9 (DNAJC9); Dynactin subunit 2 (DCTN2); EH domain-containing protein 1 (EHD1); Endophilin-A2 (SH3GL1); Endoplasmic reticulum resident protein 29 (ERP29); Endoplasmin (HSP90B1); Epididymal secretory protein E1 (NPC2); Ezrin (EZR); F-actin-capping protein subunit alpha-1 (CAPZA1); F-actin-capping protein subunit alpha-2 (CAPZA2); Filamin-C (FLNC); Galectin-1 (LGALS 1); Gamma-aminobutyric acid receptor-associated protein (GABARAPL2); Glutamate-cysteine ligase regulatory subunit (GCLM); Golgi resident protein GCP60 (ACBD3); Golgi phosphoprotein 3 (GOLPH3); GrpE protein homolog 1, mitochondrial (GRPEL1); GTP-binding protein Rheb (RHEB); Hypoxia up-regulated protein 1 (HYOU1); KIF1-binding protein (KIAA1279); Septin-1 (SEPT1); Leucine-rich repeat protein SHOC-2 (SHOC2); Leucine-rich repeat-containing protein 20 (LRRC20); Leucine zipper transcription factor-like protein 1 (LZTFL1); LIM and senescent cell antigen-like-containing domain protein 1 (LIMS 1); Mediator of RNA polymerase II transcription subunit (MED28); Microtubule-actin cross-linking factor 1, isoforms 1/2/3/5 (MACF1); Microtubule-associated proteins 1A/1B light chain (MAP1LC3B); Mitochondrial carrier homolog 2 (MTCH2); Mitochondrial translocator assembly and maintenance protein 41 homolog (TAMM41); Mitochondrial import receptor subunit TOM34 (TOMM34); Mitochondrial import inner membrane translocase subunit TIM14 (DNAJC19); Mixed lineage kinase domain-like protein (MLKL); Myosin regulatory light chain 12B (MYL12B); Nuclear autoantigenic sperm protein (NASP); N-alpha-acetyltransferase 25, NatB auxiliary subunit (NAA25); Nuclear pore complex protein Nup205 (NUP205); Nucleoporin NUP188 homolog (NUP188); Nucleoporin SEH1 (SEH1L); Nuclear autoantigenic sperm protein (NASP); Perilipin-3 (PLIN3); Plasminogen activator inhibitor 1 (SERPINE1); Pleckstrin homology-like domain family A member 1 (PHLDA1); Prefoldin subunit 2 (PFDN2); Prefoldin subunit 5 (PFDN5); Programmed cell death 6-interacting protein (PDCD6IP); Protein kinase C and casein kinase substrate in neurons protein 2 (PACSIN2); Protein S100-A11 (S100A11); Protein NipSnap homolog 2 (GBAS); Protein NipSnap homolog 3A (NIPSNAP3A); Protein sel-1 homolog 1 (SEL1L); Proactivator polypeptide (PSAP); Programmed cell death 6-interacting protein (PDCD6IP); Programmed cell death protein 10 (PDCD10); Prefoldin subunit 2 (PFDN2); Prefoldin subunit 3 (VBP1); Prelamin-A/C (LMNA); Proteasome activator complex subunit 3 (PSME3); RAD50-interacting protein 1 (RINT1); Rapl GTPase-GDP dissociation stimulator 1 (RAP1GDS1); Ras GTPase-activating-like protein IQGAP1 (IQGAP1); Ras-related protein Rab-10 (RAB10); Ras-related protein Rab-13 (RAB13); Ras-related protein Rab-34 (RAB34); Rab3 GTPase-activating protein catalytic subunit (RAB3GAP1); Ras GTPase-activating-like protein IQGAP1 (IQGAP1); Reticulon-3 (RTN3); Rho GDP-dissociation inhibitor 2 (ARHGDIB); Rho guanine nucleotide exchange factor 12 (ARHGEF12); Secl family domain-containing protein 1 (SCFD1); Sell repeat-containing protein 1 (SELRC1); Serpin H1 (SERPINH1); Septin-6 (SEPT6); Septin-7 (SEPT7); Small glutamine-rich tetratricopeptide repeat-containing protein alpha (SGTA); Sorting nexin-3 (SNX3); Sorting nexin-8 (SNX8); Spastin (SPAST); Spectrin alpha chain, non-erythrocytic 1 (SPTAN1); Stathmin (STMN1); Stromal interaction molecule 1 (STIM1); Striatin-3 (STRN3); Structural maintenance of chromosomes protein 2 (SMC2); Talin-1 (TLN1); T-complex protein 1 subunit beta (CCT2); T-complex protein 1 subunit gamma (CCT3); T-complex protein 1 subunit theta (CCT8); Torsin-1A-interacting protein 2 (TOR1AIP2); Trafficking protein particle complex subunit 5 (TRAPPC5); Transmembrane emp24 domain-containing protein 5 (TMED5); Transmembrane emp24 domain-containing protein 9 (TMED9); Transforming acidic coiled-coil-containing protein (TACC3); Translational activator of cytochrome c oxidase 1 (TACO 1); Transthyretin (TTR); Tubulin alpha-4A chain (TUBA4A); Tubulin-specific chaperone E (TBCE); Twinfilin-1 (TWF1); Vacuolar protein sorting-associated protein VTA1 homolog (VTA1); Vasodilator-stimulated phosphoprotein (VASP); Vesicle-associated membrane protein-associated protein A (VAPA); Voltage-dependent anion-selective channel protein (VDAC3); or UPF0366 protein C11orf67 (C11orf67).

In some embodiments, the lysine-containing protein is transcription related protein or translation related protein. In some instances, the lysine-containing protein is involved in gene expression, replication, and/or nucleic acid binding. Exemplary lysine-containing proteins include, but are not limited to, 26S protease regulatory subunit 10B (PSMC6); 28S ribosomal protein S24, mitochondrial (MRPS24); 39S ribosomal protein L12, mitochondrial (MRPL12); 40S ribosomal protein S10 (RPS10); 60S ribosomal protein L7-like 1 (RPL7L1); 60S ribosomal protein L9 (RPL9P9); 60S ribosomal protein L10 (RPL10); Apoptotic chromatin condensation inducer in the nucleus (ACIN1); Arf-GAP domain and FG repeat-containing protein 1 (AGFG1); Bcl-2-associated transcription factor 1 (BCLAF1); Cell differentiation protein RCD1 homolog (RQCD1); Chromatin accessibility complex protein 1 (CHRAC1); Constitutive coactivator of PPAR-gamma-like protein 1 (FAM120A); Cysteine and glycine-rich protein 2 (CSRP2); Cytoplasmic dynein 1 heavy chain 1 (DYNC1H1); DBIRD complex subunit KIAA1967 (KIAA1967); DNA damage-binding protein 1 (DDB 1); ELAV-like protein 1 (ELAVL1); Elongation factor 1-alpha 1 (EEF1A1); Elongation factor 2 (EEF2); Eukaryotic translation initiation factor 3 subunit (EIF3G); Eukaryotic translation initiation factor 3 subunit (EIF3L); Eukaryotic translation initiation factor 5A-1-like (EIF5AL1); Eukaryotic translation initiation factor 5A-2 (EIF5A2); Far upstream element-binding protein 1 (FUBP1); Far upstream element-binding protein 2 (KHSRP); Far upstream element-binding protein 3 (FUBP3); Gamma-aminobutyric acid receptor-associated protein-like 1 (GABARAPL1); Golgin subfamily B member 1 (GOLGB 1); G-rich sequence factor (GRSF1); Heat shock protein 75 kDa, mitochondrial (TRAP1); HAUS augmin-like complex subunit 4 (HAUS4); Heterogeneous nuclear ribonucleoprotein A/B (HNRNPAB); Heterogeneous nuclear ribonucleoprotein K (HNRNPK); Histone H3.3C (H3F3C); Interferon-induced protein with tetratricopeptide (IFIT3); Interleukin enhancer-binding factor 2 (ILF2); Interleukin enhancer-binding factor 3 (ILF3); Kinesin-like protein KIF2C (KIF2C); Leucine-rich repeat-containing protein 59 (LRRC59); Microtubule-associated protein RP/EB family member (MAPRE1); Muscleblind-like protein 1 (MBNL1); Neuroblast differentiation-associated protein AHNA (AHNAK); Non-POU domain-containing octamer-binding protein (NONO); Nuclear pore complex protein Nup50 (NUP50); Obg-like ATPase 1 (OLA1); Paired amphipathic helix protein Sin3a (SIN3A); Plectin (PLEC); Poly(U)-binding-splicing factor PUF60 (PUF60); Polymerase I and transcript release factor (PTRF); Probable ATP-dependent RNA helicase DDX20 (DDX20); Protein mago nashi homolog 2 (MAGOHB); Reticulon-4 (RTN4); Ribonuclease H2 subunit C (RNASEH2C); Ribosome-binding protein 1 (RRBP1); RNA-binding protein 14 (RBM14); RuvB-like 2 (RUVBL2); Signal recognition particle 54 kDa protein (SRP54); Splicing factor 1 (SF1); Splicing factor 3A subunit 1 (SF3A1); Splicing factor 3A subunit 3 (SF3A3); SRA stem-loop-interacting RNA-binding protein, mitochondrial (SLIRP); TAR DNA-binding protein 43 (TARDBP); THO complex subunit 4 (ALYREF); or Tumor protein D54 (TPD52L2).

In some embodiments, a lysine-containing protein comprises a protein illustrated in Tables 1-3. In some instances, a lysine-containing protein comprises a protein illustrated in Table 1. In some embodiments, the lysine-containing protein comprises a lysine residue denoted in Table 1. In some instances, a lysine-containing protein comprises a protein illustrated in Table 2. In some embodiments, the lysine-containing protein comprises a lysine residue denoted in Table 2. In some instances, a lysine-containing protein comprises a protein illustrated in Table 3. In some embodiments, the lysine-containing protein comprises a lysine residue denoted in Table 3.

In some embodiments, disclosed herein is a modified lysine-containing protein which comprises a small molecule fragment moiety, covalently bonded to a lysine residue of a lysine-containing protein. In some instances, the lysine-containing protein is selected from Table 1. In other instances, the lysine-containing protein is selected from Table 2. In some cases, the lysine-containing protein is selected from an enzyme; a protein involved in gene expression, replication, and/or nucleic acid binding; or a protein involved in scaffolding, modulator, and/or adaptor function. In some cases, the covalent bond is formed by reaction with a non-naturally occurring small molecule probe having a structure of Formula (I):

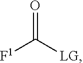

wherein F¹ is a small molecule fragment moiety comprising an alkyne moiety, a fluorophore moiety, a labeling group, or a combination thereof; and LG is a leaving group moiety. In some cases, the covalent bond is formed by reaction with a non-naturally occurring ligand-electrophile having a structure of Formula (II):

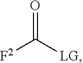

wherein F² is a small molecule fragment moiety; and LG is a leaving group moiety.

In some embodiments, one or more enzymes are modified and the modified enzymes each independently comprise a small molecule fragment moiety, covalently bonded to a lysine residue of an enzyme. In some instances, the one or more enzymes comprise E3 ubiquitin-protein ligase ARIH2 (ARIH2), Copine-3 (CPNE3), Cullin-1 (CUL1), Glucose-6-phosphate 1-dehydrogenase (G6PD), E3 ubiquitin-protein ligase HUWE1 (HUWE1), E3 SUMO-protein ligase NSE2 (NSMCE2), Bis(5-nucleosyl)-tetraphosphatase (NUDT2), 6-phosphofructokinase type C (PFKP), Pyridoxine-5-phosphate oxidase (PNPO), Proteasome subunit alpha type-6 (PSMA6), E3 ubiquitin-protein ligase RBX1 (RBX1), E3 ubiquitin-protein ligase BRE1B (RNF40), E3 ubiquitin/ISG15 ligase TRIM25 (TRIM25), Transcription intermediary factor 1-beta (TRIM28), Ubiquitin-like modifier-activating enzyme 1 (UBA1), Ubiquitin-like modifier-activating enzyme 5 (UBA5), Ubiquitin-like modifier-activating enzyme 6 (UBA6), Ubiquitin-conjugating enzyme E2 D2 (UBE2D2), Ubiquitin-conjugating enzyme E2 G2 (UBE2G2), SUMO-conjugating enzyme UBC9 (UBE2I), Ubiquitin-conjugating enzyme E2 (UBE2K), Ubiquitin-conjugating enzyme E2 L3 (UBE2L3), Ubiquitin-conjugating enzyme E2 N (UBE2N), Ubiquitin-conjugating enzyme E2 S (UBE2S), Ubiquitin-conjugating enzyme E2 variant 1 (UBE2V1), Ubiquitin-conjugating enzyme E2 (UBE2Z), Ubiquitin-like protein 4A (UBL4A), Ubiquitin-like domain-containing CTD phosphatase 1 (UBLCP1), Ubiquitin carboxyl-terminal hydrolase isozyme L1 (UCHL1), Ubiquitin carboxyl-terminal hydrolase isozyme L5 (UCHL5), Ubiquitin carboxyl-terminal hydrolase 11 (USP11), Ubiquitin carboxyl-terminal hydrolase 14 (USP14), or any combinations thereof. In some cases, the modified enzyme is E3 ubiquitin-protein ligase ARIH2 (ARIH2) and the site of modification comprises K460, wherein the residue position corresponds to K460 of UniProtKB accession number 095376. In some cases, the modified enzyme is Copine-3 (CPNE3) and the site of modification comprises K390 or K500, wherein the residue positions correspond to K390 and K500 of UniProtKB accession number O75131. In some cases, the modified enzyme is Cullin-1 (CUL1) and the site of modification comprises K708, wherein the residue position corresponds to K708 of UniProtKB accession number Q13616. In some cases, the modified enzyme is Glucose-6-phosphate 1-dehydrogenase (G6PD) and the site of modification comprises K171, K205, K408, or K497, wherein the residue positions correspond to K171, K205, K408, and K497 of UniProtKB accession number P11413. In some cases, the modified enzyme is E3 ubiquitin-protein ligase HUWE1 (HUWE1) and the site of modification comprises K3345, wherein the residue position corresponds to K3345 of UniProtKB accession number Q7Z6Z7. In some cases, the modified enzyme is E3 SUMO-protein ligase NSE2 (NSMCE2) and the site of modification comprises K107, wherein the residue position corresponds to K107 of UniProtKB accession number Q96MF7. In some cases, the modified enzyme is Bis(5-nucleosyl)-tetraphosphatase (NUDT2) and the site of modification comprises K89, wherein the residue position corresponds to K89 of UniProtKB accession number P50583. In some cases, the modified enzyme is 6-phosphofructokinase type C (PFKP) and the site of modification comprises K15, K109, K139, K395, K459, K486, K688, K736, or K759, wherein the residue positions correspond to K15, K109, K139, K395, K459, K486, K688, K736, and K759 of UniProtKB accession number Q01813. In some cases, the modified enzyme is Pyridoxine-5-phosphate oxidase (PNPO) and the site of modification comprises K100, wherein the residue position corresponds to K100 of UniProtKB accession number Q9NVS9. In some cases, the modified enzyme is Proteasome subunit alpha type-6 (PSMA6) and the site of modification comprises K104, wherein the residue position corresponds to K104 of UniProtKB accession number P60900. In some cases, the modified enzyme is E3 ubiquitin-protein ligase RBX1 (RBX1) and the site of modification comprises K105, wherein the residue position corresponds to K105 of UniProtKB accession number P62877. In some cases, the modified enzyme is E3 ubiquitin-protein ligase BRE1B (RNF40) and the site of modification comprises K420, wherein the residue position corresponds to K420 of UniProtKB accession number O75150. In some cases, the modified enzyme is E3 ubiquitin/ISG15 ligase TRIM25 (TRIM25) and the site of modification comprises K65, K237, K273, or K335, wherein the residue positions correspond to K65, K237, K273, and K335 of UniProtKB accession number Q14258. In some cases, the modified enzyme is Transcription intermediary factor 1-beta (TRIM28) and the site of modification comprises K254, K261, K296, K304, K337, K377, K407, K770, or K779, wherein the residue positions correspond to K254, K261, K296, K304, K337, K377, K407, K770, and K779 of UniProtKB accession number Q13263. In some cases, the modified enzyme is Ubiquitin-like modifier-activating enzyme 1 (UBA1) and the site of modification comprises K68, K416, K627, K635, K802, or K889, wherein the residue positions correspond to K68, K416, K627, K635, K802, and K889 of UniProtKB accession number P22314. In some cases, the modified enzyme is Ubiquitin-like modifier-activating enzyme 5 (UBA5) and the site of modification comprises K60, wherein the residue position corresponds to K60 of UniProtKB accession number Q9GZZ9. In some cases, the modified enzyme is Ubiquitin-like modifier-activating enzyme 6 (UBA6) and the site of modification comprises K86, wherein the residue position corresponds to K86 of UniProtKB accession number AOAVT1. In some cases, the modified enzyme is Ubiquitin-conjugating enzyme E2 D2 (UBE2D2) and the site of modification comprises K8, K101, or K144, wherein the residue positions correspond to K8, K101, and K144 of UniProtKB accession number P62837. In some cases, the modified enzyme is Ubiquitin-conjugating enzyme E2 G2 (UBE2G2) and the site of modification comprises K118, wherein the residue position corresponds to K118 of UniProtKB accession number P60604. In some cases, the modified enzyme is SUMO-conjugating enzyme UBC9 (UBE2I) and the site of modification comprises K18, K30, or K49, wherein the residue positions correspond to K18, K30, and K49 of UniProtKB accession number P63279. In some cases, the modified enzyme is Ubiquitin-conjugating enzyme E2 (UBE2K) and the site of modification comprises K164, wherein the residue position corresponds to K164 of UniProtKB accession number P61086. In some cases, the modified enzyme is Ubiquitin-conjugating enzyme E2 L3 (UBE2L3) and the site of modification comprises K100, K82, K9, or K64, wherein the residue positions correspond to K100, K82, K9, and K64 of UniProtKB accession number P68036. In some cases, the modified enzyme is Ubiquitin-conjugating enzyme E2 N (UBE2N) and the site of modification comprises K10, K68, K74, K82, or K92, wherein the residue position corresponds to K10, K68, K74, K82, and K92 of UniProtKB accession number P61088. In some cases, the modified enzyme is Ubiquitin-conjugating enzyme E2 S (UBE2S) and the site of modification comprises K197, wherein the residue position corresponds to K197 of UniProtKB accession number Q16763. In some cases, the modified enzyme is Ubiquitin-conjugating enzyme E2 variant 1 (UBE2V1) and the site of modification comprises K74 or K87, wherein the residue positions correspond to K74 and K87 of UniProtKB accession number Q13404. In some cases, the modified enzyme is Ubiquitin-conjugating enzyme E2 (UBE2Z) and the site of modification comprises K304, wherein the residue position corresponds to K304 of UniProtKB accession number Q9H832. In some cases, the modified enzyme is Ubiquitin-like protein 4A (UBL4A) and the site of modification comprises K101, wherein the residue position corresponds to K101 of UniProtKB accession number P11441. In some cases, the modified enzyme is Ubiquitin-like domain-containing CTD phosphatase 1 (UBLCP1) and the site of modification comprises K117, wherein the residue position corresponds to K117 of UniProtKB accession number Q8WVY7. In some cases, the modified enzyme is Ubiquitin carboxyl-terminal hydrolase isozyme L1 (UCHL1) and the site of modification comprises K4, wherein the residue position corresponds to K4 of UniProtKB accession number P09936. In some cases, the modified enzyme is Ubiquitin carboxyl-terminal hydrolase isozyme L5 (UCHL5) and the site of modification comprises K323, wherein the residue position corresponds to K323 of UniProtKB accession number Q9Y5K5. In some cases, the modified enzyme is Ubiquitin carboxyl-terminal hydrolase 11 (USP11) and the site of modification comprises K191 or K493, wherein the residue position corresponds to K191 and K460 of UniProtKB accession number P51784. In some cases, the modified enzyme is Ubiquitin carboxyl-terminal hydrolase 14 (USP14) and the site of modification comprises K214, wherein the residue position corresponds to K214 of UniProtKB accession number P54578. In some cases, the covalent bond is formed by reaction with a non-naturally occurring small molecule probe having a structure of Formula (I):

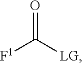

wherein F¹ is a small molecule fragment moiety comprising an alkyne moiety, a fluorophore moiety, a labeling group, or a combination thereof; and LG is a leaving group moiety. In some cases, F¹ comprises an alkyne moiety or a fluorophore moiety. In some cases, LG comprises a succinimide moiety or a phenyl moiety. In some cases, the covalent bond is formed by reaction with a non-naturally occurring ligand-electrophile having a structure of Formula (II):

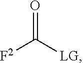

wherein F² is a small molecule fragment moiety; and LG is a leaving group moiety.

In some embodiments, one or more proteins involved in gene expression, replication, and/or nucleic acid binding are modified and the modified proteins each independently comprise a small molecule fragment moiety, covalently bonded to a lysine residue of a protein involved in gene expression, replication, and/or nucleic acid binding. In some instances, the one or more proteins comprise Histone H1.4 (HIST1H1E), Nuclear ubiquitous casein and cyclin-dependent kinase substrate 1 (NUCKS 1), Ubiquitin-40S ribosomal protein S27a (RPS27A), Paired amphipathic helix protein Sin3a (SIN3A), Transcription activator BRG1 (SMARCA4), Small ubiquitin-related modifier 1 (SUMO1), Ubiquitin-60S ribosomal protein L40 (UBA52), Ubiquitin domain-containing protein UBFD1 (UBFD1), or any combination thereof. In some cases, the modified protein is Histone H1.4 (HIST1H1E) and the site of modification comprises K90, wherein the residue position corresponds to K90 of UniProtKB accession number P10412. In some cases, the modified protein is Nuclear ubiquitous casein and cyclin-dependent kinase substrate 1 (NUCKS 1) and the site of modification comprises K175, wherein the residue position corresponds to K175 of UniProtKB accession number Q9H1E3. In some cases, the modified protein is Ubiquitin-40S ribosomal protein S27a (RPS27A) and the site of modification comprises K11, K63, K104, or K152, wherein the residue positions correspond to K11, K63, K104, and K152 of UniProtKB accession number P62979. In some cases, the modified protein is Paired amphipathic helix protein Sin3a (SIN3A) and the site of modification comprises K155 or K337, wherein the residue positions correspond to K155 and K337 of UniProtKB accession number Q96ST3. In some cases, the modified protein is Transcription activator BRG1 (SMARCA4) and the site of modification comprises K188, wherein the residue position corresponds to K188 of UniProtKB accession number P51532. In some cases, the modified protein is Small ubiquitin-related modifier 1 (SUMO1) and the site of modification comprises K37, wherein the residue position corresponds to K37 of UniProtKB accession number P63165. In some cases, the modified protein is Ubiquitin-60S ribosomal protein L40 (UBA52) and the site of modification comprises K93, wherein the residue position corresponds to K93 of UniProtKB accession number P62987. In some cases, the modified protein is Ubiquitin domain-containing protein UBFD1 (UBFD1) and the site of modification comprises K126 or K149, wherein the residue positions correspond to K126 and K149 of UniProtKB accession number O14562. In some cases, the covalent bond is formed by reaction with a non-naturally occurring small molecule probe having a structure of Formula (I):

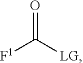

wherein F¹ is a small molecule fragment moiety comprising an alkyne moiety, a fluorophore moiety, a labeling group, or a combination thereof; and LG is a leaving group moiety. In some cases, F¹ comprises an alkyne moiety or a fluorophore moiety. In some cases, LG comprises a succinimide moiety or a phenyl moiety. In some cases, the covalent bond is formed by reaction with a non-naturally occurring ligand-electrophile having a structure of Formula (II):

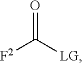

wherein F² is a small molecule fragment moiety; and LG is a leaving group moiety.

In some embodiments, one or more proteins involved in scaffolding, modulator, and/or adaptor function are modified and the modified proteins each independently comprise a small molecule fragment moiety, covalently bonded to a lysine residue of a protein involved in scaffolding, modulator, and/or adaptor function. In some instances, the one or more proteins comprise Proteasomal ubiquitin receptor ADRM1 (ADRM1), Cullin-2 (CUL2), Cullin-3 (CUL3), Cullin-4B (CUL4B), Proteasome activator complex subunit 3 (PSME3), C-Jun-amino-terminal kinase-interacting protein 4 (SPAG9), or any combinations thereof. In some cases, the modified protein is Proteasomal ubiquitin receptor ADRM1 (ADRM1) and the site of modification comprises K83 or K97, wherein the residue positions correspond to K83 and K97 of UniProtKB accession number Q16186. In some cases, the modified protein is Cullin-2 (CUL2) and the site of modification comprises K489 or K719, wherein the residue positions correspond to K489 and K719 of UniProtKB accession number Q13617. In some cases, the modified protein is Cullin-3 (CUL3) and the site of modification comprises K414 or K542, wherein the residue positions correspond to K414 and K542 of UniProtKB accession number Q13618. In some cases, the modified protein is Cullin-4B (CUL4B) and the site of modification comprises K715, wherein the residue position corresponds to K715 of UniProtKB accession number Q13620. In some cases, the modified protein is Proteasome activator complex subunit 3 (PSME3) and the site of modification comprises K14, K110, K192, K212, or K237, wherein the residue position corresponds to K14, K110, K192, K212, and K237 of UniProtKB accession number P61289. In some cases, the modified protein is C-Jun-amino-terminal kinase-interacting protein 4 (SPAG9) and the site of modification comprises K653, wherein the residue position corresponds to K653 of UniProtKB accession number O60271. In some cases, the covalent bond is formed by reaction with a non-naturally occurring small molecule probe having a structure of Formula (I):

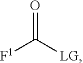

wherein F¹ is a small molecule fragment moiety comprising an alkyne moiety, a fluorophore moiety, a labeling group, or a combination thereof; and LG is a leaving group moiety. In some cases, F¹ comprises an alkyne moiety or a fluorophore moiety. In some cases, LG comprises a succinimide moiety or a phenyl moiety. In some cases, the covalent bond is formed by reaction with a non-naturally occurring ligand-electrophile having a structure of Formula (II):

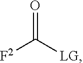

wherein F² is a small molecule fragment moiety; and LG is a leaving group moiety.

In some embodiments, one or more proteins selected from Ubiquitin-like protein ISG15 (ISG15), Small ubiquitin-related modifier 3 (SUMO3), Ubiquitin-fold modifier 1 (UFM1), or any combinations thereof, are modified and the modified proteins each independently comprise a small molecule fragment moiety, covalently bonded to a lysine residue of a protein selected from Ubiquitin-like protein ISG15 (ISG15), Small ubiquitin-related modifier 3 (SUMO3), or Ubiquitin-fold modifier 1 (UFM1). In some cases, the modified protein is Ubiquitin-like protein ISG15 (ISG15) and the site of modification comprises K35, wherein the residue position corresponds to K35 of UniProtKB accession number P05161. In some cases, the modified protein is Small ubiquitin-related modifier 3 (SUMO3) and the site of modification comprises K44, wherein the residue position corresponds to K44 of UniProtKB accession number P55854. In some cases, the modified protein is Ubiquitin-fold modifier 1 (UFM1) and the site of modification comprises K34, wherein the residue position corresponds to K34 of UniProtKB accession number P61960. In some cases, the covalent bond is formed by reaction with a non-naturally occurring small molecule probe having a structure of Formula (I):

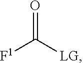

wherein F¹ is a small molecule fragment moiety comprising an alkyne moiety, a fluorophore moiety, a labeling group, or a combination thereof; and LG is a leaving group moiety. In some cases, F¹ comprises an alkyne moiety or a fluorophore moiety. In some cases, LG comprises a succinimide moiety or a phenyl moiety. In some cases, the covalent bond is formed by reaction with a non-naturally occurring ligand-electrophile having a structure of Formula (II):

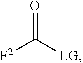

wherein F is a small molecule fragment moiety; and LG is a leaving group moiety.

Cells, Analytical Techniques, and Instrumentation

In certain embodiments, one or more of the methods disclosed herein comprise a sample (e.g., a cell sample, or a cell lysate sample). In some embodiments, the sample for use with the methods described herein is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the sample for use with the methods described herein is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the sample for use with the methods described herein is obtained from cells of a tumor cell line. In some instances, the sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the sample for use with the methods described herein is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the sample for use in the methods is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the sample is a tissue sample, such as a sample obtained from a biopsy or a tumor tissue sample. In some embodiments, the sample is a blood serum sample. In some embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

Sample Preparation and Analysis

In some embodiments, the sample (e.g., cell sample, cell lysate sample, or comprising isolated proteins) is a sample solution. In some instances, the sample solution comprises a solution such as a buffer (e.g. phosphate buffered saline) or a media. In some embodiments, the media is an isotopically labeled media. In some instances, the sample solution is a cell solution.

In some embodiments, the sample (e.g., cell sample, cell lysate sample, or comprising isolated proteins) is incubated with one or more compound probes for analysis of protein-probe interactions. In some instances, the sample (e.g., cell sample, cell lysate sample, or comprising isolated proteins) is further incubated in the presence of an additional compound probe prior to addition of the one or more probes. In other instances, the sample (e.g., cell sample, cell lysate sample, or comprising isolated proteins) is further incubated with a non-probe small molecule ligand, in which the non-probe small molecule ligand does not contain a photoreactive moiety and/or an alkyne group. In such instances, the sample is incubated with a probe and non-probe small molecule ligand for competitive protein profiling analysis.

In some cases, the sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as described in Weerapana, et al., “Quantitative reactivity profiling predicts functional cysteines in proteomes,” Nature 468(7325): 790-795.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage KIF endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electro spray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the probe-protein complex. In some embodiments, the value assigned to each of the protein from the probe-protein complex is obtained from the mass spectroscopy analysis. In some instances, the value is the area-under-the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

Kits/Article of Manufacture

Disclosed herein, in certain embodiments, are kits and articles of manufacture for use with one or more methods described herein. In some embodiments, described herein is a kit for generating a protein comprising a photoreactive ligand. In some embodiments, such kit includes photoreactive small molecule ligands described herein, small molecule fragments or libraries and/or controls, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1

Preparation of Human Cancer Cell Line Proteomes.

All cell lines were obtained from ATCC, tested negative for mycoplasma contamination, and were used without further authentication, maintaining a low passage number (<20 passages). Cell lines were grown at 37° C. with 5% CO₂. MDA-MB-231 (ATCC: HTB-26), and HEK-293T (ATCC: CRL-3216) cells were grown in DMEM medium (Corning, 15-013-CV) supplemented with 10% fetal bovine serum (FBS, Omega Scientific, FB-11, Lot #441224), penicillin, streptomycin and glutamine. Jurkat A3 (ATCC: CRL-2570) and Ramos (ATCC: CRL-1596) cells were grown in RPMI-1640 medium (Corning, 15-040-CV) supplemented with 10% FBS, penicillin, streptomycin and glutamine. For in vitro labeling, cells were grown to 100% confluence for MDA-MB-231 cells or until cell density reached 1.5 million cells per ml for Ramos and Jurkat cells. Cells were washed with cold PBS, scraped with cold PBS and cell pellets were isolated by centrifugation (1,400 g, 3 min, 4° C.), and stored at −80 OC until use. Cell pellets were resuspended in PBS, lysed by sonication and fractionated (100,000 g, 45 min) to yield soluble and membrane fractions, which were then adjusted to a final protein concentration of 1.8 mg ml⁻¹ (soluble fraction) for compound screening by competitive isoTOP-ABPP and 1.5 mg ml⁻¹ (soluble fraction) or 3 mg ml⁻¹ (membrane fraction) for reactivity measurements by isoTOP-ABPP. For gel-based ABPP lysates were adjusted to 1.8 mg ml⁻¹ (soluble fraction) for MBA-MB-231 lysates and 1 mg ml⁻¹ (soluble fraction) for HEK 293T lysates expressing target proteins. The lysates were prepared fresh from frozen pellets directly before each experiment. Protein concentration was determined using the Bio-Rad DC™ protein assay kit.

isoTOP-ABPP Sample Preparation.

In Vitro Covalent Fragment Treatment for isoTOP-ABPP.

All compounds were made up as solutions in DMSO (100×) and were used at a final concentration of 50 μM for activated esters and 100 μM for guanidinylating agents. For each profiling sample, 0.5 ml of lysate was treated with 5 μl of the 100× compound stock solution or 5 μl of DMSO. Samples were treated with activated esters for

1 h and with guanidinylating agents for 4 h.

STP-Alkyne Labeling and Click Chemistry.

For concentration-dependent reactivity measurements by isoTOP-ABPP, 0.5 ml proteome aliquots were treated at ambient temperature with 1 mM STP-alkyne 1 (5 μl of 100 mM stock in DMSO) and 0.1 mM STP alkyne 1 (5 μl of 10 mM stock in DMSO), respectively. For competitive isoTOP-ABPP, after in vitro fragment treatment (detailed above), the samples were labeled for 1 h at ambient temperature with 0.1 mM STP-alkyne 1 (5 μl of 10 mM stock in DMSO). Samples were conjugated by copper-mediated azide-alkyne cycloaddition (CuAAC) to either the light (1 mM STP-alkyne or fragment treated) or heavy (0.1 mM STP-alkyne or DMSO treated) TEV tags (10 μl of 5 mM stocks in DMSO, final concentration=100 μM) using tris(2-carboxyethyl)phosphine hydrochloride

(TCEP; fresh 50× stock in water, final concentration=1 mM), TBTA ligand (17× stock in DMSO:t-butanol 1:4, final concentration=100 μM) and CuSO₄ (50× stock in water, final concentration=1 mM). The samples were allowed to react for 1 h at room temperature, at which point the proteins from combined light and heavy samples were precipitated by chloroform-methanol extraction. The pellets were solubilized in PBS containing 1.2% SDS (1 ml) with sonication and heating (5 min, 95° C.) and any insoluble material was removed by an additional centrifugation step at ambient temperature (5,000 g, 10 min).

Streptavidin Enrichment.

For each sample, 100 μl of streptavidin-agarose beads slurry (Pierce, 20349) was washed in 10 ml PBS (3×) and then resuspended in 6 ml PBS. The SDS-solubilized proteins were added to the suspension of streptavidin-agarose beads and the bead mixture was rotated for 3 h at ambient temperature. After incubation, the beads were pelleted by centrifugation (2,800 g, 3 min) and were washed (1×10 ml 0.2% SDS in PBS, 2×10 ml PBS and 2×10 ml water).

Trypsin and TEV Digestion.

The beads were transferred to Eppendorf tubes with 1 ml PBS, centrifuged (20,000 g, 1 min), and resuspended in PBS containing 6 M urea (500 μl). To this was added 10 mM DTT (25 μl of a 200 mM stock in water) and the beads were incubated at 65° C. for 15 min. 20 mM iodoacetamide (25 μl of a 400 mM stock in water) was then added and allowed to react at 37° C. for 30 min with shaking. The bead mixture was diluted with 950 μl PBS, pelleted by centrifugation (20,000 g, 1 min), and resuspended in PBS containing 2M urea (200 μl). To this was added 1 mM CaCl₂(2 μl of a 200 mM stock in water) and trypsin (2 μg, Promega, sequencing grade in 4 μl trypsin resuspension buffer) and the samples were allowed to digest overnight at 37° C. with shaking. The beads were separated from the digest with Micro Bio-Spin columns (Bio-Rad) by centrifugation (800 g, 30 sec), washed (2×1 ml PBS and 2×1 ml water) and then transferred to fresh Eppendorf tubes with 1 ml water. The washed beads were washed once further in 140 μl TEV buffer (50 mM Tris, pH 8, 0.5 mM EDTA, 1 mM DTT) and then resuspended in 140 μl TEV buffer. 5 μl TEV protease (80 μM stock solution) was added and the reactions were rotated overnight at 30° C. The TEV digest was separated from the beads with Micro Bio-Spin columns by centrifugation (8,000 g, 3 min) and the beads were washed once with water (100 μl). The samples were then acidified to a final concentration of 5% (v/v) formic acid and stored at −80° C. prior to analysis.

Liquid-chromatography-mass-spectrometry (LC-MS) analysis of isoTOP-ABPP samples.

TEV digests were pressure loaded onto a 250 jtm (inner diameter) fused silica capillary columns packed with C18 resin (Aqua 5 m, Phenomenex). The samples were analyzed by multidimensional liquid chromatography tandem mass spectrometry (MudPIT), using an LTQ-Velos Orbitrap mass spectrometer (Thermo Scientific) coupled to an Agilent 1200-series quaternary pump. The peptides were eluted onto a biphasic column with a 5 jm tip (100 jm fused silica, packed with C18 (10 cm) and bulk strong cation exchange resin (3 cm, SCX, Phenomenex)) in a 5-step MudPIT experiment, using 0%, 30%, 60%, 90%, and 100% salt bumps of 500 mM aqueous ammonium acetate and using a gradient of 5-100% buffer B in buffer A (buffer A: 95% water, 5% acetonitrile, 0.1% formic acid; buffer B: 20% water, 80% acetonitrile, 0.1% formic acid) as has been described Weerapana, et. al., “Tandem orthogonal proteolysis-activity-based protein profiling (TOP-ABPP)—a general method for mapping sites of probe modification in proteomes. Nat. Protoc. 2, 1414-1425 (2007). Data was collected in data-dependent acquisition mode with dynamic exclusion enabled (20 s, repeat count of 2). One full MS (MS1) scan (400-1800 m/z) was followed by 30 MS2 scans (ITMS) of the nth most abundant ions.

Peptide and Protein Identification.

The MS2 spectra were extracted from the raw file using RAW Xtractor. MS2 spectra were searched using the ProLuCID algorithm using a reverse concatenated, nonredundant variant of the Human UniProt database (release-2012_11). Cysteine residues were searched with a static modification for carboxyamidomethylation (+57.02146). For all competitive and reactivity profiling experiments, lysine residues were searched with up to one differential modification for either the light or heavy TEV tags (+464.2491 or +470.26331, respectively). Peptides were required to have at least one tryptic terminus and to contain the TEV modification. ProLuCID data was filtered through DTASelect (version 2.0) to achieve a peptide false-positive rate below 1%.

Differential Labeling Analysis of Residues Labeled by Probe 1.

For analysis of the residues labeled by probe 1, peptide and protein identification was conducted as detailed above with differential modification for either the light or heavy TEV tags (+464.2491 or +470.26331, respectively) allowed on lysine, arginine, aspartate, glutamate, histidine, serine, threonine, tyrosine, asparagine, glutamine and tryptophan. Cysteine was searched with a differential modification for either the light or heavy TEV tags (+413.24185 and +407.22764, respectively).

R Value Calculation and Processing.

The ratios of light and heavy MS1 peaks for each unique peptide were quantified with a CIMAGE software using default parameters (3 MS1 acquisitions per peak and signal to noise threshold set to 2.5). For reactivity measurements by isoTOP-ABPP, the R value was calculated from the ratio of MS1 peak areas, comparing the 1 mM STP alkyne sample (light TEV tag) with the 0.1 mM STP alkyne sample (heavy TEV tag). For competitive isoTOP-ABPP, the R value was calculated from the ratio of MS1 peak areas, comparing the DMSO treated sample (heavy TEV tag) with the compound treated sample (light TEV tag). For peptides that showed a ≥95% reduction in MS1 peak area in both reactivity and compound treated samples a maximal ratio of 20 was assigned. Ratios for unique peptide entries are calculated for each experiment; overlapping peptides with the same modified lysine (for example, different charge states, MudPIT chromatographic steps or tryptic termini) are grouped together and the median ratio is reported as the final ratio (R). The peptide ratios reported by CIMAGE were further filtered to ensure the removal or correction of low-quality ratios in each individual data set. The quality filters applied were the following: removal of half tryptic peptides; for ratios with high standard deviations from the median (90% of the median or above) the lowest ratio was taken instead of the median; removal of peptides with R=20 and only a single MS2 event triggered during the elution of the parent ion; manual annotation of all the peptides with ratios of 20, removing any peptides with low quality elution profiles that remained after the previous curation steps (only done for competitive isoTOP-ABPP).

Cross-Data Processing for Fragment Screening.

For compound treated samples, biological replicates of the same condition were averaged, if the standard deviation was below 60% of the mean; otherwise, for lysines with at least one R value <4 for a particular compound, the lowest value of the ratio set was taken. For lysines, where all R values for a particular compound were ≥4, the average was reported. For peptides containing several possible modified lysines, the lysine with the highest number of quantification events was used for analysis and the remaining, redundant peptides were reported as alternative modification sites. Peptides included in the aggregate dataset (those used for further bioinformatics and statistical analyses) were required to have been quantified in 2 experiments for competitive isoTOP-ABPP. Lysines were categorized as liganded, if they had at least one ratio R≥4 (hit fragments). For liganded lysines with R=20 for all liganding events, lysines were required to have been quantified with R=20 in two separate experiments and were further required to have been quantified with R<20 in one additional experiment.

Cross-Data Processing for Reactivity Profiling.

For reactivity profiling, the median of biological replicates of the same condition and cell-line was calculated. For peptides containing several possible modified lysines, the lysine with the highest number of quantification events was used for analysis and the remaining, redundant peptides were reported as alternative modification sites. Peptides were required to be detected in at least one 1 mM vs 0.1 mM and one 0.1 mM vs 0.1 mM data set with the latter R value being smaller than 2.5. All ratios derived from soluble reactivity experiments were averaged. If the lysine was not detected in any soluble fraction, the R value from the membrane fraction was taken. Additionally, all membrane-only lysines with reactivity values were further required to have been detected in at least one 0.1 mM vs 0.1 mM membrane profiling experiment. If the final reactivity value was >10, it was set to 10. Lysines were categorized based on the R values (hyper-reactive: R<2; moderately-reactive: R=2-5; low-reactive: R>5).

Heatmap Generation.

Heat maps were generated in R (v.3.1.3) using the heatmap.2 algorithm.

Drugbank.

Proteins were queried against the DrugBank database (v. 5.0.3 released on 2016-10-24; group “All”) and separated into DrugBank and non-DrugBank proteins.

Protein Class Analysis.

To place each human protein into a distinct protein class, custom python scripts were written to parse the KEGG BRITE and Gene Ontology databases. Top level terms from KEGG were placed into a list for each protein. Enzymes were given preference for cases with multiple terms, and term-lists without enzymes were reduced by giving preference to the least frequently occurring term across the entire dataset. Gene Ontology terms and hierarchies were obtained from Superfamily, and the hierarchy tree was traversed to find more general terms for each protein. A library was constructed to place each Gene Ontology term into a category (Transporter, Channel and Receptors; Enzymes; Gene Expression and Nucleic Acid Binding; Scaffolding, Modulators and Adaptors). If a protein had Gene Ontology terms in different categories, the abovementioned order of categories was used to prioritize the protein class. If no Gene Ontology term was available that could be assigned to a category, the protein was sorted into the category “Uncategorized”. For the final protein class, the KEGG BRITE term was used, if available. If no KEGG BRITE term was available, the Gene Ontology term was used.

Functional Annotation of Lysines.

Lysines proximal to functional sites were defined as any lysine with a Cα atom within 10 Å of an annotated ligand binding site in an X-ray or NMR structure. Custom Python scripts were developed to collect relevant NMR and X-ray structures, including any co-crystallized small molecules, from the RCSB Protein Data Bank (PDB). The following small molecules were excluded from this analysis: MES, EDO, DTT, BME, ACR, ACY, ACE and MPD. Histograms of the frequency of functional sites for hyper-reactive, moderately-reactive and low reactive lysines were calculated.

Analysis of Lysine Conservation.

Sequences of all human proteins were downloaded from UniProtKB. Orthologs of human proteins were obtained using the HUGO Gene Name Consortium's database, or the DRSC Integrative Ortholog Prediction Tool, provided by Harvard Medical School. Clustal Omega was used to generate multiple sequence alignments for each human protein and its orthologs, and in-house software was used to calculate the conservation of individual lysines. Proteins with orthologues in all five organisms evaluated (M. musculus, X. laevis, D. malanogaster, C. elegans and D. rerio) were considered for the conservation analysis.

Analysis of Lysine Ubiqitylation and Acetylation.

Custom python scripts were used to compile ubiquitylation and acetylation sites and the frequency of modification at each lysine for human, mouse and rat proteomes available from the PhosphoSitePlus® (release-060616). To be considered acetylated or ubiquitylated, lysines were required to be modified with the respective PTM with a frequency of 10 or greater detection events. The percentage of total lysines modified within each reactivity range (hyper-reactive: R<2; moderately-reactive: R=2-5; low-reactive: R>5) was calculated.

Pocket Analysis.

Proteins, for which crystallographic structures were available and labeled lysines were detected, were selected for the structural analysis. UniProt accession codes were used to filter the PDB, selecting structures determined by X-ray crystallography (resolution 3.5 Å or better). Results were then filtered to select entries with the largest sequence coverage. The following proteins have been analyzed (PDB-ID in parentheses): 000299 (3o3t), 014737 (2k6b), P00367 (111f), P04179 (1pl4), P04181 (1gbn), P04632 (4phj), P07195 (1i0z), P07355 (1w7b), P07954 (3e04), P08133 (1m9i), P08237 (4omt), P08758 (2xo2), P09429 (2yrq), P11413 (1qki), P11766 (2fzw), P12268 (1nf7), P12956 (3rzx), P13804 (2alu), P15121 (4lbs), P15311 (4rm8), P18669 (1yjx), P19367 (1cza), P19784 (3e3b), P20839 (1jcn), P23284 (3ici), P23368 (1pj3), P23381 (1r6t), P23919 (1nmy), P24941 (4ek4), P26038 (1e5w), P30040 (2qc7), P36551 (2aex), P39748 (1u11), P42330 (1zq5), P49458 (4uyk), P50583 (4ick), P51580 (2bzg), P52292 (4wv6), P55145 (2w51), P55263 (4o11), P58546 (3aaa), P60520 (4co7), P61081 (1y8x), P61978 (1zzk), P62258 (3ual), P62826 (4hat), P62937 (4n1m), P68036 (4q5e), P78417 (3vln), Q01469 (5hz5), Q01813 (4xyj), Q13011 (2vre), Q13630 (4e5y), Q14914 (2y05), Q16851 (4r7p), Q5VW32 (3zxp), Q6YN16 (3kvo), Q8WUM4 (2r05), Q92600 (4cru), Q96HE7 (3ahq), Q9BSH5 (3k1z), Q9GZQ8 (5d94), Q9NTK5 (2ohf), Q9NVS9 (lnrg), Q9UBT2 (5fq2), Q9Y2Q3 (1yzx), Q9Y696 (2d2z). Structural issues (i.e., missing atoms, non-standard residues) were fixed, and wild-type amino acids restored; biological units were built using the ProDy Python module, and structures curated removing chemical entities other than standard amino acids or catalytic metals. Hydrogens were added using Reduce using default ‘build’ options. Alternate conformations were removed, then AutoDock PDBQT files were generated following the standard protocol. Pocket analysis was performed with AutoSite using neighbor_cutoff=16 for pocket clustering tolerance. For each pocket, lysines within 3.5 Å from any pocket volume points were considered adjacent.

Sequence Motifs.

For all lysines quantified in the reactivity profiling experiments, the flanking sequence (±8 amino acids) was determined with a custom python script, parsing the UniProtKB entries for all proteins identified. The sequences were binned by lysine reactivity (hyper-reactive: R<2; moderately-reactive: R=2-5; low-reactive: R>5) and evaluated for sequence motifs using WebLogo. WebLogo was created by: Gavin E. Crooks, Gary Hon, John-Marc Chandonia and Steven E. Brenner, Computational Genomics Research Group, Department of Plant and Microbial Biology, University of California, Berkeley.

Lysine Reactivity and Ligandability Comparison.

Lysines found in both the reactivity and ligandability data sets were sorted on the basis of their reactivity values (lower ratio indicates higher reactivity). The moving average of the percentage of total liganded lysines within each reactivity bin (step-size 200) was taken. See Table 4.

Subcloning and Mutagenesis.

Unless noted below, genes were amplified from cDNA prepared from low passage HEK 293T cells using the Ribozol RNA extraction reagent (Amresco) and the iScript Reverse Transcription Supermix kit (Bio-Rad). For the following proteins cDNA clones were used for amplification instead: PFKP (5180268, Dharmacon), HK1 (BC008730, transomic), SIN3A (BC137098, transomic), G6PD (BC000337, transomic) and TGIF1 (BC031268, transomic). Mouse CARM1 in pFLAG-CMV-6c was a kind gift from the Mowen lab (TSRI). NUDT2 was obtained as synthesized gene (IDT). DNA was amplified with custom forward and reverse primers using phusion polymerase (NEB, M0530S), digested with the indicated restriction enzyme and ligated into pFLAG-CMV-6c or pRK5 with the appropriate affinity tag. Lysine mutants were generated using QuikChange site-directed mutagenesis using Phusion® High-Fidelity DNA Polymerase and primers containing the desired mutations and their respective complements. The cloning of TTR and its K35A mutant has been described in Choi et al., “Chemoselective small molecules that covalently modify one lysine in a non-enzyme protein in plasma,” Nat. Chem. Biol. 6, 133-139 (2010). TTR was expressed in E. coli and purified as described. For gel-based experiments 1 μM TTR was added into 1 mg ml⁻¹ soluble MDA-MB-231 lysate.

Recombinant Expression of Proteins by Transient Transfection.

HEK 293T cells were grown to 50% confluency in 10 ml DMEM supplemented with 10% fetal bovine serum (FBS), penicillin, streptomycin and glutamine in 10 cm tissue culture dishes. 3 μg of DNA was diluted in 500 μL DMEM and 30 μL of PEI (MW 40,000, 1 mg ml⁻¹, Polysciences) were added. The mixture was incubated at room temperature for 30 min and added dropwise to the cells. Cells were grown for 48 h at 37° C. with 5% CO₂. Cells were washed with cold PBS, scraped with cold PBS and cell pellets were isolated by centrifugation (1,400 g, 3 min, 4° C.), and stored at −80° C. until use. Cell pellets were resuspended in PBS, lysed by sonication and fractionated (100,000 g, 45 min) to yield soluble and membrane fractions. The soluble fraction was adjusted to a final protein concentration of 1 mg ml⁻¹ for gel-based ABPP experiments.

Assessment of the Reactivity of Alkyne-Containing Ester Probes.

50 μL of soluble MDA-MB-231 proteome (1.8 mg ml⁻¹) were treated with 100 μM of the indicated probe (1-15) for 1 h at room temperature. Copper-mediated azide-alkyne cycloaddition (CuAAC) was performed with 25 μM rhodamine-azide (50× stock in DMSO), tris(2-carboxyethyl)phosphine hydrochloride (TCEP; fresh 50× stock in water, final concentration=1 mM), TBTA ligand (17× stock in DMSO:t-butanol 1:4, final concentration=100 rtM) and CuSO₄ (50× stock in water, final concentration=1 mM). Samples were allowed to react for 1 h at ambient temperature. The reactions were quenched with 20 μl of 4×SDS-PAGE loading buffer and the quenched samples analyzed by SDS-PAGE (10%, 14% or 16% polyacrylamide; 20 μl of sample/lane) and visualized by in-gel fluorescence using a flatbed fluorescent scanner (BioRad ChemiDoc™ MP).

Direct Labeling of Recombinantly Expressed Proteins by Gel-Based ABPP.

50 μL of soluble HEK 293T proteome (1 mg ml⁻¹) expressing the respective protein (WT or KR mutant) or transfected with an empty vector were treated with 10 μM of the indicated probe for 1 h at room temperature. The samples were analyzed as described in the previous section. For quantification of relative labeling of the different protein variants, the intensity of labeling was determined by quantifying the integrated optical intensity of the bands using ImageLab 5.2.1 software (BioRad).

Competitive Gel-Based ABPP and Apparent IC₅₀ Values.

50 μl of soluble proteome (1 mg ml⁻¹) expressing the indicated protein were treated with fragment electrophiles (1 μl of 50× stock solution in DMSO) at ambient temperature for 1 h. The indicated probe (fluorophore or alkyne-containing, 1 μl of a 500 μM solution, final concentration=10 μM) was then added and allowed to react for an additional 1 h. CuAAC and in-gel fluorescence analysis were performed as described above. For quantification of inhibition and apparent IC₅₀ determination, the percentage of labeling was determined by quantifying the integrated optical intensity of the bands using ImageLab 5.2.1 software (BioRad). Nonlinear regression analysis was used to determine the IC₅₀ values from a dose-response curve generated using GraphPad Prism 7.

PFKP Functional Assay.

For inhibitor experiments, 50 μl of soluble proteome (initial total protein concentration: 1 mg ml⁻¹) from HEK 293T cells expressing PFKP (WT or K688R mutant) or mock transfected cells (empty vector; negative control) were incubated with 1 μl 50× of the compound in DMSO or DMSO for the positive or negative control for 1 h at room temperature. Lysates were diluted 40× with dilution buffer (PBS containing 0.2 mg ml⁻¹ BSA and 5 mM MgCl₂) and 40 μl were added into a clear bottom 384 well plate. 10 μl of a mixture of 3.5 μl PBS, 2.5 μl fructose-6-phosphate (100 mM), 1 μl NADH (20 mM), 1 μl ATP (50 mM), 1 μl aldolase (50 U ml⁻¹) and 1 μl GDH/TPI (500 U ml⁻¹ TPI, 50 U ml⁻¹ GDH) were added to start the reaction. The absorbance of NADH was measured at 340 nm every minute for 30 min.

PNPO Functional Assay.

80 μl of soluble proteome (total protein concentration: 1 mg ml⁻¹) from HEK 293T cells expressing PNPO (WT or K100R mutant) or mock transfected cells (empty vector; negative control) were added into a clear bottom 384 well plate. For compound treatments, 1 μl of the inhibitor (80× solution in DMSO) or 1 μl of DMSO (positive control) were added and the reactions were incubated for 1 h at room temperature. 10 μl of 0.1 M Tris in PBS were added and the reaction was started by addition of 10 μl 5 mM pyridoxine phosphate (PNP) in water (PNP was prepared as described in Argoudelis, C. J., “Preparation of crystalline pyridoxine 5′-phosphate and some of its properties,” J. Agr. Food Chem. 34, 995-998 (1986)). The absorbance of the Schiff Base between pyridoxal phosphate and Tris was measured at 388 nm every minute for 30 min.

G6PD Functional Assay.

Soluble proteome (initial total protein concentration: 1 mg ml⁻¹) from HEK 293T cells expressing G6PD (WT or K171R mutant) or mock transfected cells (empty vector; negative control) were diluted 1000× with dilution buffer. 88 μl of this were added into a clear bottom 384 well plate. 12 μl of a mixture of 8 μl water, 2 μl 60 mM glucose-6-phosphate and 2 μl 20 mM NADP were added to start the reaction. The absorbance of NADPH was measured at 340 nm every minute for 30 min.

NUDT2 Functional Assay.

NUDT2 activity was measured with a published assay using a fluorogenic substrate. For inhibitor experiments, 50 μl of soluble proteome (initial total protein concentration: 1 mg ml⁻¹) from HEK 293T cells expressing NUDT2 (WT or K89R mutant) or mock transfected cells (empty vector; negative control) were incubated with 1 μl 50× of the compound in DMSO or DMSO for the positive or negative control (lysate transfected with empty vector) for 1 h at room temperature. Lysates were diluted 4000× with dilution buffer and 64 μl were added into a black 384 well plate. 16 μl of fluorogenic substrate (5 μM) were added to start the reaction. The fluorescence intensity with excitation at 530 nm and emission at 563 nm was measured every minute for 30 min.

Calculation of Relative Activity or Percent Inhibition.

For PNPO, PFKP, NUDT2 and G6PD, the slope of the linear regression of the linear portion of the absorbance or fluorescence over time was used as measure their activity. Apparent activity was calculated relative to the WT. Percent inhibition was calculated relative to the positive and negative control and used to calculate IC₅₀ values by nonlinear regression analysis from a dose-response curve generated using GraphPad Prism 7.

Site of labeling of recombinantely expressed proteins by reductive dimethylation (ReDiMe).

500 μl of soluble proteome from HEK 293T cells expressing the indicated proteins (1 mg ml⁻¹ total protein concentration; see recombinant expression of proteins by transient transfection for additional details) were treated with the indicated compound at 50 μM (5 μl of 5 mM stock in DMSO) or DMSO for 1 h at ambient temperature. For each sample, 20 μl anti-FLAG® M1 Agarose Affinity Gel (Sigma, A4596) slurry was washed once by centrifugation with 500 μl 0.1 M glycine pH 3.5 and three times with 500 μl PBS (8,000 g, 3 min). The compound- and DMSO-treated reactions were separately enriched on anti-FLAG resin for 4 h at 4° C. while rotating. The beads were collected by centrifugation (8,000 g, 3 min) and washed three times with PBS. The beads were resuspended in 80 μl 6 M Urea in TEAB (pH 8.0, 100 mM) and rotated at room temperature for 30 min to elute the captured proteins. After separation of the beads, 10 mM DTT (4 μl of 200 mM) were added and the reaction was incubated at 65° C. for 15 minutes following which 20 mM iodoacetamide (4 μl of 400 mM) was added and the reaction incubated for 30 minutes at 37° C. The samples were then diluted with TEAB (232 μl) and to this was added the appropriate restriction enzyme (trypsin (10 μl, 5 μg total) for HDHD3, HK1, SIN3A and XRCC6 or rLysC (10 μl, 5 μg total, Promega, V1671) for PNPO and PFKP) and the samples were allowed to digest over night at 37° C. with shaking. Reductive dimethylation was performed as described in Inloes, et al., “he hereditary spastic paraplegia-related enzyme DDHD2 is a principal brain triglyceride lipase,” Proc. Natl. Acad. Sci. USA 111, 14924-14929 (2014). Briefly, DMSO-treated samples were labeled with heavy-formaldehyde (¹³C,D₂-) and compound-treated samples with light formaldehyde (¹²C,H₂) (0.15% formaldehyde) and sodium cyanoborohydride (22.2 mM). After 1 h at ambient temperature with shaking, the reactions were quenched by addition of NH₄OH (2.3%) for 10 min followed by acidification with formic acid (5%). The samples were then combined and analyzed by LC/MS analysis. The MS2 spectra data were extracted from the raw file using RAW Xtractor (version 1.9.9.2). MS2 spectra data were searched using the ProLuCID algorithm using a reverse concatenated, nonredundant variant of the Human UniProt database (release-2012_11). Cysteine residues were searched with a static modification for carboxyamidomethylation (+57.02146 C). Searches also included methionine oxidation as a differential modification (+15.9949 M). Peptides were searched with a static modification for dimethylation of lysine residues (+28.0313 K) and the N-terminus (+28.0313 N-term) and for ReDiMe labeled amino acids (+6.03181 K, +6.03181 N-term). Peptides were also searched with a differential modification on lysine to detect the directly labeled peptide-compound adducts (+246.07931 for 19, +194.05791 for 33, +166.04186 for 20, +211.96968 for 21 and +143.03711 for 32). Peptides were required to have at least one cognate proteolytic terminus and unlimited missed cleavage sites. ProLuCID data was filtered through DTASelect (version 2.0) to achieve a peptide false-positive rate below 1%. Ratios of heavy/light (DMSO/test compound) peaks were calculated using a CIMAGE software. Unmodified peptides were included in the final analysis, if they stemmed from the expressed protein, contained cognate cleavage sites on both ends, contained no internal missed cleavage sites and had at least one lysine as the cleavage site.

ABPP-SILAC IP Experiment for SIN3A Interacting Proteins.

All SILAC experiments were performed using the isotopically labeled human HEK 293T cell line generated by 8 passages in either light (100 μg ml⁻¹ each of L-arginine and L-lysine) or heavy (100 μg ml⁻¹ each of [¹³C₆¹⁵N₄]L-arginine and [¹³C₆¹⁵N₂]L-lysine) SILAC DMEM media (Thermo Scientific) supplemented with 10% dialyzed fetal calf serum, penicillin, streptomycin and glutamine. 2×10⁻⁵ SILAC HEK 293T cells were plated in 6 cm dishes in either heavy or light labeled SILAC media. Cells were transfected the next day with 1 ag of FLAG-GFP, or FLAG-SIN3A wild type, K155R, or K155W constructs as indicated. After 48 hours, cells were rinsed with ice-cold PBS and suspended in cold IP-lysis buffer (0.5% Chaps, 50 mM Hepes pH 7.4, 150 mM NaCl, and EDTA-free protease inhibitors and phosphatase inhibitors (Roche)) by gentle sonication. Samples were rotated for 30 minutes at 4° C. to complete lysis. For compound treatment experiments, 50 μM (final concentration) of 21 was added to samples prior to rotation. Samples were clarified by centrifugation for 1 minute at 16,000 rpm, and protein concentration was measured using the DC Protein Assay kit (Bio-Rad). Samples were normalized to 2 mg/mL by addition of cold IP-lysis buffer. 25 μL of anti-FLAG-M2 beads was added to the clarified supernatant and incubated for 3 h while rotating at 4° C. Beads were washed three times with cold PBS, and then eluted with 40 μL of 8 M urea for 10 min at 65° C. Samples were combined and then reduced by addition of 12.5 mM DTT at 65° C. for 15 minutes. Samples were alkylated with 25 mM iodoacetamide at 37° C. for 15 minutes, then diluted to 2 M urea with PBS. Sequence grade trypsin (Promega) was reconstituted in trypsin buffer with CaCl₂, as detailed above, and 2 ag of trypsin was added to each samples. Samples were shaken at 37° C. overnight after which digests were acidified with formic acid to a final concentration of 5% (v/v). Samples were stored at −80° C. until analysis by LC-MS. LC-MS spectra were collected and analyzed as described above with the following modifications. Cysteine residues were searched with a static modification for carboxyamidomethylation (+57.02146 C). Searches also included methionine oxidation as a differential modification (+15.9949 M) and mass shifts of SILAC labeled amino acids (+10.0083 R, +8.0142 K) and no enzyme specificity. Peptides were required to have at least one tryptic terminus and unlimited missed cleavage sites. 2 peptide identifications were required for each protein. R values for co-immunoprecipitation are presented as the median ratio of heavy/light peptides for all biological replicates. A list of all proteins enriched preferentially by SIN3A was generated from a comparison of SIN3A wild type vs GFP immunoprecipitations, including all proteins with at least two distinct quantified peptide sequences and a median ratio greater than or equal to 5 (R≥5). For the wild type vs mutant or compound treatment experiments, proteins were considered for analysis, if they had been preferentially enriched in the SIN3A vs GFP experiments (R≥5). Furthermore, if there were at least two quantified unique peptides, the median ratio of each protein's unique peptides (not occurring in any other human protein) were reported.

Co-IP Experiment for the Interaction Between SIN3A and TGIF1 and TGIF2.

6 cm dishes of HEK 293T cells were transfected at 40% confluency with 600 ng of FLAG-GFP, FLAG-SIN3A WT, K155W, or K155R construct, and 600 ng of MYC-TGIF1 or MYC-TGIF2 as indicated. After 48 hours, cells were lysed and enriched as described above. Following elution in 40 μL urea, 15 μL of loading buffer was added to samples. 15 μL of both input (10%) and outputs were loaded onto an SDS-PAGE gel.

Western Blotting.

Proteins were resolved by SDS-PAGE (3 h, 300 V) and transferred to nitrocellulose membranes (90 min, 60 V), blocked with 5% milk in TBS-T and probed with the indicated antibodies in 5% milk in TBS-T. The primary antibodies and the dilutions used are as follows: anti-Flag (Sigma Aldrich, F1804, 1:3,000), anti-Myc (Cell Signalling, 2272S, 1:5,000), anti-actin (Cell Signaling, 3700, 1:3,000) and anti-GAPDH (Santa Cruz, 32233, 1:10,000). Blots were incubated with primary antibodies overnight at 4° C. with rocking and were then washed (3×5 min, TBS-T) and incubated with secondary antibodies (LICOR, IRDye 800CW or IRDye 680LT, 1:10,000) for 1 h at ambient temperature. Blots were further washed (3×5 min, TBST) and visualized on a LICOR Odyssey Scanner. Relative band intensities were quantified using ImageJ software.

Statistical Analysis.

The experiments were not randomized. The investigators were not blinded to allocation during experiments and outcome assessment. No statistical methods were used to predetermine sample size. Data are shown as mean±standard deviation of at least two experiments. Statistical significance was calculated with unpaired students t-tests; *, p<0.05, **, p<0.01, ***, p<0.001, ****, p<0.0001.

Synthetic Methods

Chemicals and reagents were purchased from a variety of vendors, including Sigma Aldrich, Acros, Fisher, Fluka, Santa Cruz, CombiBlocks, BioBlocks, and Matrix Scientific, and were used without further purification, unless noted otherwise. Anhydrous solvents were obtained as commercially available pre-dried, oxygen-free formulations. Flash chromatography was carried out using 230-400 mesh silica gel. Preparative thin layer chromotography (PTLC) was carried out using glass backed PTLC plates 500-2000 μm thickness (Analtech). All reactions were monitored by thin layer chromatography carried out on 0.25 mm E. Merck silica gel plates (60F-254) and visualized with UV light, or by ninhydrin, ethanolic phosphomolybdic acid, iodine, p-anisaldehyde or potassium permanganate stain. NMR spectra were recorded on Varian INOVA-400, Bruker DRX-600 or Bruker DRX-500 spectrometers in the indicated solvent. Multiplicities are reported with the following abbreviations: s singlet; d doublet; t triplet; q quartet; p pentet; m multiplet; br broad. Chemical shifts are reported in ppm relative to the residual solvent peak and J values are reported in Hz. Mass spectrometry data were collected on an Agilent ESI-TOF instrument (HRMS-ESI) or an Agilent 6520 Accurate-Mass Q-TOF (HRMS).

The following molecules were purchased from commercial vendors: 1 (Lumiprobe, 40720), 16 (ThermoFisher Scientific, 46410), 17 (ThermoFisher Scientific, A37570), 18 (ThermoFisher Scientific, B10006), 50 (Sigma-Aldrich, 439428) and 51 (Sigma-Aldrich, 559997).

General Procedure A.

1.23 mmol of the carboxylic acid (1.5 eq.) and 0.82 mmol of the phenol (1.0 eq) or N-hydroxysuccinimide were dissolved in 5 ml DCM and 340 μl triethylamine (247 mg, 2.44 mmol, 3.0 eq.) were added. 418 mg 2-chloro-1-methylpyridinium iodide (1.64 mmol, 2.0 eq.) were added. The mixture was stirred over night at room temperature and directly loaded onto a preparative TLC. The TLC was run with the indicated solvent and the product was eluted from the silica. Evaporation of the solvent resulted in the desired ester.

General Procedure B.

0.82 mmol of the phenol or N-hydroxysuccinimide (1.0 eq.) were dissolved in 5 ml DCM and 340 μl triethylamine (247 mg, 2.44 mmol, 3.0 eq.) were added. To this 1.23 mmol of the carbonyl chloride were added and the mixture was stirred for 4 h at room temperature. The reaction was directly loaded onto a preparative TLC. The TLC was run with the indicated solvent and the product was eluted from the silica. Evaporation of the solvent resulted in the desired ester.

### 4-Nitrophenyl 4-pentynoate (2)

This compound was synthesized according to General Procedure A starting from 4-pentynoic acid and 4-nitrophenol. The preparative TLC was run with n-hexane/DCM 1:3. 70 mg (39%) of the product were obtained. ¹H-NMR (400 MHz, CDCl₃): δ 8.28 (d, J=8.7 Hz, 2H), 7.30 (d, J=8.7 Hz, 2H), 2.86 (t, J=7.3 Hz, 2H), 2.64 (t, J=&.3 Hz, 2H), 2.07-2.04 (m, 1H); HRMS (m/z) calculated for C₁₁H₁₀NO₄ [M+H]: 220.0604; found: 220.0602.

### 2-Nitrophenyl 4-pentynoate (3)

This compound was synthesized according to General Procedure A starting from 4-pentynoic acid and 2-nitrophenol. The preparative TLC was run with n-hexane/DCM 1:3. 97 mg (54%) of the product were obtained. ¹H-NMR (400 MHz, CDCl₃): δ 8.12 (d, J=8.3 Hz, 1H), 7.67 (t, J=7.9 Hz, 1H), 7.42 (t, J=8.0 Hz, 1H), 7.27 (d, J=5.5 Hz, 1H), 2.92 (t, J=7.3 Hz, 2H), 2.66 (d, J=7.3 Hz, 2H, 2.08-2.03 (m, 1H); HRMS (m/z) calculated for C₁₁H₉NNaO₄ [M+Na]: 242.0424; found: 242.0424.

### 2,4-Dinitrophenyl 4-pentynoate (4)

This compound was synthesized according to General Procedure A starting from 4-pentynoic acid and 2,4-dinitrophenol. The preparative TLC was run with n-hexane/DCM 1:3. 192 mg (89%) of the product were obtained. ¹H-NMR (400 MHz, CDCl₃): δ 8.98 (d, J=2.6 Hz, 1H), 8.53 (dd, J=2.6 Hz, J=8.9 Hz, 1H), 7.51 (d, J=8.9 Hz, 1H), 2.96 (t, J=7.3 Hz, 2H), 2.67 (dt, J=2.6 Hz, J=7.3 Hz, 2H), 2.07 (t, J=2.6 Hz, 1H); HRMS (m/z) calculated for C₁₁H₉N₂O₆[M+H]: 265.0455; found: 265.0453.

### 2,3,5,6-Tetrafluorophenyl 4-pentynoate (5)

This compound was synthesized according to General Procedure A starting from 4-pentynoic acid and 2,3,5,6-tetrafluorophenol. The preparative TLC was run with n-hexane/DCM 1:1. 185 mg (92%) of the product were obtained. ¹H-NMR (400 MHz, CDCl₃): δ 7.06-6.95 (m, 1H), 2.94 (t, J=7.3 Hz, 2H), 2.66 (d, J=7.3 Hz, 2H), 2.07-2.04 (m, 1H); ¹⁹F-NMR (376 MHz, CDCl₃) δ −139.20 (dd, J=12.3 Hz, J=9.6 Hz, 2F), −153.07 (dd, J=12.3 Hz, J=9.6 Hz, 2F); HRMS (m/z) calculated for C₁₁H₇F₄O₂ [M+H]: 247.0377; found: 247.0380.

### Pentafluorophenyl 4-pentynoate (6)

This compound was synthesized according to General Procedure A starting from 4-pentynoic acid and pentafluorophenol. The preparative TLC was run with n-hexane/DCM 1:1. 140 mg (65%) of the product were obtained. ¹H-NMR (400 MHz, CDCl₃): δ 2.93 (t, J=7.3 Hz, 2H), 2.69-2.59 (m, 2H), 2.09-2.03 (m, 1H); ¹⁹F-NMR (376 MHz, CDCl₃) δ −152.72-−152.85 (m, 2F), −158.02 (t, J=21.7 Hz, 1F), −162.39-−162.60 (m, 2F); HRMS (m/z) calculated for C₁₁H₆F₅O₂[M+H]: 265.0283; found: 265.0280.

### 4-Trifluoromethyl-2,3,5,6-tetrafluorophenyl 4-pentynoate (7)

This compound was synthesized according to General Procedure A starting from 4-pentynoic acid and 4-trifluoromethyl-2,3,5,6-tetrafluorophenol. The preparative TLC was run with n-hexane/DCM 2:1. 168 mg (65%) of the product were obtained. ¹H-NMR (400 MHz, CDCl₃): δ 2.96 (t, J=7.2 Hz, 2H), 2.66 (d, J=7.2 Hz, 2H), 2.08-2.04 (m, 1H); ¹⁹F-NMR (376 MHz, CDCl₃) δ −56.4 (t, J=26.8 Hz, 3F), −140.43-−140.76 (m, 2F), −150.35-−150.50 (m, 2F); HRMS (m/z) calculated for C₁₂H₆F₇O₂[M+H]: 315.0251; found: 315.0252.

### 4-Pentynoic acid NHS ester (8)

This compound was synthesized according to General Procedure A starting from 4-pentynoic acid and N-hydroxysuccinimide. The preparative TLC was run with DCM/ethyl acetate 4:1. 93 mg (58%) of the product were obtained. ¹H-NMR (400 MHz, CDCl₃): δ 2.88 (t, J+2.88 Hz, 2H), 2.84 (s, 4H), 2.65-2.58 (m, 2H), 2.07-2.03 (m, 1H); HRMS (m/z) calculated for C₉H₁₀NO₄ [M+H]: 196.0604; found: 196.0598.

### 4-Nitrophenyl 4-ethynylbenzoate (9)

This compound was synthesized according to General Procedure A starting from 4-ethynylbenzoic acid and 4-nitrophenol. The preparative TLC was run with n-hexane/DCM 1:3. 74 mg (34%) of the product were obtained. ¹H-NMR (400 MHz, CDCl₃): δ 8.36-8.31 (m, 2H), 8.18-8.14 (m, 2H), 7.67-7.62 (m, 2H), 7.45-7.40 (m, 2H), 3.31 (s, 1H). ¹³C-NMR (100 MHz, CDCl₃): δ 163.73, 155.68, 145.66, 132.58, 130.33, 128.59, 128.34, 125.47, 122.72, 82.61, 81.27; HRMS (m/z) calculated for C₁₅H₁₀NO₄ [M+H]: 268.0604; found: 268.0605.

### 2-Nitrophenyl 4-ethynylbenzoate (10)

This compound was synthesized according to General Procedure A starting from 4-ethynylbenzoic acid and 2-nitrophenol. The preparative TLC was run with n-hexane/DCM 1:3. 53 mg (24%) of the product were obtained. ¹H-NMR (400 MHz, CDCl₃): δ 8.20-8.09 (m, 3H), 7.71 (dt, J=7.8, 1.2 Hz, 1H), 7.66-7.61 (m, 2H), 7.48-7.42 (m, 1H), 7.39 (dd, J=8.2, 1.2 Hz, 1H), 3.30 (s, 1H); HRMS (m/z) calculated for C₁₅H₁₀NO₄ [M+H]: 268.0604; found: 268.0602.

### 2,4-Dinitrophenyl 4-ethynylbenzoate (11)

This compound was synthesized according to General Procedure A starting from 4-ethynylbenzoic acid and 2,4-dinitrophenol. The preparative TLC was run with n-hexane/DCM 1:3. 151 mg (55%) of the product were obtained. ¹H-NMR (400 MHz, CDCl₃): δ 9.02 (s, 1H), 8.58 (d, J=9.0 Hz, 1H), 8.15 (d, J=8.1 Hz, 2H), 7.69-7.62 (m, 3H), 3.33 (s, 1H); HRMS (m/z) calculated for C₁₅H₉N₂O₆[M+H]: 313.0455; found: 313.0446.

### 2,3,5,6-Tetrafluorophenyl 4-ethynylbenzoate (12)

This compound was synthesized according to General Procedure A starting from 4-ethynyl benzoic acid and 2,3,5,6-tetrafluorophenol. The preparative TLC was run with n-hexane/DCM 2:1. 158 mg (66%) of the product were obtained. ¹H-NMR (400 MHz, CDCl₃): δ 8.19-8.15 (m, 2H), 7.67-7.62 (m, 2H), 7.06 (tt, J=9.9 Hz, J=7.1 Hz, 1H), 3.32 (s, 1H); ¹⁹F-NMR (376 MHz, CDCl₃) δ −139.03-−139.16 (m, 2F), −152.88-−153.01 (m, 2F); ¹³C-NMR (100 MHz, CDCl₃): δ 162.09, 146.24 (d, J=248.7 Hz), 140.86 (d, J=251.5 Hz), 132.61, 130.68, 129.93, 128.74, 127.19, 103.55 (t, J=21.8 Hz), 82.54, 81.46; HRMS (m/z) calculated for C₁₅H₇F₄O₂[M+H]: 295.0377; found: 295.0374.

### Pentafluorophenyl 4-ethynylbenzoate (13)

This compound was synthesized according to General Procedure A starting from 4-ethynylbenzoic acid and pentafluorophenol. The preparative TLC was run with n-hexane/DCM 2:1. 214 mg (84%) of the product were obtained. ¹H-NMR (400 MHz, CDCl₃): δ 8.16 (d, J=8.2 Hz, 2H), 7.65 (d, J=8.1 Hz, 2H), 3.33 (s, 1H); ¹⁹F-NMR (376 MHz, CDCl₃) δ −152.61-−152.73 (m, 2F), −157.90 (t, J=21.8 Hz, 1F), −162.30-−162.52 (m, 2F); HRMS (m/z) calculated for C₁₅H₆F₅O₂[M+H]: 313.0283; found: 313.0279.

### 4-Trifluoromethyl-2,3,5,6-tetrafluorophenyl 4-ethynylbenzoate (14)

This compound was synthesized according to General Procedure A starting from 4-ethynylbenzoic acid and 4-trifluoromethyl-2,3,5,6-tetrafluorophenol. The preparative TLC was run with n-hexane/DCM 2:1. 148 mg (50%) of the product were obtained. ¹H-NMR (400 MHz, CDCl₃): δ 8.16 (d, J=8.5 Hz, 2H), 7.66 (d, 8.5 Hz, 2H), 3.34 (s, 1H); ¹⁹F-NMR (376 MHz, CDCl₃) δ −56.32 (t, J=22.0 Hz, 3F), −140.35-−140.67 (m, 2F), −150.23-−150.38 (m, 2F); HRMS (m/z) calculated for C₁₆H₆F₇O₂[M+H]: 363.0251; found: 363.0252.

### 4-Ethynylbenzoic acid NHS ester (15)

This compound was synthesized according to General Procedure A starting from 4-ethynylbenzoic acid and N-hydroxysuccinimide. The preparative TLC was run with DCM/ethyl acetate 4:1. 94 mg (47%) of the product were obtained. ¹H-NMR (400 MHz, CDCl₃): δ 8.09 (d, J=8.1 Hz, 2H), 7.61 (d, J=8.1 Hz, 2H), 3.32 (s, 1H), 2.92 (s, 4H); HRMS (m/z) calculated for C₁₃H₁₀NO₄ [M+H]: 244.0604; found: 244.0598.

### Pentafluorophenyl 3-(1,3-diphenyl-1H-pyrazol-4-yl)propanoate (19)

This compound was synthesized according to General Procedure A starting from 3-(1,3-diphenyl-1H-pyrazol-4-yl)propanoic acid and pentafluorophenol. The preparative TLC was run with n-hexane/DCM 1:1. 358 mg (95%) of the product were obtained. ¹H-NMR (400 MHz, CDCl₃): δ 7.88 (s, 1H), 7.77-7.71 (m, 4H), 7.51-7.43 (m, 4H), 7.43-7.37 (m, 1H), 7.32-7.27 (m, 1H), 3.20 (t, J=7.4 Hz, 2H), 2.99 (t, J=7.4 Hz, 2H); ¹⁹F-NMR (376 MHz, CDCl₃) δ −152.86-−153.01 (m, 2F), −158.08 (t, J=21.7 Hz, 1F), −162.31-−162.54 (m, 2F); ¹³C-NMR (100 MHz, CDCl₃): δ 168.90, 151.58, 141.23 (d, J=249.2 Hz), 140.09, 139.62 (d, 237.6 Hz), 138.00 (d, J=250.8 Hz), 133.47, 129.55, 128.81, 128.18, 127.99, 126.58, 126.46, 125.08, 118.96, 118.74, 34.03, 20.01; HRMS-ESI (m/z) calculated for C₂₄H₁₆F₅N₂O₂[M+H]: 459.1126; found: 459.1126.

### Pentafluorophenyl 2,2-diphenylacetate (20)

This compound was synthesized according to General Procedure B starting from 2,2-diphenylacetyl chloride and pentafluorophenol. The preparative TLC was run with n-hexane/DCM 2:1. 274 mg (88%) of the product were obtained. ¹H-NMR (400 MHz, CDCl₃): δ 7.42-7.30 (m, 10H), 5.39 (s, 1H); ¹⁹F-NMR (376 MHz, CDCl₃) δ −152.40-−152.53 (m, 2F), −157.92 (t, J=21.7 Hz, 1F), −162.37-−162.67 (m, 2F); ¹³C-NMR (100 MHz, CDCl₃): δ 168.83, 141.30 (d, 250.5 Hz), 139.7 (d, 246.9 Hz), 137.96 (d, 262.6 Hz), 137.09, 129.05, 128.71, 128.04, 125.22, 56.49; HRMS (m/z) calculated for C₂₀H₁₂F₅O₂ [M+H]: 379.0752; found: 379.0737.

### Pentafluorophenyl 3,5-bis(trifluoromethyl)benzoate (21)

This compound was synthesized according to General Procedure B starting from 3,5-bis(trifluoromethyl)benzoyl chloride and pentafluorophenol. The preparative TLC was run with n-hexane/DCM 2:1. 244 mg (70%) of the product were obtained. ¹H-NMR (400 MHz, CDCl₃): δ 8.65 (s, 2H), 8.22 (s, 1H); ¹⁹F-NMR (376 MHz, CDCl₃) δ −63.33 (s, 6F), −152.41-−152.53 (m, 2F), −156.57 (t, J=21.7 Hz, 1F), −161.53-−161.71 (m, 2F); ¹³C-NMR (100 MHz, CDCl₃): δ 160.40, 141.33 (d, 252.8 Hz), 140.22 (d, 256.3 Hz), 137.70 (d, J=252.8 Hz), 133.13 (q, J=34.8 Hz), 130.84, 129.39, 128.22, 124.79, 122.74 (q, J=273.0 Hz); HRMS (m/z) calculated for C₁₅H₄F₁₁O₂[M+H]: 425.0030; found: 425.0036.

### Pentafluorophenyl 2-(1-methyl-1H-indol-3-yl)acetate (22)

This compound was synthesized according to General Procedure A starting from 2-(1-methyl-1H-indol-3-yl)acetic acid and pentafluorophenol. The preparative TLC was run with n-hexane/DCM 2:1. 279 mg (96%) of the product were obtained. ¹H-NMR (400 MHz, CDCl₃): δ 7.62 (d, J=7.9 Hz, 1H), 7.34 (d, J=8.2 Hz, 1H), 7.31-7.24 (m, 1H), 7.17 (t, J=7.4 Hz, 1H), 7.12 (s, 1H), 4.12 (s, 2H), 3.80 (s, 3H); ¹⁹F-NMR (376 MHz, CDCl₃) δ −152.68-−152.80 (m, 2F), −158.39 (t, J=21.7 Hz, 1F), −162.58-−162.81 (m, 2F); ¹³C-NMR (100 MHz, CDCl₃): δ 168.04, 141.27 (d, J=255.0 Hz), 139.60 (d, J=241.9 Hz), 137.94 (d, J=255.0 Hz), 137.07, 128.13, 127.50, 125.39, 122.21, 119.65, 118.72, 109.58, 104.91, 32.88, 30.35; HRMS-ESI (m/z) calculated for C₁₇H₁₁F₅NO₂ [M+H]: 356.0704; found: 356.0710.

### Pentafluorophenyl 3-(3,4,5-trimethoxyphenyl)propanoate (23)

This compound was synthesized according to General Procedure A starting from 3-(3,4,5-trimethoxyphenyl)propanoic acid and pentafluorophenol. The preparative TLC was run with DCM. 284 mg (85%) of the product were obtained. ¹H-NMR (400 MHz, CDCl₃): δ 6.46 (s, 2H), 3.86 (s, 6H), 3.83 (s, 3H), 3.08-2.95 (m, 4H); ¹⁹F-NMR (376 MHz, CDCl₃) δ −152.87-−153.09 (m, 2F), −158.12 (t, J=21.7 Hz, 1F), −162.38-−162.59 (m, 2F); ¹³C-NMR (100 MHz, CDCl₃): δ 168.86, 153.51, 141.24 (d, J=246.7 Hz), 139.61 (d, J=239.1 Hz), 137.99 (d, J=248.4 Hz), 136.90, 135.20, 125.13, 105.33, 60.98, 56.21, 35.24, 31.17; HRMS-ESI (m/z) calculated for C₁₈H₁₆F₅O₅ [M+H]: 407.0912; found: 407.0914.

### 1-Benzyl 4-(pentafluorophenyl) piperidine-1,4-dicarboxylate (24)

This compound was synthesized according to General Procedure A starting from 1-((benzyloxy)carbonyl)piperidine-4-carboxylic acid and pentafluorophenol. The preparative TLC was run with DCM. 304 mg (86%) of the product were obtained. ¹H-NMR (400 MHz, CDCl₃): δ 7.41-7.29 (m, 5H), 5.14 (s, 2H), 4.13 (s, 2H), 3.07 (t, J=11.8 Hz, 2H), 2.89 (dd, J=10.2, 3.8 Hz, 1H), 2.17-1.98 (m, 2H), 1.93-1.75 (m, 2H); ¹⁹F-NMR (376 MHz, CDCl₃) δ −153.33-−153.49 (m, 2F), −157.99 (t, J=21.7 Hz, 1F), −162.28-−162.50 (m, 2F); HRMS-ESI (m/z) calculated for C₂₀H₁₇F₅NO₄ [M+H]: 430.1072; found: 430.1071.

### Pentafluorophenyl quinoline-2-carboxylate (25)

This compound was synthesized according to General Procedure A starting from quinoline-2-carboxylic acid and pentafluorophenol. The preparative TLC was run with n-hexane/DCM 1:1. 230 mg (83%) of the product were obtained. ¹H-NMR (400 MHz, CDCl₃): δ 8.42 (d, J=8.5 Hz, 1H), 8.37 (d, J=8.6 Hz, 1H), 8.31 (d, J=8.6 Hz, 1H), 7.96 (d, J=8.2 Hz, 1H), 7.87 (t, J=7.8 Hz, 1H), 7.74 (t, J=7.6 Hz, 1H); ¹⁹F-NMR (376 MHz, CDCl₃) δ −151.99-−152.13 (m, 2F), −157.62 (t, J=21.7 Hz, 1F), −162.18-−162.38 (m, 2F); ¹³C-NMR (100 MHz, CDCl₃): δ 161.73, 147.94, 145.09, 141.45 (d, J=249.6), 139.78 (d, J=251.1 Hz), 138.12 (d, J=249.6 Hz), 137.88, 131.01 (two overlapping signals), 129.95, 129.73, 127.81, 125.66, 121.75; HRMS-ESI (m/z) calculated for C₁₆H₇F₅NO₂ [M+H]: 340.0391; found: 340.0389.

### Pentafluorophenyl 3-(7-fluoro-4-oxo-4H-chromen-3-yl)propanoate (26)

This compound was synthesized according to General Procedure A starting from 3-(7-fluoro-4-oxo-4H-chromen-3-yl)propanoic acid and pentafluorophenol. The preparative TLC was run with DCM. 307 mg (93%) of the product were obtained. ¹H-NMR (400 MHz, CDCl₃): δ 7.93 (s, 1H), 7.86 (dd, J=8.2, 2.7 Hz, 1H), 7.48 (dd, J=9.3, 4.2 Hz, 1H), 7.44-7.37 (m, 1H), 3.08 (t, J=6.9 Hz, 2H), 2.90 (t, J=6.9 Hz, 2H); ¹⁹F-NMR (376 MHz, CDCl₃) δ −115.29 (s, 1F), −152.79-−152.91 (m, 2F), −158.13 (t, J=21.7 Hz, 1F), −162.38-−162.58 (m, 2F); HRMS-ESI (m/z) calculated for C₁₈H₉F₆O₄[M+H]: 403.0400; found: 403.0400.

### Pentafluorophenyl 2-(1,3-dioxoisoindolin-2-yl)acetate (27)

This compound was synthesized according to General Procedure A starting from 2-(1,3-dioxoisoindolin-2-yl)acetic acid and pentafluorophenol. The preparative TLC was run with DCM. 257 mg (84%) of the product were obtained. ¹H-NMR (400 MHz, CDCl₃): δ 7.96-7.90 (m, 2H), 7.82-7.75 (m, 2H), 4.81 (s, 2H); ¹⁹F-NMR (376 MHz, CDCl₃) δ −152.01-−152.17 (m, 2F), −157.15 (t, J=21.6 Hz, 1F), −161.89-−162.14 (m, 2F); HRMS-ESI (m/z) calculated for C₁₆H₇F₅NO₄ [M+H]: 372.0290; found: 372.0280.

### Pentafluorophenyl 1-ethyl-7-methyl-4-oxo-1,4-dihydro-1,8-naphthyridine-3-carboxylate (28)

This compound was synthesized according to General Procedure A starting from 1-ethyl-7-methyl-4-oxo-1,4-dihydro-1,8-naphthyridine-3-carboxylic acid and pentafluorophenol. The preparative TLC was run with ethyl acetate/DCM 1:4. 245 mg (75%) of the product were obtained. ¹H-NMR (400 MHz, CDCl₃): δ 8.79 (s, 1H), 8.68 (d, J=8.1 Hz, 1H), 7.31 (d, J=8.1 Hz, 1H), 4.55 (q, J=7.2 Hz, 2H), 2.70 (s, 3H), 1.55 (t, J=7.2 Hz, 3H); ¹⁹F-NMR (376 MHz, CDCl₃) δ −152.27-−152.46 (m, 2F), −158.73 (t, J=21.5 Hz, 1F), −162.91-−163.10 (m, 2F); HRMS-ESI (m/z) calculated for C₁₈H₁₂F₅N₂O₃[M+H]: 399.0763; found: 399.0764.

### 2,4-Dinitrophenyl 3-(1,3-diphenyl-1H-pyrazol-4-yl)propanoate (29)

This compound was synthesized according to General Procedure A starting from 3-(1,3-diphenyl-1H-pyrazol-4-yl)propanoic acid and 2,4-dinitrophenol. The preparative TLC was run with ethyl acetate/n-hexane 2:3. A second preparative TLC was run with DCM/ethyl acetate 5:1. 142 mg (38%) of the product were obtained. ¹H-NMR (400 MHz, CDCl₃): δ 8.95 (d, J=2.7 Hz, 1H), 8.48 (dd, J=8.9, 2.7 Hz, 1H), 7.90 (s, 1H), 7.79-7.72 (m, 4H), 7.51-7.43 (m, 4H), 7.42-7.35 (m, 2H), 7.31-7.26 (m, 1H), 3.20 (t, J=7.4 Hz, 2H), 3.01 (t, J=7.4 Hz, 2H); ¹³C-NMR (100 MHz, CDCl₃): δ 169.73, 151.47, 148.50, 145.16, 141.69, 140.01, 133.45, 129.53, 129.16, 128.81, 128.16, 127.92, 126.75, 126.68, 126.43, 121.80, 118.88, 118.79, 34.64, 19.63; HRMS-ESI (m/z) calculated for C₂₄H₁₉N₄O₆ [M+H]: 459.1299; found: 459.1299.

### 2,4-Dinitrophenyl 2,2-diphenylacetate (30)

This compound was synthesized according to General Procedure B starting from 2,2-diphenylacetyl chloride and 2,4-dinitrophenol. The preparative TLC was run with n-hexane/DCM 2:3. The product was further purified by column chromatography using n-hexane/DCM 3:2. 114 mg (37%) of the product were obtained. ¹H-NMR (400 MHz, CDCl₃): δ 8.95 (d, J=2.7 Hz, 1H), 8.48 (dd, J=8.9, 2.7 Hz, 1H), 7.43-7.31 (m, 11H), 5.40 (s, 1H); HRMS-ESI (m/z) calculated for C₂₀H₁₄N₂NaO₆[M+Na]: 401.0744; found: 401.0746.

### 2,4-Dinitrophenyl 3,5-bis(trifluoromethyl)benzoate (31)

This compound was synthesized according to General Procedure B starting from 3,5-bis(trifluoromethyl)benzoyl chloride and 2,4-dinitrophenol. The preparative TLC was run with n-hexane/DCM 2:3. The product was further purified by column chromatography using n-hexane/DCM 3:2. 114 mg (33%) of the product were obtained. ¹H-NMR (400 MHz, CDCl₃): δ 9.09 (d, J=2.6 Hz, 1H), 8.68-8.60 (m, 3H), 8.22 (s, 1H), 7.67 (d, J=8.9 Hz, 1H); ¹⁹F-NMR (376 MHz, CDCl₃) δ −63.28 (s, 6F). ¹³C-NMR (100 MHz, CDCl₃): δ 161.40, 148.20, 145.83, 141.58, 133.11 (q, J=33.9 Hz), 130.81, 129.90, 129.61, 128.26, 126.79, 122.73 (q, J=273.9 Hz), 122.29; HRMS (m/z) calculated for C₁₅H₆F₆N₂NaO₆ [M+Na]: 447.0022; found: 447.0029.

### 2,4-Dinitrophenyl 2-(1-methyl-1H-indol-3-yl)acetate (32)

This compound was synthesized according to General Procedure A starting from 2-(1-methyl-1H-indol-3-yl)acetic acid and 2,4-dinitrophenol. The preparative TLC was run with DCM/n-hexane 2:1. 234 mg (54%) of the product were obtained. ¹H-NMR (400 MHz, CDCl₃): δ 8.94 (d, J=2.7 Hz, 1H), 8.45 (dd, J=8.9, 2.7 Hz, 1H), 7.65 (d, J=7.9 Hz, 1H), 7.40 (d, J=8.9 Hz, 1H), 7.34 (d, J=8.2 Hz, 1H), 7.27 (t, J=7.2 Hz, 1H), 7.17 (t, J=7.4 Hz, 2H), 4.15 (s, 2H), 3.80 (s, 3H); ¹³C-NMR (100 MHz, CDCl₃): δ 168.90, 148.96, 145.10, 141.75, 137.07, 129.04, 128.44, 127.59, 126.79, 122.21, 121.78, 119.71, 118.76, 109.65, 104.68, 32.95, 31.07; HRMS-ESI (m/z) calculated for C₁₇H₁₄N₃O₆ [M+H]: 356.0877; found: 356.0878.

### 2,4-Dinitrophenyl 3-(3,4,5-trimethoxyphenyl)propanoate (33)

This compound was synthesized according to General Procedure A starting from 3-(3,4,5-trimethoxyphenyl)propanoic acid and 2,4-dinitrophenol. The preparative TLC was run with ethyl acetate/n-hexane 2:3. 143 mg (43%) of the product were obtained. ¹H-NMR (400 MHz, CDCl₃): δ 8.97 (d, J=2.1 Hz, 1H), 8.52 (dd, J=9.0, 2.1 Hz, 1H), 7.40 (d, J=9.0 Hz, 1H), 6.47 (s, 2H), 3.87 (s, 6H), 3.84 (s, 3H), 3.08-2.98 (m, 4H); ¹³C-NMR (100 MHz, CDCl₃): δ 169.74, 153.49, 148.62, 145.22, 141.78, 136.86, 135.28, 129.19, 126.71, 121.85, 105.41, 60.99, 56.26, 35.48, 30.80; HRMS-ESI (m/z) calculated for C₁₈H₁₉N₂O₉ [M+H]: 407.1085; found: 407.1087.

### 1-Benzyl 4-(2,4-dinitrophenyl) piperidine-1,4-dicarboxylate (34)

This compound was synthesized according to General Procedure A starting from 1-((benzyloxy)carbonyl)piperidine-4-carboxylic acid and 2,4-dinitrophenol. The preparative TLC was run with ethyl acetates/DCM 1:9. 215 mg (61%) of the product were obtained. ¹H-NMR (400 MHz, CDCl₃): δ 8.97 (d, J=2.6 Hz, 1H), 8.52 (dd, J=8.9, 2.7 Hz, 1H), 7.46 (d, J=8.9 Hz, 1H), 7.39-7.29 (m, 5H), 5.15 (s, 2H), 4.21 (s, 2H), 3.02 (t, J=12.6 Hz, 2H), 2.87 (tt, J=11.0, 3.9 Hz, 1H), 2.17-2.05 (m, 2H), 1.92-1.77 (m, 2H); HRMS-ESI (m/z) calculated for C₂₀H₂₀N₃O₈[M+H]: 430.1245; found: 430.1243.

### 2,4-Dinitrophenyl quinoline-2-carboxylate (35)

This compound was synthesized according to General Procedure A starting from quinoline-2-carboxylic acid and 2,4-dinitrophenol. The preparative TLC was run with DCM. 25 mg (9%) of the product were obtained. ¹H-NMR (400 MHz, CDCl₃): δ 9.08 (d, J=2.6 Hz, 1H), 8.62 (dd, J=9.0, 2.7 Hz, 1H), 8.43 (d, J=8.5 Hz, 1H), 8.36 (d, J=8.6 Hz, 1H), 8.32 (d, J=8.5 Hz, 1H), 7.97 (d, J=8.2 Hz, 1H), 7.87 (t, J=7.7 Hz, 1H), 7.79-7.70 (m, 2H); HRMS-ESI (m/z) calculated for C₁₆H₁₀N₃O₆ [M+H]: 340.0564; found: 340.0565.

### 2,4-Dinitrophenyl 3-(7-fluoro-4-oxo-4H-chromen-3-yl)propanoate (36)

This compound was synthesized according to General Procedure A starting from 3-(7-fluoro-4-oxo-4H-chromen-3-yl)propanoic acid and 2,4-dinitrophenol. The preparative TLC was run with CHCl₃/acetone 95:5. 62 mg (19%) of the product were obtained. ¹H-NMR (400 MHz, CDCl₃): δ 8.97 (d, J=2.7 Hz, 1H), 8.54 (dd, J=8.9, 2.7 Hz, 1H), 7.97 (s, 1H), 7.89 (dd, J=8.2, 3.1 Hz, 1H), 7.54-7.47 (m, 2H), 7.46-7.40 (m, 1H), 3.12 (t, J=6.9 Hz, 2H), 2.93 (t, J=6.9 Hz, 2H); ¹⁹F-NMR (376 MHz, CDCl₃) δ −115.29 (s, 1F); HRMS-ESI (m/z) calculated for C₁₈H₁₂FN₂O₈ [M+H]: 403.0572; found: 403.0575.

### 2,4-Dinitrophenyl [1,1′-biphenyl]-4-carboxylate (37)

This compound was synthesized according to General Procedure A starting from 1,1′-biphenyl-4-carboxylic acid and 2,4-dinitrophenol. The preparative TLC was run with n-hexane/DCM 2:3. The product was further purified by column chromatography using n-hexane/DCM 3:2. 57 mg (19%) of the product were obtained. ¹H-NMR (400 MHz, CDCl₃): δ 9.02 (d, J=2.7 Hz, 1H), 8.59 (dd, J=8.9, 2.7 Hz, 1H), 8.26 (d, J=8.3 Hz, 2H), 7.78 (d, J=8.3 Hz, 2H), 7.70-7.64 (m, 3H), 7.51 (t, J=7.5 Hz, 2H), 7.45 (t, J=7.3 Hz, 1H); HRMS-ESI (m/z) calculated for C₁₉H₁₂N₂NaO₆ [M+Na]: 387.0588; found: 387.0588.

### 2,4-Dinitrophenyl 2-(adamantan-1-yl)acetate (38)

This compound was synthesized according to General Procedure A starting from 2-(adamantan-1-yl)acetic acid and 2,4-dinitrophenol. The preparative TLC was run with n-hexane/DCM 2:3. The product was further purified by column chromatography using n-hexane/DCM 3:2. 143 mg (48%) of the product were obtained. ¹H-NMR (400 MHz, CDCl₃): δ 8.93 (d, J=2.6 Hz, 1H), 8.50 (dd, J=9.0, 2.6 Hz, 1H), 7.47 (d, J=8.9 Hz, 1H), 2.45 (s, 2H), 2.03 (s, 3H), 1.81-1.63 (m, 12H); HRMS (m/z) calculated for C₁₈H₂₀N₂NaO₆ [M+Na]: 383.1213; found: 383.1204.

### 2,4-Dinitrophenyl 4-phenoxybenzoate (39)

This compound was synthesized according to General Procedure A starting from 4-phenoxybenzoic acid and 2,4-dinitrophenol. The preparative TLC was run with n-hexane/DCM 2:3. A second preparative TLC was run with n-hexane/ethyl acetate 6:1. 70 mg (22%) of the product were obtained. ¹H-NMR (400 MHz, CDCl₃): δ 9.00 (d, J=2.7 Hz, 1H), 8.56 (dd, J=9.0, 2.8 Hz, 1H), 8.18-8.12 (m, 2H), 7.65 (d, J=8.9 Hz, 1H), 7.44 (t, J=7.7 Hz, 2H), 7.28-7.22 (m, 1H), 7.12 (d, J=8.4 Hz, 2H), 7.07 (d, J=9.0 Hz, 2H); HRMS-ESI (m/z) calculated for C₁₉H₁₂N₂NaO₇ [M+Na]: 403.0537; found: 403.0537.

### 2,4-Dinitrophenyl 2-((3-(trifluoromethyl)phenyl)amino)benzoate (40)

This compound was synthesized according to General Procedure A starting from 2-((3-(trifluoromethyl)phenyl)amino)benzoic acid and 2,4-dinitrophenol. The preparative TLC was run with DCM/n-hexane 3:2. 254 mg (69%) of the product were obtained. ¹H-NMR (400 MHz, CDCl₃): δ 9.11 (s, 1H), 9.01 (d, J=2.7 Hz, 1H), 8.57 (dd, J=8.9, 2.7 Hz, 1H), 8.20 (dd, J=8.1, 1.7 Hz, 1H), 7.64 (d, J=8.9 Hz, 1H), 7.53-7.45 (m, 3H), 7.44-7.36 (m, 2H), 7.28 (d, J=8.6 Hz, 1H), 6.91 (t, J=7.4 Hz, 1H); ¹⁹F-NMR (376 MHz, CDCl₃) δ −63.09 (s, 3F); ¹³C-NMR (100 MHz, CDCl₃): δ 165.12, 148.80, 148.68, 145.19, 142.10, 140.65, 136.53, 132.68, 132.15 (q, J=32.8 Hz), 130.25, 129.08, 127.01, 125.91, 123.93 (q, 272.9 Hz), 121.86, 120.94 (q, J=3.9 Hz), 119.40 (q, J=3.8 Hz), 118.72, 114.35, 109.65; HRMS-ESI (m/z) calculated for C₂₀H₁₃F₃N₃O₆ [M+H]: 448.0751; found: 448.0753.

### 2,4-Dinitrophenyl 4-((tert-butoxycarbonyl)amino)butanoate (41)

This compound was synthesized according to General Procedure A starting from 4-((tert-butoxycarbonyl)amino)butanoic acid and 2,4-dinitrophenol. The preparative TLC was run with ethyl acetate/DCM 1:9. 126 mg (42%) of the product were obtained. ¹H-NMR (400 MHz, CDCl₃): δ 8.96 (d, J=2.6 Hz, 1H), 8.52 (dd, J=8.9, 2.7 Hz, 1H), 7.54 (d, J=8.9 Hz, 1H), 4.68 (s, 1H), 3.27 (q, J=6.6 Hz, 2H), 2.75 (t, J=7.2 Hz, 2H), 1.96 (p, J=7.0 Hz, 2H), 1.45 (s, 9H); HRMS-ESI (m/z) calculated for C₁₅H₂₀N₃O₈ [M+H]: 370.1245; found: 370.1244.

### 2,4-Dinitrophenyl 2,2,2-triphenylacetate (42)

This compound was synthesized according to General Procedure A starting from 2,2,2-triphenylacetic acid and 2,4-dinitrophenol. The preparative TLC was run with CHCl₃/acetone 95:5. A second preparative TLC was run with the same solvent mixture. 116 mg (31%) of the product were obtained. ¹H-NMR (400 MHz, CDCl₃): δ 8.89 (d, J=2.7 Hz, 1H), 8.40 (dd, J=9.0, 2.7 Hz, 1H), 7.42-7.29 (m, 15H), 7.02 (d, J=9.0 Hz, 1H); HRMS-ESI (m/z) calculated for C₂₆H₁₈N₂NaO₆ [M+Na]: 477.1057; found: 477.1060.

### 2,4-Dinitrophenyl acetate (43)

This compound was synthesized according to General Procedure B starting from acetyl chloride and 2,4-dinitrophenol. The preparative TLC was run with DCM/n-hexane 2:1. 57 mg (31%) of the product were obtained. ¹H-NMR (400 MHz, CDCl₃): δ 8.97 (d, J=2.7 Hz, 1H), 8.52 (dd, J=8.9, 2.7 Hz, 1H), 7.48 (d, J=8.9 Hz, 1H), 2.43 (s, 3H); HRMS (m/z) calculated for C₈H₆N₂NaO₆ [M+Na]: 249.0118; found: 249.0116.

### 2,4-Dinitrophenyl 4-cyanobenzoate (44)

This compound was synthesized according to General Procedure B starting from 4-cyanobenzoyl chloride and 2,4-dinitrophenol. Instead of a preparative TLC, the reaction was purified using column chromatography with DCM/n-hexane 4:1. 104 mg (41%) of the product were obtained. ¹H-NMR (400 MHz, CDCl₃): δ 9.05 (d, J=2.8 Hz, 1H), 8.61 (dd, J=8.9, 2.7 Hz, 1H), 8.31 (d, J=8.3 Hz, 2H), 7.87 (d, J=8.3 Hz, 2H), 7.67 (d, J=8.9 Hz, 1H); HRMS (m/z) calculated for C₁₄H₈N₃O₆[M+H]: 314.0408; found: 314.0406.

### 2,4-Dinitrophenyl 3-(benzo[d][1,3]dioxol-5-yl)propanoate (45)

This compound was synthesized according to General Procedure A starting from 3-(benzo[d][1,3]dioxol-5-yl)propanoic acid and 2,4-dinitrophenol. The preparative TLC was run with ethyl acetate/n-hexane 2:3. A second preparative TLC was run with DCM/ethyl acetate 5:1. 108 mg (37%) of the product were obtained. ¹H-NMR (400 MHz, CDCl₃): δ 8.96 (d, J=2.7 Hz, 1H), 8.50 (dd, J=8.9, 2.7 Hz, 1H), 7.40 (d, J=8.9 Hz, 1H), 6.80-6.68 (m, 3H), 5.95 (s, 2H), 3.06-2.94 (m, 4H); HRMS-ESI (m/z) calculated for C₁₆H₁₂N₂NaO₈ [M+Na]: 383.0486; found: 383.0488.

### 3,5-Bis(trifluoromethyl)benzoic acid NHS ester (46)

This compound was synthesized according to General Procedure B starting from 3,5-bis(trifluoromethyl)benzoyl chloride and N-hydroxysuccinimide. The preparative TLC was run with DCM. 169 mg (58%) of the product were obtained. ¹H-NMR (400 MHz, CDCl₃): δ 8.58 (s, 2H), 8.19 (s, 1H), 2.95 (s, 4H); ¹⁹F-NMR (376 MHz, CDCl₃) δ −63.38 (s, 6F); HRMS-ESI (m/z) calculated for C₁₃H₈F₆NO₄ [M+H]: 356.0352; found: 356.0352.

### 2,3,5,6-Tetrafluoro-4-(trifluoromethyl)phenyl 3,5-bis(trifluoromethyl)benzoate (47)

This compound was synthesized according to General Procedure B starting from 3,5-bis(trifluoromethyl)benzoyl chloride and 2,3,5,6-tetrafluoro-4-(trifluoromethyl)phenol. The preparative TLC was run with n-hexane/DCM 2:1. 283 mg (73%) of the product were obtained. ¹H-NMR (400 MHz, CDCl₃): δ 8.65 (s, 2H), 8.23 (s, 1H); ¹⁹F-NMR (376 MHz, CDCl₃) δ −56.38 (t, J=22.0 Hz, 3F), −63.36 (s, 6F), −139.52-−139.92 (m, 2F), −149.93-−150.20 (m, 2F); ¹³C-NMR (100 MHz, CDCl₃): δ 159.83, 144.89 (d, J=265.2 Hz), 141.33 (d, J=249.4 Hz), 133.28 (q, J=34.9 Hz), 132.07, 130.94, 129.06, 128.48, 122.71 (q, J=271.9 Hz), 120.77 (q, J=276.2 Hz), 108.64. HRMS could not be obtained.

### 2,3,5,6-Tetrafluorophenyl 3,5-bis(trifluoromethyl)benzoate (48)

This compound was synthesized according to General Procedure B starting from 3,5-bis(trifluoromethyl)benzoyl chloride and 2,3,5,6-tetrafluorophenol. The preparative TLC was run with n-hexane/DCM 2:1. 285 mg (86%) of the product were obtained. ¹H-NMR (400 MHz, CDCl₃): δ 8.66 (s, 2H), 8.21 (s, 1H), 7.11 (tt, J=9.8, 6.9 Hz, 1H); ¹⁹F-NMR (376 MHz, CDCl₃) δ −63.31 (s, 6F), −138.31-−138.44 (m, 2F), −152.69-−152.82 (m, 2F); HRMS (m/z) calculated for C₁₅H₅F₁₀O₂[M+H]: 407.0124; found: 407.0125.

### N-Methoxycarbonyl-pyrazole-1-carboxamidine (49a)

2.94 g (20.1 mmol, 1 eq.) pyrazole-1-carboxamidine hydrochloride were dissolved in 20 ml DCM and 10.2 ml (7.55 g, 58 mmol, 2.9 eq.) DIPEA. 1.55 ml (1.9 g, 20.1 mmol, 1 eq.) methyl chloroformate were added and the solution was stirred at room temperature for 12 h. The product was purified by column chromatography using DCM as the eluent to give 2.47 g (73%) of the product. ¹H-NMR (400 MHz, CDCl₃): δ 9.04 (s, 1H), 8.44 (d, J=2.8 Hz, 1H), 7.70 (d, J=1.0 Hz, 1H), 7.65 (s, 1H), 6.43 (dd, J=2.8, 1.0 Hz, 1H), 3.81 (s, 3H). ¹³C-NMR (100 MHz, CDCl₃): δ 164.61, 155.45, 143.82, 128.88, 109.48, 53.02; HRMS (m/z) calculated for C₆H₉N₄O₂[M+H]: 169.0720; found: 169.0723.

### N-Methoxycarbonyl-N′-9-fluorenylmethoxycarbonyl-pyrazole-1-carboxamidine (49)

100 mg (0.6 mmol, 1 eq.) 49a were dissolved in 4 ml anhydrous THF and cooled to 0° C. To this, 35 mg sodium hydride (60% in mineral oil, 0.88 mmol, 1.5 eq.) were added and the mixture was stirred at 0° C. for 1 h. 171 mg Fmoc-C1 (0.66 mmol, 1.1 eq.) were added and the reaction was warmed to room temperature over night and directly loaded onto a preparative TLC. The TLC was run with Et₂O/hexanes 2:1. A second preparative TLC was run with ethyl acetate/n-hexane 1:1. 56 mg (24%) of the product were obtained as a mixture of two tautomers (ratio of about 1.1:0.9). ¹H-NMR (400 MHz, CDCl₃): δ 9.47-9.27 (m, 1H), 8.38 (s, 0.55H), 8.32 (s, 0.45H), 7.78 (d, J=7.6 Hz, 2H), 7.73-7.67 (m, 2H), 7.65-7.56 (m, 1H), 7.48-7.37 (m, 2H), 7.37-7.28 (m, 2H), 6.51 (s, 1H), 4.56-4.46 (m, 2H), 4.45-4.36 (m, 0.55H), 4.34-4.25 (m, 0.45H), 3.84 (s, 1.35H), 3.74 (s, 1.65H); ¹³C-NMR (100 MHz, CDCl₃): δ 159.07, 158.54, 151.32, 150.88, 144.22, 143.21, 141.42, 138.53, 138.40, 129.10, 128.16, 127.78, 127.40, 127.19, 125.56, 125.15, 120.29, 120.04, 110.55, 69.01, 68.75, 53.86, 46.94, 46.71; HRMS (m/z) calculated for C₂₁H₁₉N₄O₄ [M+H]: 391.1401; found: 391.1409.

A Chemical Proteomic Method for Assessing Lysine Reactivity

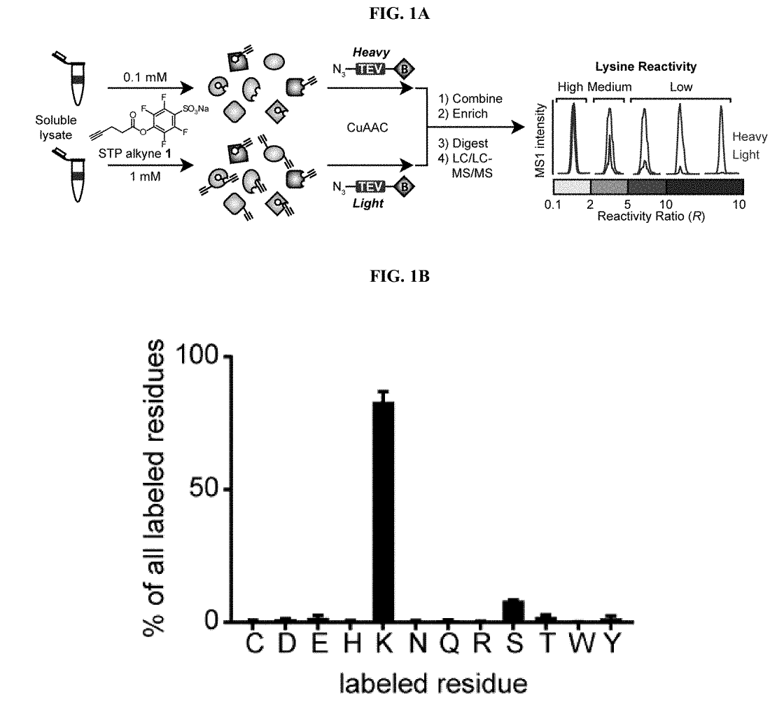

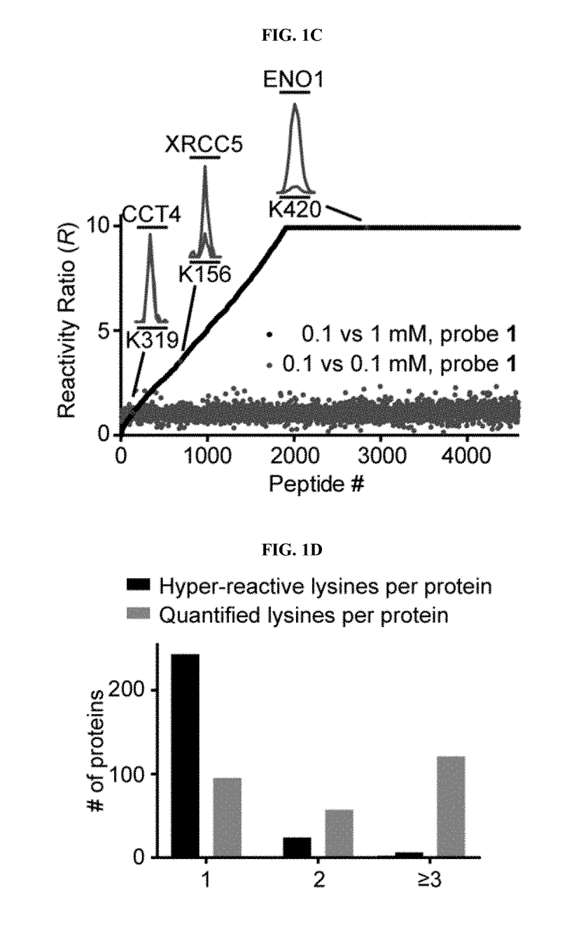

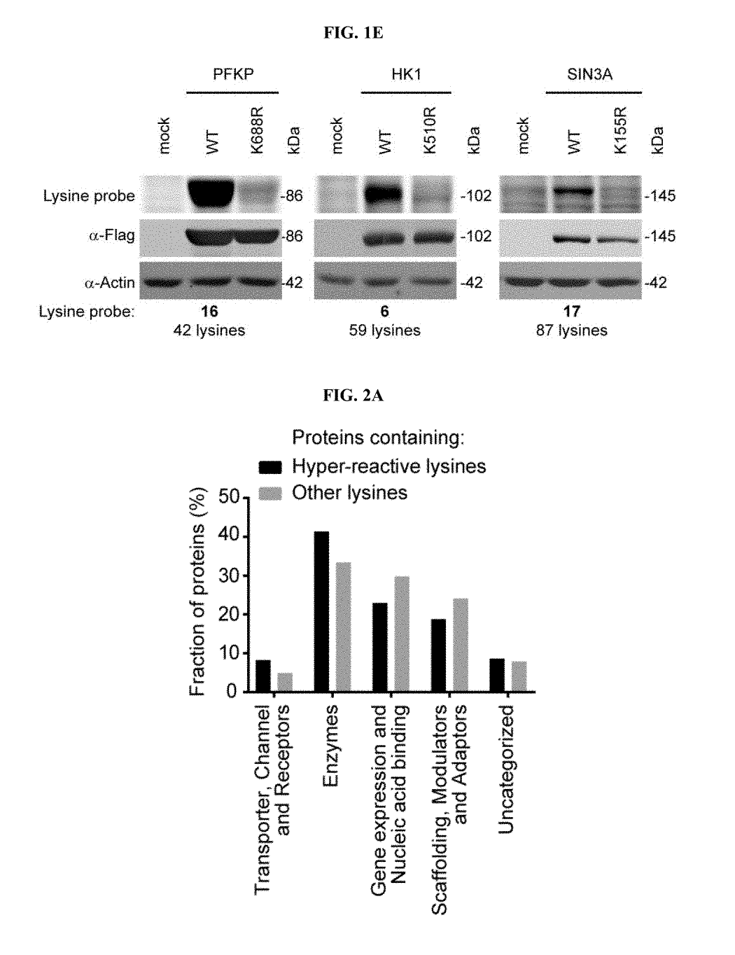

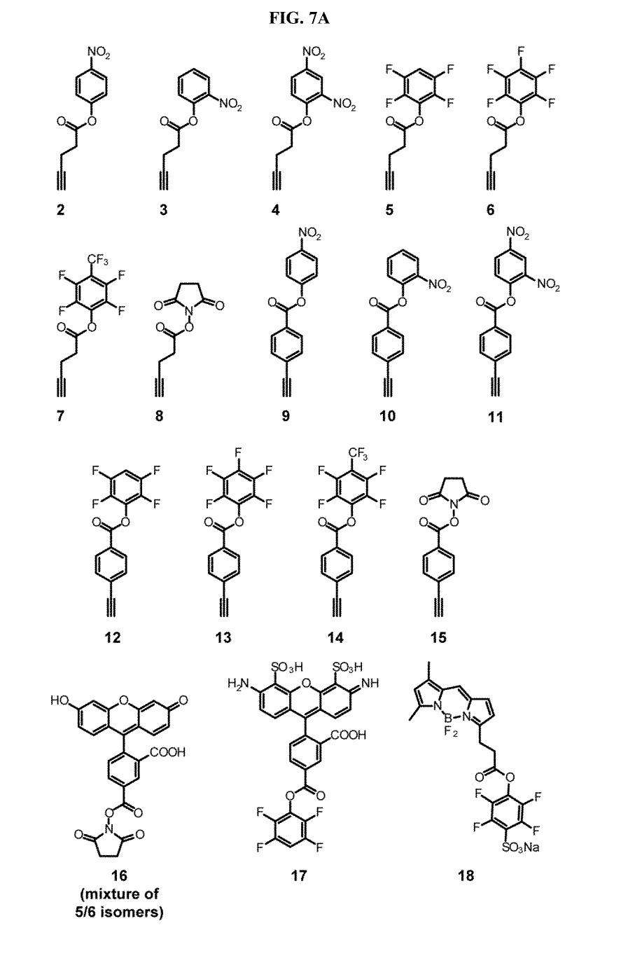

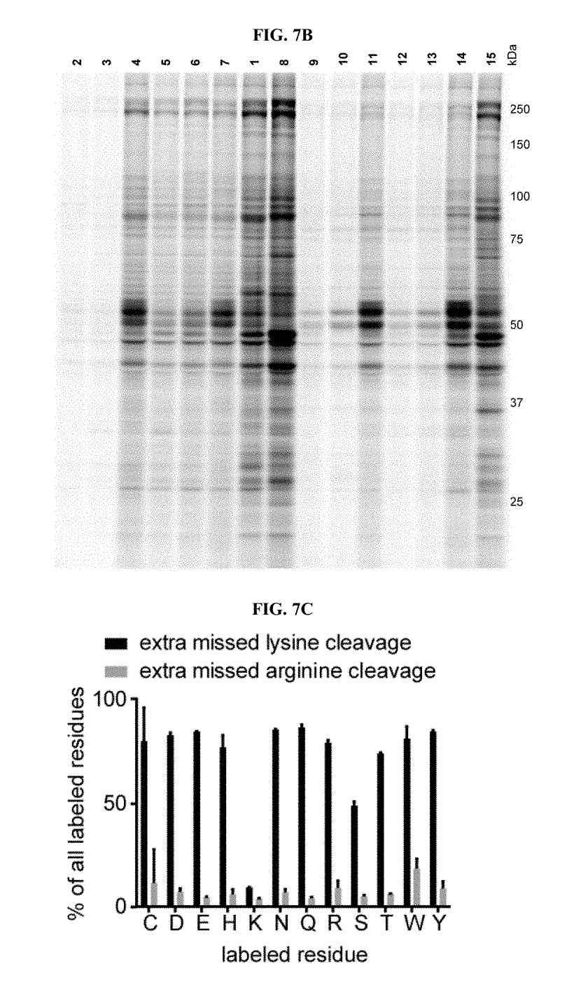

In some instances, described herein is an illustrative example on global profiling of lysine reactivity (FIG. 1A). In some instances, activated esters show preferred reactivity with amines relative to other reactive compound classes, display good solubility, and form stable, structurally simple adducts with proteinaceous lysines for characterization by MS methods. In an initial screen of alkyne-modified ester probes (1-15, FIG. 7A), it was found that sulfotetrafluorophenyl (STP) and N-hydroxysuccinimide esters showed proteomic reactivity as evaluated by copper-catalyzed azide-alkyne cycloaddition (CuAAC, or click chemistry) to a rhodamine-azide tag, SDS-PAGE, and in-gel fluorescence scanning (FIG. 7B). Considering that tetrafluorophenyl esters are more stable in aqueous solution compared to NHS esters, STP-alkyne 1 was selected as a probe for proteomic profiling of lysine reactivity.

To assess the scope and selectivity with which 1 reacted with lysine residues in human cell proteomes, initial isoTOP-ABPP experiments were performed as follows. Two equal amounts of the soluble proteome of the human breast cancer cell line MDA-MB-231 (0.75 mg of protein per sample) were treated with 1 (100 μM, 1 h), and then conjugated by copper-catalyzed azide-alkyne cycloaddition (CuAAC) to isotopically differentiated TEV-cleavable, azide-biotin tags (heavy and light, respectively). The heavy and light-tagged samples were then combined, and 1-labeled proteins enriched by streptavidin and proteolytically digested sequentially with trypsin and TEV protease (to release 1-labeled tryptic peptides from the streptavidin support), furnishing isotopic (heavy/light) peptide pairs that were analyzed by multidimensional liquid chromatography-MS(LC/LC-MS/MS). Measurement of the MS1 chromatographic peak ratios for light/heavy peptide pairs provided an isoTOP-ABPP ratio or R value, which centered on about 1.0 for the more than 5000 probe 1-labeled peptides quantified in this initial study. Tandem MS and differential modification analysis were then used to assign the amino acid residue labeled by 1 within each tryptic peptide. In this pilot experiment, >52% of 1-labeled peptides were assigned as being uniquely modified on lysine residues, with 54% of the remaining 1-labeled peptides being assigned with lysine modifications as well as alternative residue modifications. Because lysine modification creates a missed trypsin cleavage site, the fractions of alternative amino-acid modification assignments were further assessed for their occurrence on peptides harboring a missed lysine cleavage site. It was found that most of the predicted non-lysine modifications for 1 occurred on peptides with missed lysine cleavage sites FIG. 7C), indicating that they likely represent mis-assignments of reactivity events that actually occurred on lysine. Once the isoTOP-ABPP data were filtered to remove peptide assignments with unmodified, missed lysine cleavage events, lysine accounted for the vast majority of all assignments for probe 1 modification (FIG. 1B). The remaining alternative probe 1 modifications were mostly assigned to serine (about 8% of the total 1-labeled peptides), and these occurred on fully digested tryptic peptides (FIG. 1B), likely designating them as authentic modifications. These results, taken together, indicate that 1 shows broad reactivity and good selectivity for lysine residues in the human proteome.

Quantitative Profiling of Lysine Reactivity in Human Cell Proteomes

Previous isoTOP-ABPP studies have shown that the human proteome possesses a specialized set of cysteine residues that show heightened reactivity with electrophilic small molecules and are enriched in functional residues (e.g., catalytic residues, redox-active residues) compared to bulk cysteine content. Here, the intrinsic reactivity of lysine residues was assessed in human cell proteomes. In brief, proteomes from three human cancer cell lines were treated (MDA-MB-231, Ramos, and Jurkat cells) with low vs high concentrations of probe 1 (0.1 vs 1 mM, n=4 per group) for 1 h and then analyzed the samples by isoTOP-ABPP, wherein high, medium, and low reactivity lysines were distinguished by their respective isotopic ratio values (R_{10:1}<2, 2<R_{10:1}<5, R_{10:1}>5, respectively). To minimize false quantification events, it was also required that lysines were detected in control (0.1 vs 0.1 mM) experiments with R_{1:1} values of about 1.0.

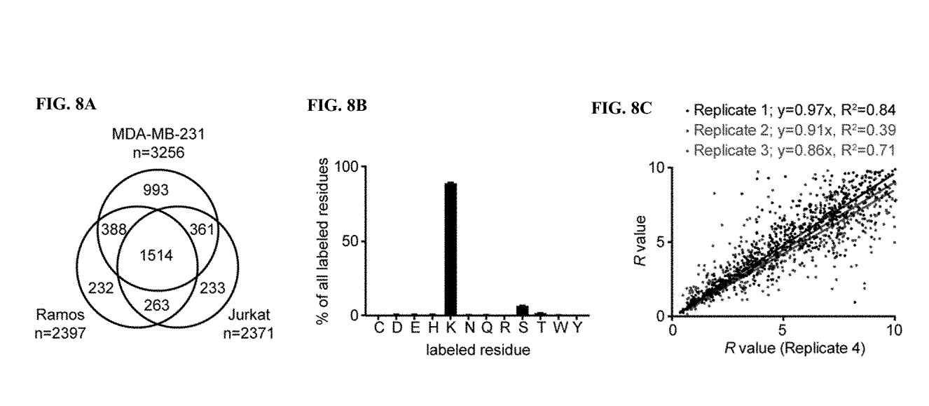

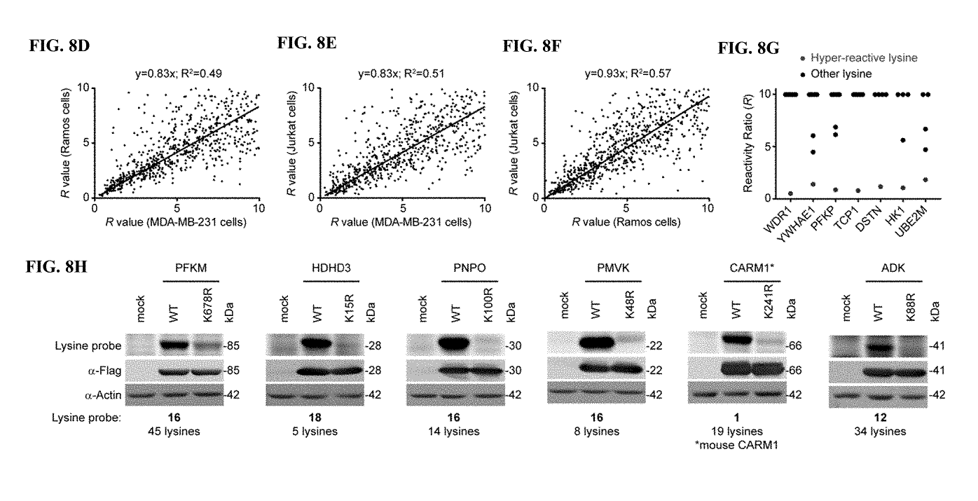

On average, the reactivity of about 1400 lysine residues was quantified per experiment, and, in total, about 4000 lysine residues were assessed for intrinsic reactivity across the three tested cell lines (FIG. 8A). Probe 1 also maintained excellent selectivity for lysine modification over other amino acids in these experiments using higher (1 mM) concentrations of the probe (FIG. 8B). The reactivity values for individual lysines were generally consistent for replicate experiments performed within the same cell line (FIG. 8C) and for experiments performed in different cell lines (FIGS. 8D-F), supporting the robustness of the isoTOP-ABPP method and suggesting that the reactivity of most lysine residues is an intrinsic feature that is preserved in different cell contexts.

The majority of quantified lysines showed strong, concentration-dependent increases in reactivity with probe 1, indicative of residues with low intrinsic reactivity (i.e., >50% of all quantified lysines showed R_{10:1} values=10) (FIG. 1C). In contrast, a rare subset of the quantified lysines (<10%, or 310 total residues) exhibited heightened (hyper-) reactivity with probe 1 (R_{10:1} values <2) (FIG. 1C). Most proteins contained only one hyper-reactive lysine among several quantified lysines (FIG. 1D). The atypical hyper-reactivity of these lysines was further supported by comparing their R_{10:1} values to those of other lysines quantified on the same protein (FIG. 8G). It was confirmed that the lysine hyper-reactivity determinations made by isoTOP-ABPP by recombinantly expressing wild type and lysine-to-arginine mutant proteins and comparing their reactivity by gel-based ABPP using fluorescent or alkyne-tagged activated ester probes (FIG. 8A). Each protein examined showed strong labeling with activated ester probes and the labeling of one or more of these probes was generally blocked, in many cases completely, by mutation of the hyper-reactive lysine to arginine (FIG. 1E, FIG. 8H, and Table 2). Considering that there were, on average, 30 lysine residues per examined protein, the blockade of activated ester probe reactivity by mutation of a single lysine in each protein underscores the unusual hyper-reactivity of these residues.

Features of Hyper-Reactive Lysines

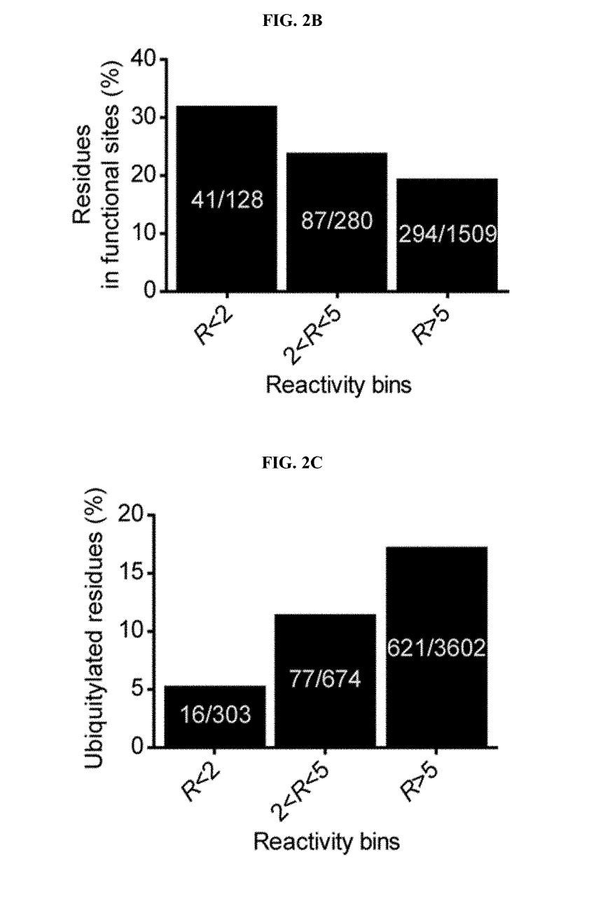

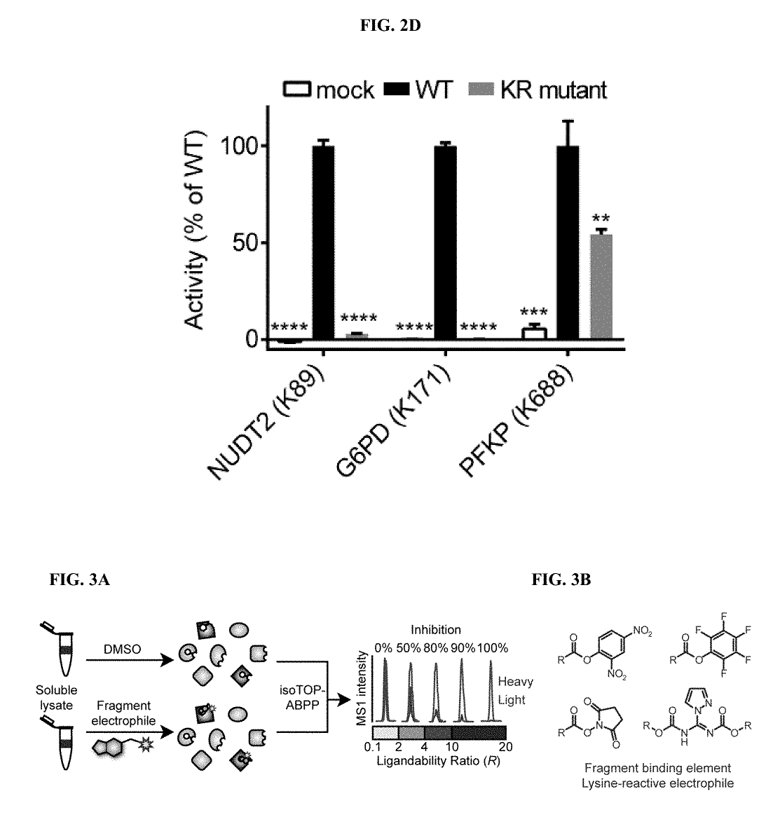

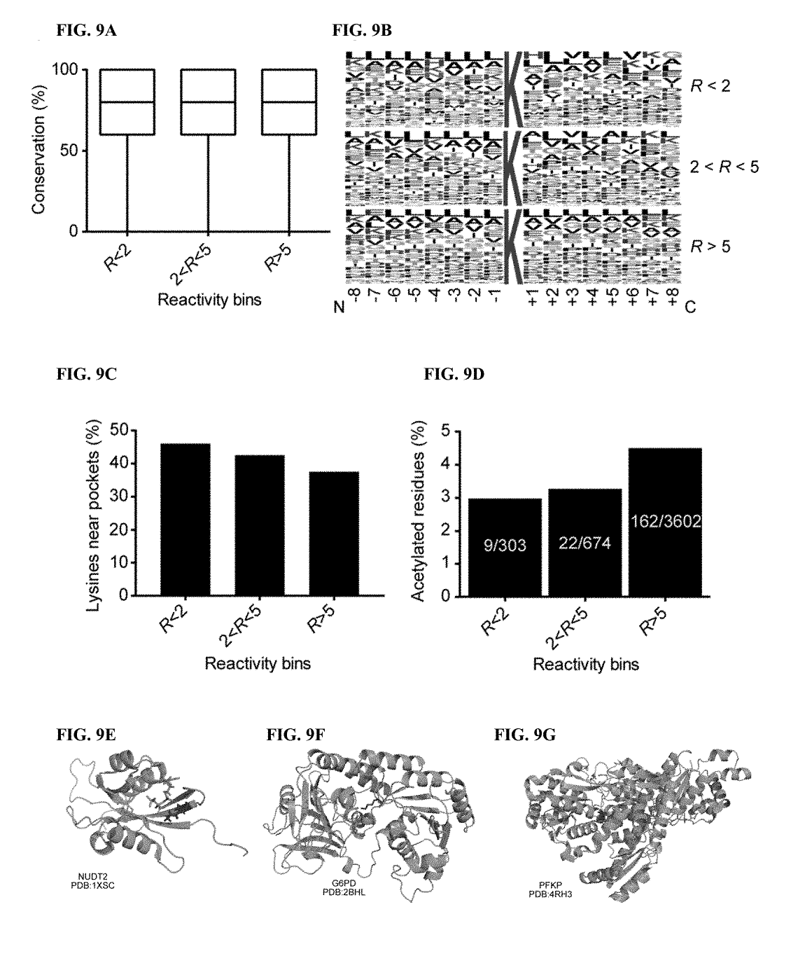

Hyper-reactive lysines were found on proteins from all major classes and showed a similar distribution to less reactive lysines (FIG. 2A). Hyper-reactive lysines were not, as a group, more conserved across organisms than lysines of lower reactivity, although this analysis proved complicated to interpret due to the high median conservation (about 80%) of all 1-labeled lysines across the species examined (H. sapiens, M. musculus, X. laevis, D. malanogaster, C. elegans and D. rerio) (FIG. 9A). The primary sequence surrounding hyper-reactive lysines also did not show evidence of any obvious conserved motifs (FIG. 9B), indicating that higher-order structural features in proteins are likely imparting enhanced reactivity on these lysines. Consistent with this hypothesis, the frequency of lysines found in functional sites on proteins (e.g., enzyme active sites, ligand-binding sites), as assessed by analysis of three-dimensional protein structures, was positively correlated with reactivity (FIG. 2B). Protein pockets of uncharacterized function (as defined by AutoSite analysis of protein structures) also contained a greater percentage of hyper-reactive lysines compared to less reactive lysines (FIG. 9C). Interestingly, it was observed a striking inverse correlation between lysine reactivity and evidence of ubiquitylation as reported in the PhosphoSitePlus® database, (FIG. 2C), and a similar, albeit more tempered trend was found for lysine acetylation (FIG. 9D). These data, taken together, indicate that the localization of lysines to pockets on proteins may represent a prevalent mechanism for conferring heightened reactivity, and such distributions may further hinder post-translational modification of the lysines possibly due to limited surface exposure.

It was examined whether some of the hyper-reactive lysines located in functional pockets contributed to protein activity. NUDT2, which is a diadenosine tetraphosphate hydrolase implicated in cancer and immune cell metabolism, possesses a hyper-reactive lysine (K89) that is highly conserved and predicted, based on an NMR structure of NUDT2, to coordinate alpha-phosphate substrate binding. It was found that mutation of K89 to arginine dramatically reduced the hydrolytic activity of NUDT2 (FIG. 2D). A similar disruption of catalysis was observed by mutation of the conserved, hyper-reactive lysine (K171) in the pentose phosphate pathway enzyme glucose 6-phosphate 1-dehydrogenase (G6PD) (FIG. 2D). Both K89 of NUDT2 and K171 of G6PD are active-site residues (FIG. 9E and FIG. 9F), and it was therefore wondered whether hyper-reactive lysines located in potential allosteric pockets might also affect enzyme function. As a case study, the hyper-reactive lysine (K688) in platelet-type phosphofructokinase (PFKP) was examined, which is located in an allosteric pocket >22 angstroms away from the active site (FIG. 9G). Mutation of K688 to arginine in PFKP produced a partial, but significant reduction in PFKP activity (FIG. 2D), pointing to a role for this lysine in allosteric regulation of PFKP function.

Quantitative Profiling of Lysine Ligandability in Human Cell Proteomes

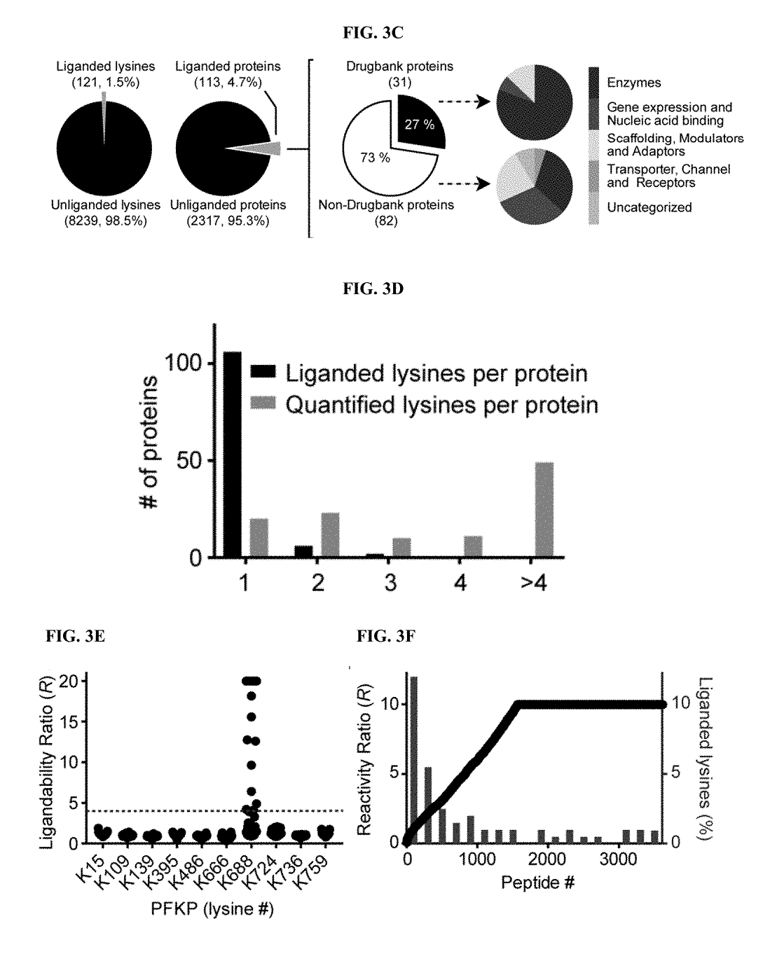

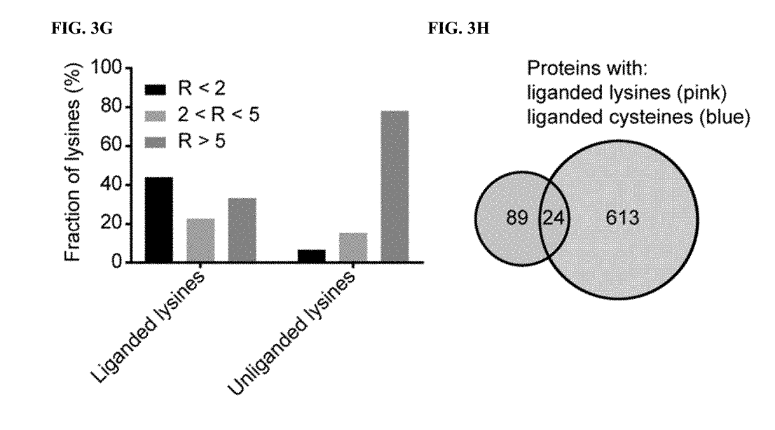

IsoTOP-ABPP methods have recently been used to assess the global reactivity of small-molecule electrophilic fragments with cysteines residues in human cell proteomes, leading to the discovery of hundreds of fragment-cysteine interactions. These “ligandable” cysteines were found in a diverse array of proteins, including those historically considered challenging to target with small molecules. Interested in more broadly assessing the ligandability potential of lysines in the human proteome, isoTOP-ABPP in a “competitive” format was applied (FIG. 3A), where human cell proteomes were pre-treated with a small library (about 30 member) of amine-reactive electrophilic fragments (activated esters, such as pentafluorophenyl-(19-28), dinitrophenyl-(29-45), and NHS esters (46), and N,N′-diacyl-pyrazolecarboxamidines (49,50), as well as one non-electrophilic control compound 51 (FIG. 3B, FIG. 10A, and FIG. 10B) or DMSO control, followed by exposure to probe 1. Fragment-sensitive lysines were identified as those showing substantial reductions (≥75%) in enrichment by 1 in the presence of one or more fragments compared to the DMSO control (R values ≥4 for DMSO/fragment).

Fragments were tested at 50-100 μM in duplicate for competitive blockade of reactivity of probe 1 (100 μM) with lysines in the human breast cancer cell MDA-MB-231 proteome. On average, >2700 lysines per dataset were quantified and, in aggregate, >8,000 lysines from 2,430 proteins across all datasets (FIG. 3C and Tables 1 and 3). Each lysine was quantified, on average, in 24 individual experiments (FIG. 10C and Tables 1 and 3), providing a good initial assessment of ligandability potential. An additional set of stringent data filtration criteria was implemented to limit false positive assignments of fragment-lysine interactions. In total, 121 liganded lysines in 113 proteins were identified (FIG. 3C). On average, about four lysines per protein that reacted with probe 1 were quantified (FIG. 3D), indicating that ligandability was a rare feature. A striking example is PFKP, where a single liganded lysine was identified—the aforementioned K688 that resides in an allosteric pocket—along with nine additional quantified lysines that were well-represented in the competitive isoTOP-ABPP experiments, but showed no evidence of ligandability (FIG. 3E). Likewise, hexokinase-1 (HK1) possessed a single liganded lysine K510 among six quantified lysines (FIG. 10D). The majority of proteins harboring liganded lysines were not found in DrugBank (73%; FIG. 3C), and these proteins showed much broader class distribution than the smaller fraction of DrugBank proteins containing liganded lysines (27%), which were mostly enzymes (FIG. 3C). Prominent sub-groups of non-Drugbank proteins with liganded lysines included transcription factors and scaffolding proteins (FIG. 3C), which are considered challenging to target with small molecules.

Hyper-reactive lysines showed greater ligandability compared to less reactive lysines, although many liganded lysines were also found in the latter group (R_{10:1}>2.0; FIG. 3F, FIG. 3G). Of note, only a small fraction (about 20%) of proteins with liganded lysines were found to contain liganded cysteines in a previous study (Backus, et al., “Proteome-wide covalent ligand discovery in native biological systems,” Nature 534, 570-574 (2016)) (FIG. 3H). These results, taken together, indicate that fragment electrophile interactions with lysines depend on both reactivity and recognition and canvas a distinct and complementary portion of the human proteome compared to covalent chemistries targeting other nucleophilic amino acids.

SAR Analysis of Lysine-Fragment Electrophile Interactions

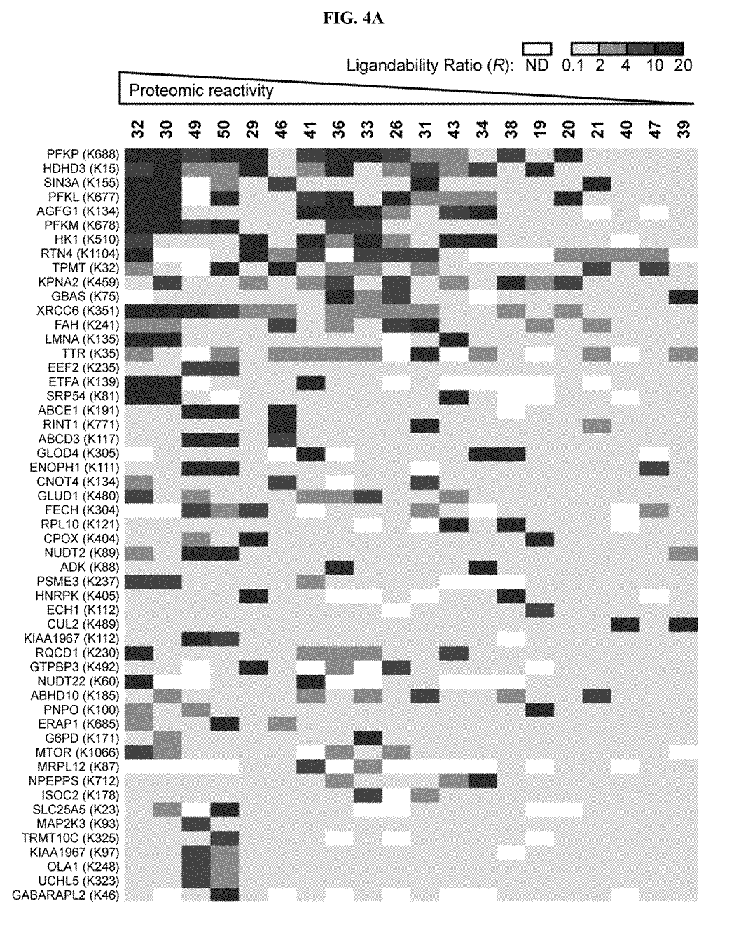

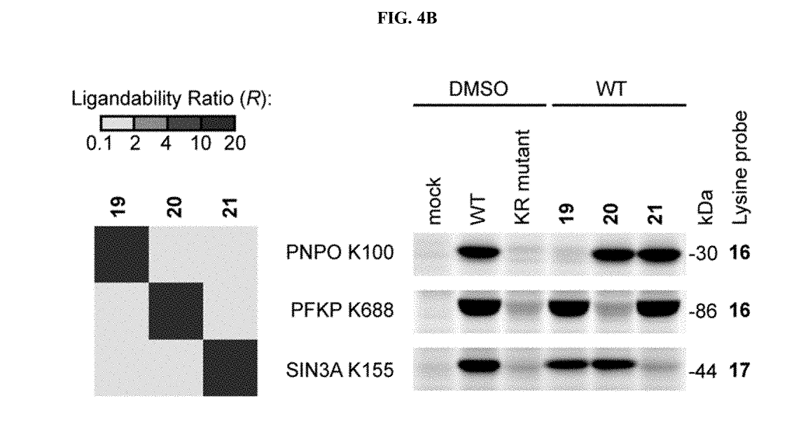

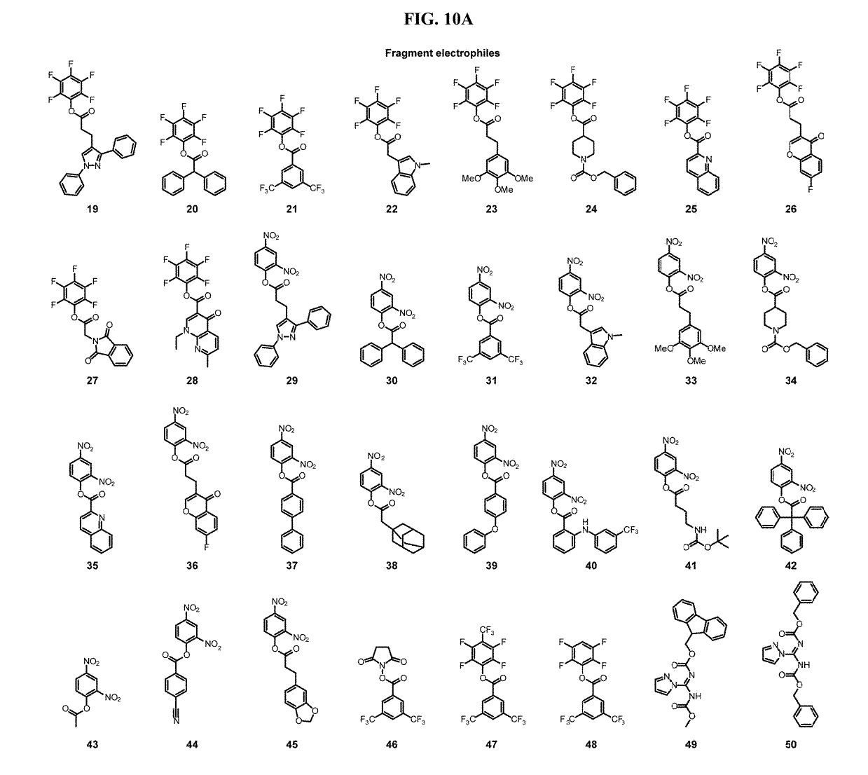

Most of the liganded lysines (69%) interacted with a limited fraction (<10%) of the tested fragment electrophiles, although a small subset of lysines (8%) was targeted by a substantial portion of the compounds (≥25%) (FIG. 11A). Conversely, the fragment electrophiles showed large differences in proteomic reactivity towards lysines (FIG. 11B), ranging from 1% to 35% of the liganded residues (FIG. 11C). No lysine reactivity was observed for the non-electrophilic control fragment 51 (FIGS. 10B and 11B,C). The dinitrophenyl esters showed somewhat greater overall reactivity compared to the corresponding pentafluorophenyl esters (FIG. 11B-D). Despite these general trends, individual lysines displayed markedly distinct structure-activity relationships (SARs) that, in some cases, directly opposed the overall reactivity profiles of the fragment electrophile library (FIG. 4A and Tables 1 and 3). The hyper-reactive lysine K35 in the hormone-binding protein transthyretin TTR, for instance, which has previously been shown to be modified selectively in human plasma by activated (thio)ester and sulfonyl fluoride ligands, was preferentially targeted by the dinitrophenyl ester fragment 31 over fragments that showed much greater proteome-wide reactivity (e.g., 29 and 30) (FIG. 10A and FIG. 11B, C). Further evidence that recognition events make substantive contributions to fragment-lysine interactions is reflected in the distinct lysine reactivity profiles displayed by fragment electrophiles bearing a common leaving group (FIG. 4B, left panel). These SAR assignments were confirmed by gel-based ABPP with recombinantly expressed proteins (FIG. 4B, right panels, and FIG. 11E). The identity of the leaving group of activated ester fragments also influenced reactivity, as reflected by a subset of lysines that were preferentially liganded by pentafluorophenyl or dinitrophenyl esters bearing the same recognition group (FIG. 11F). The most distinctive lysine reactivity profiles were observed for the N,N′-diacyl-pyrazolecarboxamidine fragments 49 and 50, which, despite sharing several targets with activated esters, also reacted with 15 lysines in human cell proteomes that showed negligible cross-reactivity with activated esters (see representative proteins at the bottom of FIG. 4A and Tables 1 and 3). The reactivity of one of these lysines (K89 of NUDT2) with N,N′-diacyl-pyrazolecarboxamidine fragments was confirmed by recombinant expression of the parent protein and competitive gel-based ABPP (FIG. 1G).

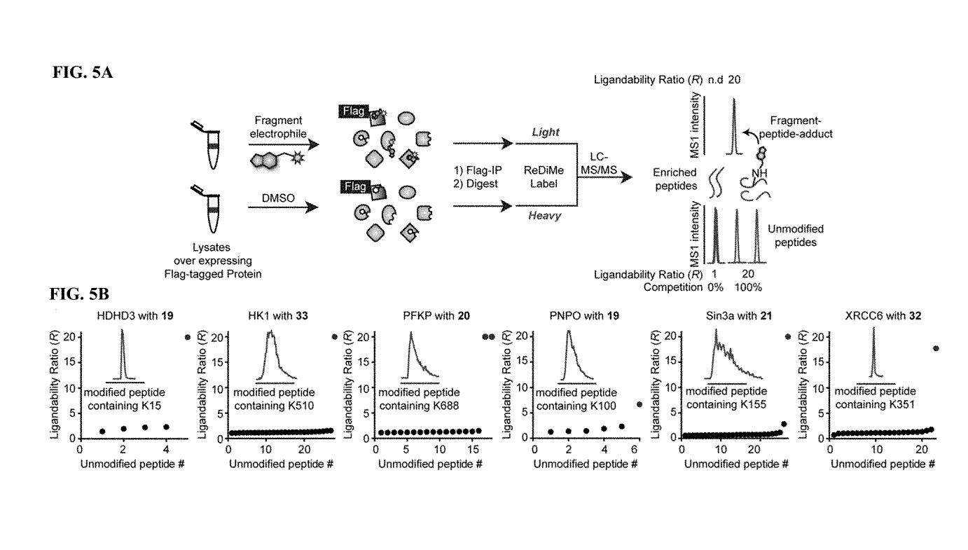

Because the isoTOP-ABPP platform indirectly reads out ligand interactions by competitive displacement of a broad, amino acid-reactive probe (e.g., probe 1 for lysines), it was sought to confirm these interactions by direct detection of fragment-lysine adducts. For this purpose, a quantitative, MS-based platform was developed that simultaneously measures both fragment electrophile modification of lysines in individual proteins and the fractional occupancy of these reactions (FIG. 5A). Proteins containing liganded lysines discovered by isoTOP-ABPP were produced with a Flag epitope tag in HEK 293T cells by transient transfection, and the transfected cell lysates were then treated with fragment electrophiles or DMSO and the proteins enriched by anti-Flag immunoprecipitation, proteolytically digested, isotopically labeled by reductive dimethylation (ReDiMe) with light or heavy formaldehyde (fragment- and DMSO-treated samples, respectively), combined pairwise and analyzed by LC-MS/MS. This protocol yielded high average sequence coverage (>40%) for the six tested proteins, and, when the datasets were searched for the predicted differential modification caused by fragment adduction with lysine residues, the site(s) of fragment reactivity could be directly identified. The fractional engagement of fragments at these sites was also determined by measuring the relative MS1 chromatographic peak intensities (R values) for the corresponding unmodified peptides derived from the DMSO and fragment-treated samples, respectively. For each of the representative proteins evaluated by this approach (PFKP, PNPO, HK1, HDHD3, XRCC6 and SIN3A), definitive evidence was obtained that the liganded lysine assigned by isoTOP-ABPP was directly adducted by the corresponding electrophilic fragment (FIG. 5B and Tables 1 and 3). In all cases, both the covalent peptide-fragment adducts were identified (FIG. 5B, insets, and Tables 1 and 3) and depletion of the unmodified tryptic peptide containing the liganded lysine and/or the adjacent peptide requiring the liganded lysine as a cleavage site (FIG. 5B, blue dots). Other tryptic peptides generated by a lysine cleavage event were unaffected by fragment electrophile treatment (FIG. 5B, black dots), indicating the specificity of fragment reactions with individual lysines on the tested proteins (as also predicted by isoTOP-ABPP; see FIG. 3D). These data indicate that the ligandability events assigned to lysines in human cell proteomes by isoTOP-ABPP correspond to direct, site-specific, and near-complete reactions with fragment electrophiles.

Functional Analysis of Fragment-Lysine Interactions

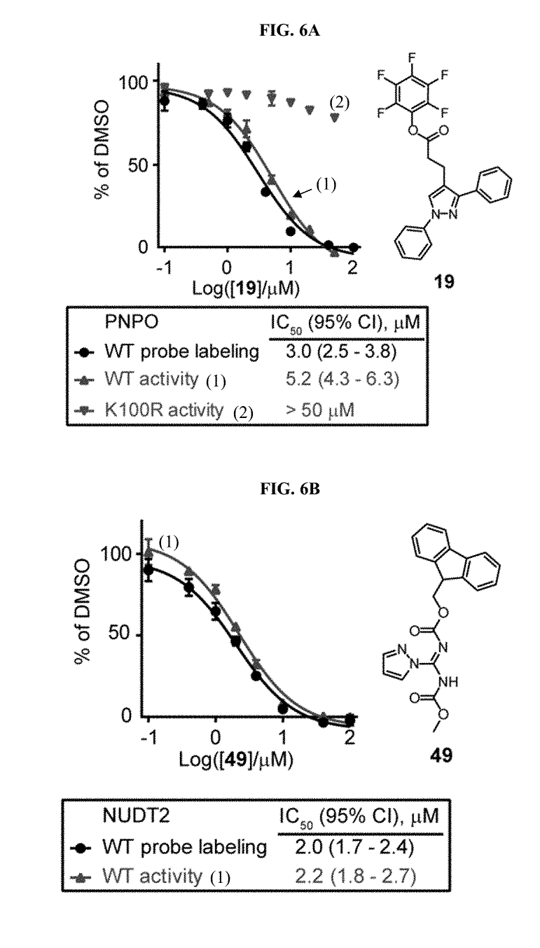

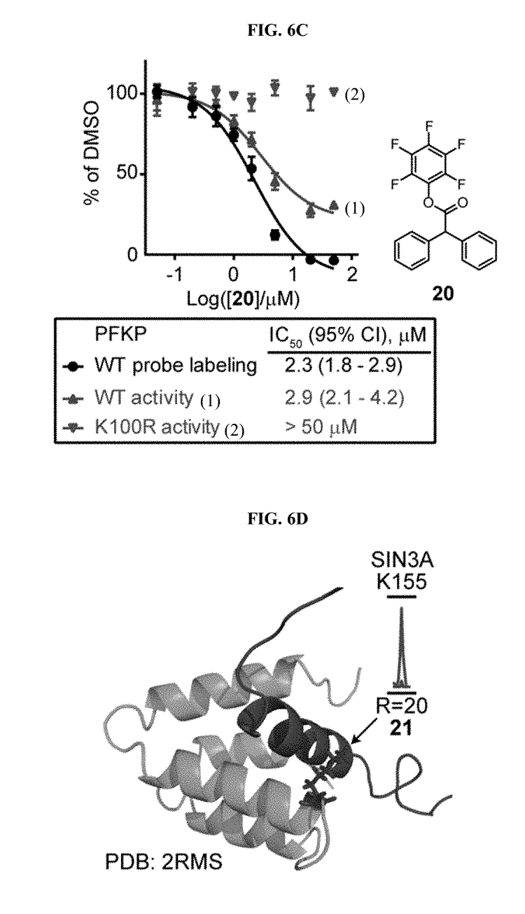

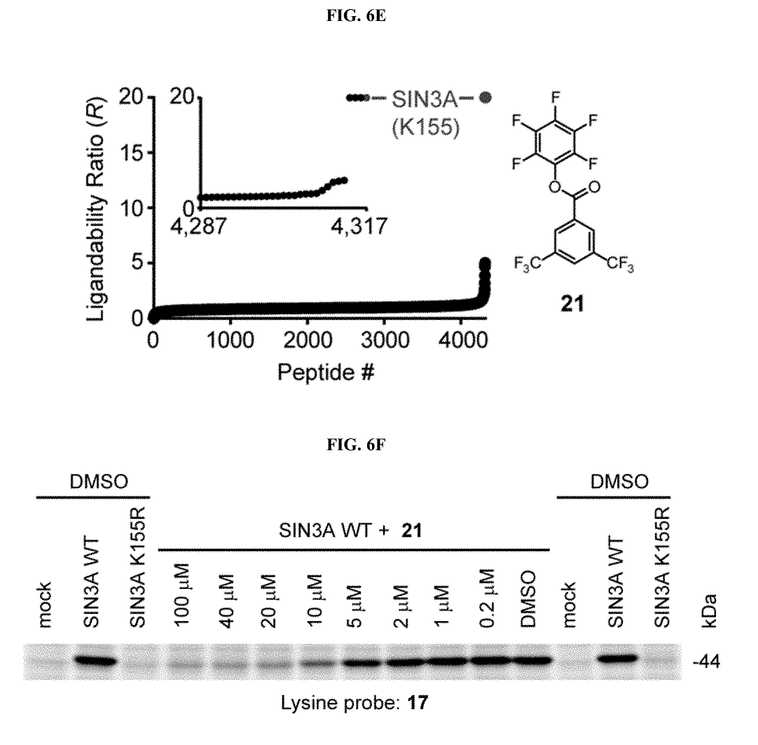

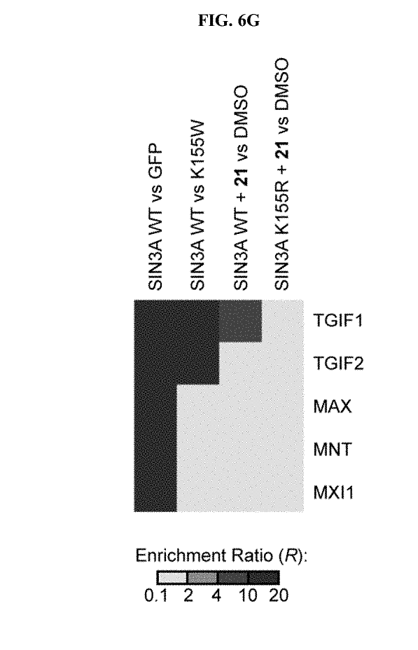

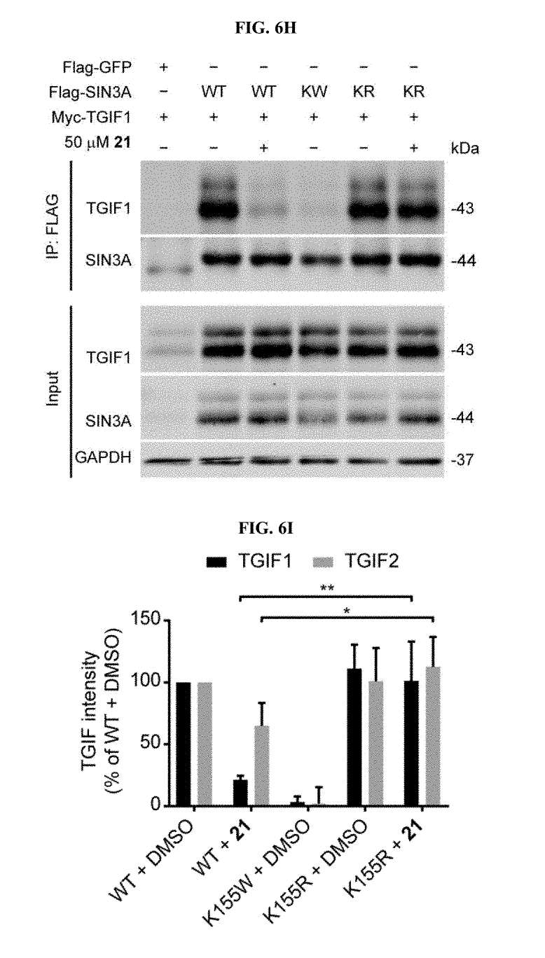

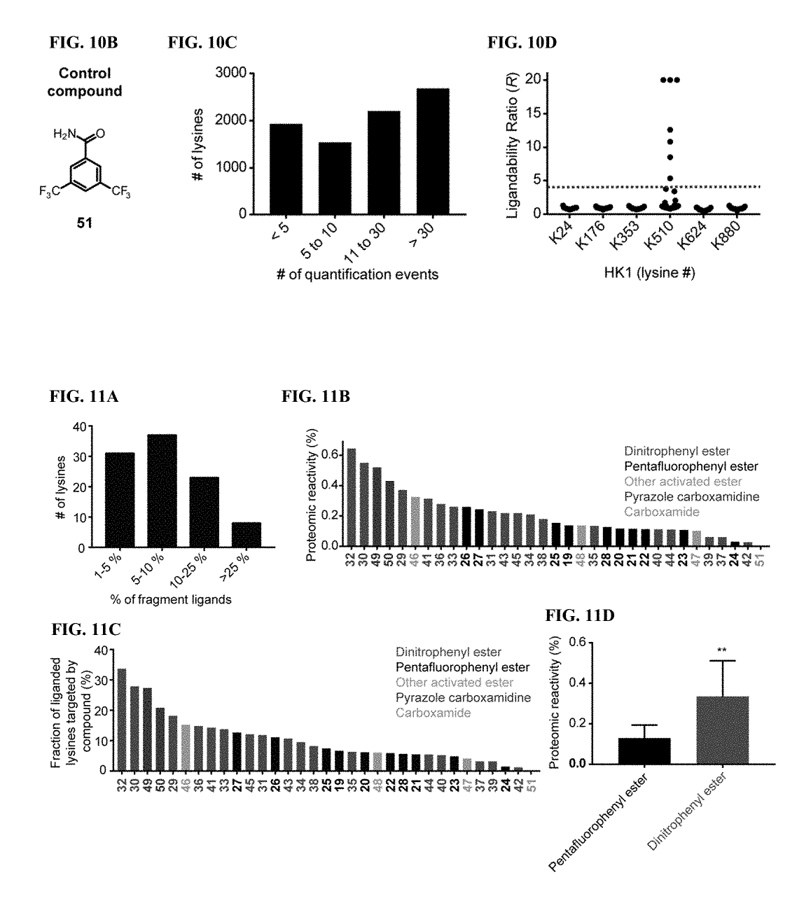

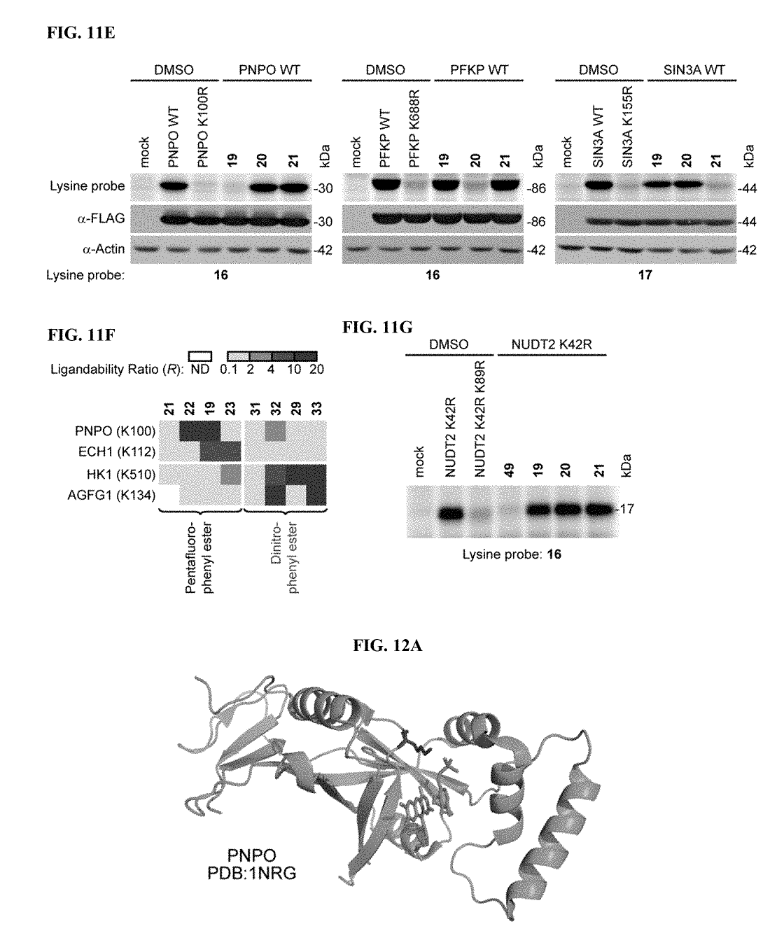

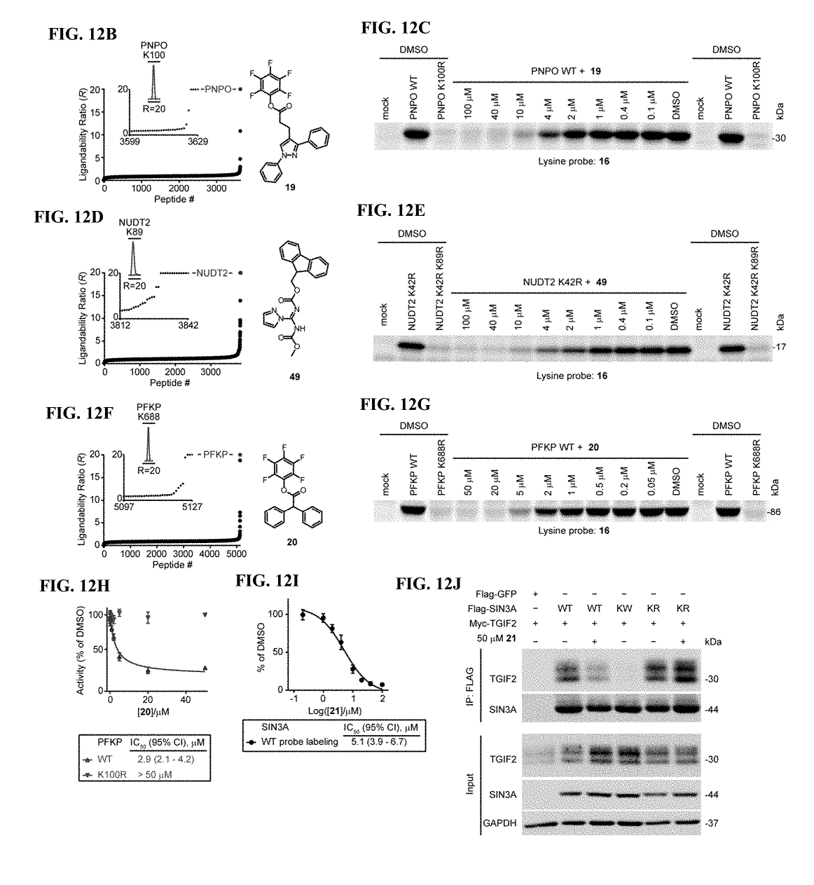

Next, the functional impact of fragment-lysine interactions mapped by isoTOP-ABPP was determine. As initial case studies, two enzymes with liganded active-site lysines-pyridoxamine-5′-phosphate oxidase (PNPO) and NUDT2 were selected. PNPO catalyzes the FMN-dependent oxidation of pyridoxamine-5′-phosphate and pyridoxine-5′-phosphate to pyridoxal-5′-phosphate in vitamin B₆ synthesis. PNPO possesses a hyper-reactive lysine K100 (R_{10:1}=0.7; Table 2) located in the enzyme's active site and shown in previous structural studies to interact with substrate (FIG. 12A). Competitive isoTOP-ABPP uncovered a highly restricted SAR for ligand engagement of K100, with only two fragments (19 and 22) fully blocking probe 1 labeling of this residue (FIG. 12B and Tables 1 and 3). It was confirmed by gel-based ABPP that fragment 19 blocked probe labeling of K100 in PNPO with an apparent IC₅₀ value of 3 μM (FIG. 6A and FIG. 12C). A similar IC₅₀ value (about 5 μM) was measured for blockade of PNPO catalytic activity by 19 using a substrate assay (FIG. 6A). The inhibitory effect of 19 was not observed with a K100R mutant of PNPO (FIG. 6A), which also did not label with amine-reactive probes (FIG. 12C).

NUDT2 is responsible for the catabolism of nucleotide cellular stress signals in human cells and was found to contain a hyper-reactive and liganded lysine K89 that is located proximal to the enzyme's nucleotide-binding site (FIG. 9E). K89 also exhibited a restricted SAR by isoTOP-ABPP, preferentially reacting with the two N,N′-diacyl-pyrazolecarboxamidine fragments 49 and 50 (FIG. 12D and Tables 1 and 3). It was confirmed by gel-based ABPP that fragment 49 blocked probe labeling of NUDT2 with an apparent IC₅₀ of 2 μM (FIG. 6B and FIG. 12E), and an equivalent IC₅₀ value was measured for inhibition of NUDT2 activity using a substrate assay (FIG. 6B). Since mutation of K89 to arginine (K89R) inactivated NUDT2 in the substrate assay (FIG. 2D), the inhibitory effect of 49 on the K89R mutant was not tested, but it was confirmed by gel-based ABPP that the K89R mutant showed a substantial reduction in amine-reactive probe labeling equivalent to that observed following treatment of NUDT2 with 49 (FIG. 12E).

Next, liganded lysines residing in more poorly characterized sites on proteins, specifically, a putative allosteric pocket in PFKP and a protein-protein interaction site in SIN3A were studied. PFKP is responsible for the phosphorylation of fructose-6-phosphate to fructose-1,6-bisphosphate, the committed step of glycolysis. Probe 1 labeling of the hyper-reactive lysine K688 in PFKP was completely blocked by fragment 20, which otherwise exhibited limited reactivity across the proteome (FIG. 4A and FIGS. 11B and 12F). Gel-based ABPP confirmed that 20 blocked probe labeling of recombinant PFKP with an apparent IC₅₀ of 2 μM (FIG. 6C and FIG. 12G), and a similar loss in probe reactivity was observed for the K688R mutant of PFKP (FIG. 12G). Using an enzyme-coupled assay monitoring the conversion of NAD⁺ to NADH by UV absorbance, it was found that the activity of WT-PFKP, but not the K688R-PFKP mutant was inhibited by 20 with an apparent IC₅₀ of 2.9 μM (FIG. 6C and FIG. 12H). Fragment 20 inhibition of the catalytic activity of WT-PFKP plateaued at about 80% reduction in substrate turnover (FIG. 6C and FIG. 12H), indicating that ligand reactivity at the K688 allosteric site substantially, but incompletely blocks enzyme function.

SIN3A is a multi-domain 145 kDa transcriptional repressor involved in histone deacetylase regulation and suppression of MYC-responsive genes. It was found that SIN3A contains a hyper-reactive lysine K155 (R_{10:1}=1.2; Table 2) located in the first paired amphipathic helix (PAH1) domain of the protein (FIG. 6D). Our isoTOP-ABPP experiments revealed that fragment 21 engages K155 in SIN3A (FIG. 6D, inset, and FIG. 6E), but otherwise shows low proteome-wide reactivity (FIG. 6E and FIG. 11B). A Flag-tagged SIN3A variant containing the N-terminal PAH1 and PAH2 protein-protein interaction domains (a.a. 1-400) was recombinantly expressed in HEK293T cells and found that treatment of cell lysates with 21 produced a site-specific and complete blockade of probe labeling of K155 with an apparent IC₅₀ of 5 μM (FIG. 6F and FIG. 12I). Quantitative SILAC (Stable Isotopic Labeling with Amino acids in Cell culture⁵⁸) proteomics was then used to identify SIN3A-interacting proteins that were sensitive to mutation of K155 and/or treatment with 21. HEK293T cells metabolically labeled with isotopically differentiated amino acids were transfected with cDNA constructs for Flag-SIN3A (heavy-labeled cells) or Flag-GFP (light-labeled cells), harvested, lysed, and immunoprecipitated with anti-Flag antibodies. Heavy and light-labeled immunoprecipitates were combined and subjected to tryptic digestion followed by LC-MS/MS analysis, which furnished a set of SIN3A-interacting proteins, defined as proteins that were substantially (>five-fold) enriched in the SIN3A-transfected compared to GFP-transfected samples (FIG. 6G and Tables 1 and 3). Similar quantitative proteomic experiments compared WT-SIN3A to a K155W-SIN3A mutant and DMSO-treated WT-SIN3A to 21-treated WT-SIN3A. The K155W mutant, which was generated to mimic incorporation of a bulky hydrophobic group into the 21-sensitive pocket of SIN3A, failed to substantially enrich two established SIN3-interacting proteins—TGIF1 and TGIF2^{59,60}—that co-immunoprecipitated with WT-SIN3A (FIG. 6G and Tables 1 and 3). Treatment with 21 also strongly blocked the TGIF1-SIN3A interaction, but only produced a marginal effect on TGIF2-SIN3A interaction (FIG. 6G and Tables 1 and 3). Other known SIN3A-interacting proteins that co-immunoprecipitated with WT-SIN3A, such as MAX, MNT and MXII, were not affected by K155W mutation or 21 treatment (FIG. 6G).

The effect of 21 on SIN3A interactions with TGIF1/TGIF2 by co-expressing these proteins with complementary epitope tags (Flag and Myc, respectively) was further evaluated. In this system, fragment 21 treatment, as well as K155W mutation, blocked the co-immunoprecipitation of TGIF1 as measured by anti-Myc blotting (FIG. 6H, I). The K155W mutant also strongly inhibited co-immunoprecipitation of TGIF2 with SIN3A, while 21 exerted a partial blockade of this association (FIG. 6I and FIG. 12J). Importantly, mutation of K155 to arginine (K155R) conferred resistance to the effects of 21 on the SIN3A-TGIF1 interaction (FIG. 6H, 6I and FIG. 12J). Taken together, these data demonstrate that covalent ligands targeting K155 in SIN3A might pharmacologically disrupt a select subset of protein-protein interactions implicated in gene regulation.

Table 1A-Table 1D illustrate a list of liganded lysines and their reactivity profiles with the fragment electrophile library from isoTOP-ABPP experiments performed in cell lysates (in vitro).

TABLE 1A

| Identifier | Protein Name and Illustrative Peptide Sequence | 19¹ | 20² | 21³ | 22⁴ | 23⁵ |
|---|---|---|---|---|---|---|
| A0AVT1_K409 | UBA6 Ubiquitin-like modifier-activating enzyme 6 | 1.5 | — | — | 1.6 | 1.3 |
| O14879_K148 | IFIT3 Interferon-induced protein with tetratricopeptide | 0.9 | 0.7 | 1.4 | — | — |
| O43399_K90 | TPD52L2 Tumor protein D54 | 2.6 | 1.5 | 2.0 | 3.6 | 1.5 |
| O43747_K214 | AP1G1 AP-1 complex subunit gamma-1 | 1.0 | 0.9 | 0.9 | 1.1 | 1.1 |
| O43837_K207 | IDH3B Isocitrate dehydrogenase | — | 6.5 | — | — | — |
| O60664_K140 | PLIN3 Perilipin-3 | 1.5 | — | — | — | — |
| O60664_K257 | PLIN3 Perilipin-3 | 2.2 | 2.5 | 2.0 | 2.6 | — |
| O75323_K75 | GBAS Protein NipSnap homolog 2 | 1.0 | 0.9 | 0.8 | 1.5 | 1.1 |
| O75821_K280 | EIF3G Eukaryotic translation initiation factor 3 subunit | — | 0.9 | 1.7 | 0.6 | — |
| O95197_K1022 | RTN3 Reticulon-3 | — | — | — | — | — |
| O95628_K134 | CNOT4 CCR4-NOT transcription complex subunit 4 | 1.1 | 0.9 | 1.2 | 1.4 | 1.2 |
| P00367_K480 | GLUD1 Glutamate dehydrogenase 1, mitochondrial | 1.0 | 1.4 | 1.2 | 2.5 | 1.8 |
| P02545_K135 | LMNA Prelamin-A/C | 1.1 | 1.0 | 1.0 | 1.0 | 0.9 |
| P02766_K35 | TTR Transthyretin | — | 1.4 | 2.6 | 2.6 | 1.0 |
| P04179_K68 | SOD2 Superoxide dismutase | — | 1.0 | 1.2 | 1.0 | — |
| P04181_K66 | OAT Ornithine aminotransferase, mitochondrial | — | — | — | — | — |
| P05141_K23 | SLC25A5 ADP/ATP translocase 2 | — | — | 1.7 | — | — |
| P07195_K244 | LDHB L-lactate dehydrogenase B chain | — | 1.0 | 1.0 | 1.0 | — |
| P07195_K82 | LDHB L-lactate dehydrogenase B chain | — | 1.2 | — | 0.8 | — |
| P07954_K311 | FH Fumarate hydratase, mitochondrial | — | 1.2 | 1.3 | — | 2.6 |
| P08237_K678 | PFKM 6-phosphofructokinase, muscle type | 1.0 | 1.4 | 0.9 | 1.1 | 1.0 |
| P0CG30_K53 | GSTT2B Glutathione S-transferase theta-2B | 1.0 | 1.0 | 0.9 | 1.0 | 0.9 |
| P11413_K171 | G6PD Glucose-6-phosphate 1-dehydrogenase | 0.9 | 0.7 | 0.8 | 0.7 | 2.1 |
| P11586_K760 | MTHFD1 C-1-tetrahydrofolate synthase, cytoplasmic | — | — | — | — | — |
| P12956_K351 | XRCC6 X-ray repair cross-complementing protein 6 | 1.7 | 2.3 | 1.4 | 3.6 | 1.2 |
| P13639_K235 | EEF2 Elongation factor 2 | 1.2 | 1.1 | 1.1 | 2.9 | 1.6 |
| P13639_K318 | EEF2 Elongation factor 2 | 1.1 | 1.1 | 1.2 | 5.6 | 1.6 |
| P13726_K279 | F3 Tissue factor | — | — | — | — | — |
| P13804_K139 | ETFA Electron transfer flavoprotein subunit alpha, | — | 0.9 | — | 0.9 | 1.4 |
| | mitochondrial | | | | | |
| P16930_K241 | FAH Fumarylacetoacetase | 2.5 | 1.0 | 2.0 | 3.0 | 2.8 |
| P17405_K118 | SMPD1 Sphingomyelin phosphodiesterase | — | 1.4 | — | — | — |
| P17858_K315 | PFKL 6-phosphofructokinase, liver type | 1.0 | — | — | — | — |
| P17858_K677 | PFKL 6-phosphofructokinase, liver type | 1.2 | 18.8 | 0.9 | 2.7 | 1.8 |
| P17858_K715 | PFKL 6-phosphofructokinase, liver type | 1.6 | 1.3 | 0.9 | 2.9 | 0.6 |
| P19367_K510 | HK1 Hexokinase-1 | 1.2 | 0.9 | 1.0 | 1.3 | 2.0 |
| P20248_K54 | CCNA2 Cyclin-A2 | — | — | — | — | — |
| P20839_K208 | IMPDH1 Inosine-5-monophosphate dehydrogenase 1 | — | — | — | — | — |
| P22830_K304 | FECH Ferrochelatase, mitochondrial | 1.8 | 1.7 | 1.0 | 2.0 | — |
| P23381_K256 | WARS Tryptophan--tRNA ligase, cytoplasmic | 1.2 | 1.3 | 1.1 | 1.0 | — |
| P23919_K118 | DTYMK Thymidylate kinase | — | 0.9 | 0.8 | — | — |
| P24941_K33 | CDK2 Cyclin-dependent kinase 2 | — | 0.9 | 1.0 | 1.5 | — |
| P26358_K45 | DNMT1 DNA (cytosine-5)-methyltransferase 1 | — | 7.3 | 1.4 | 0.9 | — |
| P26641_K227 | EEF1G Elongation factor 1-gamma | — | 0.9 | 0.8 | — | — |
| P27635_K121 | RPL10 60S ribosomal protein L10 | 0.9 | 0.7 | 0.8 | 0.9 | — |
| P32969_K82 | RPL9P9 60S ribosomal protein L9 | — | — | — | — | — |
| P36551_K404 | CPOX Coproporphyrinogen-III oxidase, mitochondrial | 10.8 | 1.1 | 1.0 | 1.1 | 1.3 |
| P42330_K270 | AKR1C3 Aldo-keto reductase family 1 member C3 | — | 1.4 | — | — | — |
| P42345_K2066 | MTOR Serine/threonine-protein kinase mTOR | 0.9 | 1.0 | 0.7 | 2.8 | 1.1 |
| P46734_K93 | MAP2K3 Dual specificity mitogen-activated protein | 0.9 | 0.9 | 0.9 | 0.9 | 0.9 |
| | kinase | | | | | |
| P46783_K139 | RPS10 40S ribosomal protein S10 | 1.0 | 0.6 | 0.5 | 0.9 | — |
| P50583_K89 | NUDT2 Bis(5-nucleosyl)-tetraphosphatase | 1.0 | 0.9 | 0.9 | 0.9 | 1.0 |
| P51580_K32 | TPMT Thiopurine S-methyltransferase | 1.3 | 0.9 | 4.9 | 1.3 | 6.4 |
| P52292_K459 | KPNA2 Importin subunit alpha-2 | 2.3 | 5.5 | 1.0 | 6.2 | 1.9 |
| P52594_K134 | AGFG1 Arf-GAP domain and FG repeat-containing | 1.0 | 1.0 | — | 1.4 | 1.7 |
| | protein 1 | | | | | |
| P52815_K87 | MRPL12 39S ribosomal protein L12, mitochondrial | — | 0.9 | 0.6 | 1.4 | — |
| P55263_K88 | ADK Adenosine kinase | 0.8 | 0.8 | 0.8 | 1.0 | 0.7 |
| P55786_K712 | NPEPPS Puromycin-sensitive aminopeptidase | 1.0 | 0.9 | 0.9 | 1.3 | 1.2 |
| P60520_K46 | GABARAPL2 Gamma-aminobutyric acid receptor- | 1.2 | 0.9 | 0.8 | 0.8 | — |
| | associated protei | | | | | |
| P61011_K81 | SRP54 Signal recognition particle 54 kDa protein | — | 1.0 | 0.7 | 0.9 | — |
| P61221_K191 | ABCE1 ATP-binding cassette sub-family E member 1 | 0.9 | 0.9 | 1.0 | 1.5 | 1.3 |
| P61221_K478 | ABCE1 ATP-binding cassette sub-family E member 1 | 1.1 | 1.2 | — | 1.6 | 1.5 |
| P61289_K12 | PSME3 Proteasome activator complex subunit 3 | 1.0 | 0.9 | 1.0 | 0.9 | — |
| P61289_K237 | PSME3 Proteasome activator complex subunit 3 | 0.2 | 0.9 | 0.9 | 1.1 | 0.5 |
| P61978_K405 | HNRNPK Heterogeneous nuclear ribonucleoprotein K | 1.3 | 1.0 | 1.0 | — | — |
| P62333_K72 | PSMC6 26S protease regulatory subunit 10B | — | — | — | — | — |
| P62875_K67 | POLR2L DNA-directed RNA polymerases I, II, and III | 1.0 | 0.9 | 0.8 | 0.9 | 1.1 |
| | subunit | | | | | |
| P68104_K84 | EEF1A1 Elongation factor 1-alpha 1 | — | — | — | — | — |
| Q00765_K147 | REEP5 Receptor expression-enhancing protein 5 | — | — | — | 4.8 | — |
| Q01813_K688 | PFKP 6-phosphofructokinase type C | 1.5 | 20.0 | 1.1 | 2.0 | 2.1 |
| Q0VFZ6_K312 | CCDC173 Coiled-coil domain-containing protein 173 | — | — | 20.0 | — | — |
| Q12931_K699 | TRAP1 Heat shock protein 75 kDa, mitochondrial | 1.2 | 1.0 | 0.9 | 1.1 | 1.0 |
| Q13011_K112 | ECH1 Delta(3,5)-Delta(2,4)-dienoyl-CoA isomerase, | 4.7 | 1.4 | 1.5 | 1.5 | 4.7 |
| | mitochondrial | | | | | |
| Q13033_K755 | STRN3 Striatin-3 | 0.9 | 0.9 | 0.9 | 1.0 | 1.2 |
| Q13148_K114 | TARDBP TAR DNA-binding protein 43 | — | — | 0.9 | 0.8 | — |
| Q13561_K175 | DCTN2 Dynactin subunit 2 | — | 1.0 | 0.9 | 1.7 | 1.1 |
| Q13617_K489 | CUL2 Cullin-2 | 0.8 | 0.8 | 0.9 | 1.0 | 0.9 |
| Q13630_K108 | TSTA3 GDP-L-fucose synthase | 0.8 | — | 1.0 | 1.1 | 1.0 |
| Q14204_K1649 | DYNC1H1 Cytoplasmic dynein 1 heavy chain 1 | 1.0 | 1.0 | 1.4 | 1.1 | 1.7 |
| Q14789_K103 | GOLGB1 Golgin subfamily B member 1 | 0.9 | 1.0 | 1.0 | 1.0 | 1.0 |
| Q14914_K194 | PTGR1 Prostaglandin reductase 1 | — | 1.6 | — | — | — |
| Q14CX7_K575 | NAA25 N-alpha-acetyltransferase 25, NatB auxiliary | 1.0 | 1.1 | 0.9 | 2.2 | — |
| | subunit | | | | | |
| Q15041_K188 | ARL6IP1 ADP-ribosylation factor-like protein 6- | — | 1.8 | 3.9 | — | — |
| | interacting | | | | | |
| Q15233_K467 | NONO Non-POU domain-containing octamer-binding | — | 1.2 | 1.5 | — | — |
| | protein | | | | | |
| Q16864_K6 | ATP6V1F V-type proton ATPase subunit F | — | 1.1 | 1.1 | 1.1 | — |
| Q2M389_K302 | KIAA1033 WASH complex subunit 7 | 0.8 | 0.9 | — | 3.6 | 1.5 |
| Q5TFE4_K123 | NT5DC1 5-nucleotidase domain-containing protein 1 | 20.0 | 1.0 | 1.2 | — | 1.5 |
| Q6NUQ1_K771 | RINT1 RAD50-interacting protein 1 | 1.2 | 1.0 | 2.2 | 1.0 | 1.1 |
| Q6NZI2_K326 | PTRF Polymerase I and transcript release factor | — | — | — | 20.0 | — |
| Q7L0Y3_K325 | TRMT10C Mitochondrial ribonuclease P protein 1 | — | 1.1 | 0.9 | 1.1 | 1.3 |
| Q8N163_K112 | KIAA1967 DBIRD complex subunit KIAA1967 | 1.2 | 1.0 | 1.0 | 1.1 | 1.1 |
| Q8N163_K97 | KIAA1967 DBIRD complex subunit KIAA1967 | 0.9 | 0.9 | 0.7 | 1.0 | 0.8 |
| Q8TCA0_K43 | LRRC20 Leucine-rich repeat-containing protein 20 | 1.1 | 0.7 | 1.0 | 0.9 | — |
| Q92600_K230 | RQCD1 Cell differentiation protein RCD1 homolog | 1.0 | 0.9 | 0.9 | 1.0 | 1.1 |
| Q969Y2_K492 | GTPBP3 tRNA modification GTPase GTPBP3, | — | 1.1 | 1.3 | 1.1 | — |
| | mitochondrial | | | | | |
| Q96AB3_K178 | ISOC2 Isochorismatase domain-containing protein 2, | 1.2 | 0.9 | 1.0 | 1.0 | 1.2 |
| | mitochondrial | | | | | |
| Q96C01_K99 | FAM136A Protein FAM136A | — | 0.9 | 1.1 | — | — |
| Q96EL2_K94 | MRPS24 28S ribosomal protein S24, mitochondrial | 1.4 | 0.9 | 0.8 | 1.6 | 2.1 |
| Q96HE7_K413 | ERO1L ERO1-like protein alpha | 0.5 | 0.9 | 1.4 | — | — |
| Q96ST3_K155 | SIN3A Paired amphipathic helix protein Sin3a | 1.6 | 1.2 | 20.0 | 3.9 | 4.1 |
| Q9BRQ3_K60 | NUDT22 Nucleoside diphosphate-linked moiety X motif | 0.9 | 0.8 | 0.8 | — | — |
| | 22 | | | | | |
| Q9BSH5_K15 | HDHD3 Haloacid dehalogenase-like hydrolase domain- | 20.0 | 1.1 | 1.0 | 1.1 | 1.3 |
| | containing protein 3 | | | | | |
| Q9BYT8_K253 | NLN Neurolysin, mitochondrial | 1.0 | 0.9 | 0.7 | 1.5 | — |
| Q9GZQ8_K51 | MAP1LC3B Microtubule-associated proteins 1A/1B | 1.0 | 0.9 | — | 1.2 | 0.7 |
| | light chain | | | | | |
| Q9GZV4_K47 | EIF5A2 Eukaryotic translation initiation factor 5A-2 | — | — | 5.0 | — | — |
| Q9H3P7_K117 | ACBD3 Golgi resident protein GCP60 | 1.0 | 1.2 | 0.9 | 1.2 | 1.2 |
| Q9H4M9_K511 | EHD1 EH domain-containing protein 1 | 0.8 | — | — | — | — |
| Q9H6D7_K271 | HAUS4 HAUS augmin-like complex subunit 4 | — | 0.7 | — | 0.9 | — |
| Q9H9B4_K170 | SFXN1 Sideroflexin-1 | — | — | — | — | — |
| Q9HC38_K305 | GLOD4 Glyoxalase domain-containing protein 4 | 0.9 | 0.8 | 0.8 | 0.7 | 0.9 |
| Q9NQC3_K1104 | RTN4 Reticulon-4 | — | 2.4 | 2.5 | — | 3.3 |
| Q9NTK5_K248 | OLA1 Obg-like ATPase 1 | 1.0 | 0.9 | 1.0 | 1.0 | 1.0 |
| Q9NUJ1_K185 | ABHD10 Abhydrolase domain-containing protein 10, | 1.2 | 1.1 | 4.7 | 1.5 | 2.0 |
| | mitochondrial | | | | | |
| Q9NVS9_K100 | PNPO Pyridoxine-5-phosphate oxidase | 20.0 | 0.9 | 0.9 | 20.0 | 1.8 |
| Q9NZ08_K685 | ERAP1 Endoplasmic reticulum aminopeptidase 1 | 1.1 | 1.5 | 1.4 | 2.2 | 1.7 |
| Q9UBP0_K180 | SPAST Spastin | 1.3 | 1.2 | 1.8 | 1.0 | — |
| Q9UBT2_K409 | UBA2 SUMO-activating enzyme subunit 2 | — | 4.1 | — | — | — |
| Q9UHY7_K111 | ENOPH1 Enolase-phosphatase E1 | 1.0 | 1.0 | 1.0 | 1.4 | 1.3 |
| Q9UKV3_K103 | ACIN1 Apoptotic chromatin condensation inducer in the | — | — | — | — | — |
| | nucleus | | | | | |
| Q9Y4K4_K49 | MAP4K5 Mitogen-activated protein kinase kinase kinase | — | — | 0.6 | — | — |
| | kinase 5 | | | | | |
| Q9Y5K5_K323 | UCHL5 Ubiquitin carboxyl-terminal hydrolase isozyme | 1.1 | 0.9 | 1.0 | 0.8 | — |
| | L5 | | | | | |
| Q9Y5X2_K316 | SNX8 Sorting nexin-8 | — | — | — | — | — |

19¹ - 50 uM_231_sol_invitro
20² - 50 uM_invitro_sol_231
21³ - 50 uM_invitro_sol_231
22⁴ - 50 uM_invitro_sol_231
23⁵ - 50 uM_231_sol_invitro

TABLE 1B

| Identifier | 24¹ | 25² | 26³ | 27⁴ | 28⁵ | 29⁶ | 30⁷ | 31⁸ | 32⁹ | 33¹⁰ |
|---|---|---|---|---|---|---|---|---|---|---|
| A0AVT1_K409 | — | 1.4 | — | — | — | — | — | — | — | — |
| O14879_K148 | 1.3 | 1.9 | 1.4 | 0.9 | 1.0 | 4.1 | — | — | — | — |
| O43399_K90 | 1.9 | 1.5 | 1.3 | 1.4 | 1.1 | 3.1 | 5.5 | 3.3 | 2.7 | 1.3 |
| O43747_K214 | 1.0 | 1.0 | 1.1 | 0.9 | 1.0 | 1.0 | 1.1 | 0.9 | 6.6 | 1.7 |
| O43837_K207 | — | — | — | — | — | — | — | — | — | — |
| O60664_K140 | 1.7 | — | 1.2 | 1.9 | 1.2 | 2.1 | 4.5 | — | — | 1.1 |
| O60664_K257 | — | — | 4.1 | 1.8 | 1.1 | 3.5 | 2.9 | — | — | 2.2 |
| O75323_K75 | 1.0 | 1.4 | 4.2 | 20.0 | 1.0 | 1.0 | 1.1 | 0.8 | — | 3.0 |
| O75821_K280 | — | — | — | — | — | 5.3 | 1.0 | — | — | — |
| O95197_K1022 | — | — | — | — | 1.2 | 6.2 | 20.0 | — | — | 1.4 |
| O95628_K134 | 1.0 | 1.8 | 1.1 | 1.2 | 1.0 | 1.1 | 1.2 | 4.3 | 2.1 | 1.4 |
| P00367_K480 | 2.0 | 1.2 | 1.6 | 2.0 | 1.0 | 1.9 | 1.8 | 1.3 | 4.4 | 6.2 |
| P02545_K135 | — | 0.9 | — | — | — | 1.0 | 20.0 | 1.3 | 20.0 | 0.7 |
| P02766_K35 | — | — | — | — | — | 1.6 | 1.5 | 16.1 | 3.1 | 2.7 |
| P04179_K68 | — | — | 1.2 | 0.9 | 0.8 | — | 1.1 | 1.5 | 1.1 | — |
| P04181_K66 | 0.6 | — | — | — | — | — | — | — | 9.3 | 1.0 |
| P05141_K23 | — | — | — | — | 1.0 | 1.5 | 2.2 | 1.5 | 1.8 | — |
| P07195_K244 | — | — | — | — | — | — | 1.2 | — | 1.0 | — |
| P07195_K82 | — | — | 0.7 | — | — | — | 20.0 | — | 0.8 | — |
| P07954_K311 | — | — | 2.4 | — | — | — | 5.2 | 1.4 | — | 2.2 |
| P08237_K678 | 1.1 | 1.3 | 1.4 | 2.6 | 1.3 | 1.8 | 20.0 | 1.1 | 15.4 | 4.0 |
| P0CG30_K53 | 1.0 | 1.0 | 1.3 | 0.9 | 1.0 | 0.7 | 0.6 | 0.8 | 0.8 | 0.5 |
| P11413_K171 | 0.9 | 0.8 | 1.0 | 0.9 | 0.9 | 0.9 | 3.9 | 1.1 | 0.9 | 16.3 |
| P11586_K760 | — | — | — | — | — | — | 0.9 | 0.8 | 0.6 | — |
| P12956_K351 | 1.3 | 1.6 | 2.1 | 3.1 | 1.9 | 3.2 | 20.0 | 2.6 | 13.4 | 2.5 |
| P13639_K235 | 1.1 | 1.9 | 1.3 | 5.7 | 1.4 | 1.0 | 1.4 | 1.1 | 1.4 | 1.0 |
| P13639_K318 | 1.2 | 2.3 | 1.1 | — | 1.6 | 0.9 | 1.4 | 1.2 | 1.5 | 0.9 |
| P13726_K279 | — | — | — | — | — | — | — | — | — | — |
| P13804_K139 | 1.1 | — | — | — | — | 0.8 | 20.0 | 0.8 | 20.0 | 0.7 |
| P16930_K241 | 1.1 | 5.2 | 5.0 | 1.8 | 2.5 | 1.7 | 2.7 | 20.0 | 2.2 | 1.7 |
| P17405_K118 | — | — | — | — | — | 2.5 | — | — | — | — |
| P17858_K315 | 0.9 | 0.9 | — | — | — | 0.8 | 4.9 | — | 20.0 | 0.8 |
| P17858_K677 | 3.3 | 1.6 | 20.0 | 18.1 | 1.1 | 2.0 | 20.0 | 2.5 | 11.9 | 2.0 |
| P17858_K715 | 0.9 | 1.3 | 1.5 | 1.3 | 1.5 | 6.8 | — | 2.3 | 7.7 | 1.1 |
| P19367_K510 | — | 1.2 | 3.4 | 1.3 | 0.9 | 10.8 | 1.0 | 1.1 | 8.6 | 20.0 |
| P20248_K54 | — | — | — | — | — | — | — | — | — | 5.4 |
| P20839_K208 | — | — | — | — | — | 1.3 | 1.6 | — | — | 1.2 |
| P22830_K304 | 1.5 | 1.0 | 1.7 | 1.5 | 1.5 | 5.4 | — | 3.2 | — | 1.7 |
| P23381_K256 | — | 4.4 | 2.3 | 2.7 | 1.0 | 0.9 | 1.1 | 0.9 | 0.9 | 0.8 |
| P23919_K118 | 1.1 | 1.1 | — | — | 0.9 | 1.1 | — | — | — | 6.6 |
| P24941_K33 | — | — | — | — | — | 0.9 | — | — | — | 0.7 |
| P26358_K45 | — | 0.9 | — | — | — | 0.9 | — | — | 1.0 | 0.9 |
| P26641_K227 | 0.9 | 1.1 | 0.8 | 1.2 | 15.0 | 0.7 | 0.9 | 2.2 | — | 0.7 |
| P27635_K121 | — | — | 0.9 | — | — | 1.4 | 1.1 | — | 1.7 | — |
| P32969_K82 | — | — | — | — | — | — | — | 0.9 | 1.0 | — |
| P36551_K404 | 1.2 | 1.3 | 1.1 | 1.2 | 0.9 | 18.1 | 1.1 | 1.1 | 1.0 | 1.0 |
| P42330_K270 | 4.2 | — | — | 2.7 | 1.8 | 2.1 | — | — | — | 1.0 |
| P42345_K2066 | 1.0 | — | 2.8 | 2.5 | 1.0 | 1.0 | 3.5 | 1.1 | 8.0 | 1.1 |
| P46734_K93 | — | — | 1.1 | — | — | 0.9 | 1.4 | 1.2 | 1.4 | 0.9 |
| P46783_K139 | 1.2 | — | 1.0 | — | — | 20.0 | 1.2 | — | — | 0.7 |
| P50583_K89 | 0.9 | 1.0 | 0.9 | 0.7 | 1.0 | 1.0 | 1.1 | 0.8 | 3.7 | 1.9 |
| P51580_K32 | 1.0 | 2.9 | 1.0 | 1.3 | 1.1 | 1.4 | 1.5 | 2.5 | 2.5 | 3.7 |
| P52292_K459 | 1.2 | 1.3 | 8.2 | 1.3 | 1.3 | 2.0 | 4.3 | 1.3 | 1.8 | 1.8 |
| P52594_K134 | 1.5 | 1.1 | 2.2 | 1.5 | 1.0 | 1.4 | 14.3 | 1.2 | 19.9 | 20.0 |
| P52815_K87 | — | — | — | — | 0.8 | 1.1 | — | — | — | 2.1 |
| P55263_K88 | 1.0 | 0.8 | 0.9 | 0.8 | 0.8 | 1.0 | 0.8 | 0.7 | 1.4 | 0.9 |
| P55786_K712 | 1.0 | 1.1 | 1.2 | 1.2 | 1.0 | 1.1 | 1.5 | 1.1 | 1.4 | 1.2 |
| P60520_K46 | — | 1.2 | — | — | — | 0.9 | — | 1.2 | 1.1 | 1.2 |
| P61011_K81 | — | 0.8 | 1.0 | — | 0.8 | 1.1 | 20.0 | 0.9 | 20.0 | 1.3 |
| P61221_K191 | — | 1.7 | 1.4 | — | — | 1.0 | 1.1 | 1.4 | 1.3 | 1.1 |
| P61221_K478 | 1.4 | 1.5 | 1.3 | 2.1 | 1.0 | 1.2 | 1.1 | 1.1 | 1.2 | 1.2 |
| P61289_K12 | — | 0.9 | 0.9 | — | 1.0 | 1.1 | — | 1.1 | 4.7 | 2.1 |
| P61289_K237 | 0.2 | 0.5 | 1.1 | 0.8 | 0.6 | 0.4 | 4.3 | 1.1 | 5.1 | 1.1 |
| P61978_K405 | — | 1.3 | 0.9 | 1.0 | — | 20.0 | 0.9 | — | 0.9 | — |
| P62333_K72 | — | — | — | — | 20.0 | 20.0 | — | — | 1.4 | — |
| P62875_K67 | 0.9 | 1.0 | 1.1 | 1.0 | 1.0 | — | 1.8 | 0.9 | — | — |
| P68104_K84 | — | — | — | — | — | 20.0 | 0.5 | 0.4 | 0.4 | — |
| Q00765_K147 | — | — | — | — | — | — | 20.0 | 3.0 | — | — |
| Q01813_K688 | 2.5 | 15.6 | 6.4 | 20.0 | 1.3 | 12.8 | 20.0 | 2.3 | 12.6 | 20.0 |
| Q0VFZ6_K312 | — | — | — | — | — | — | — | — | — | — |
| Q12931_K699 | 0.9 | 0.9 | — | 0.7 | 0.9 | 2.2 | 4.1 | 1.0 | 2.0 | 1.5 |
| Q13011_K112 | — | 2.7 | — | 2.7 | 1.8 | 1.0 | 0.9 | 1.2 | 1.0 | 0.9 |
| Q13033_K755 | 1.1 | 1.0 | 1.0 | 1.0 | 0.9 | 1.1 | 2.0 | 1.4 | 2.7 | 1.8 |
| Q13148_K114 | 1.0 | — | — | — | — | 1.1 | — | — | — | — |
| Q13561_K175 | 1.3 | 1.1 | 1.6 | 1.2 | — | 1.3 | 3.4 | 1.0 | 4.4 | 1.6 |
| Q13617_K489 | 0.9 | 1.1 | 0.9 | 1.1 | 1.0 | 1.2 | 1.1 | 1.1 | 1.1 | 1.2 |
| Q13630_K108 | 0.6 | — | 1.4 | 4.6 | 1.0 | 1.1 | — | — | — | 1.2 |
| Q14204_K1649 | 1.3 | 1.7 | — | 4.2 | 1.0 | 1.1 | — | — | 1.2 | 1.1 |
| Q14789_K103 | 1.0 | 1.0 | 1.1 | 1.3 | 0.9 | 1.1 | 1.4 | 0.9 | 7.0 | 1.0 |
| Q14914_K194 | — | — | — | — | — | — | — | — | — | — |
| Q14CX7_K575 | 1.1 | 0.9 | 0.9 | 1.4 | 0.8 | 1.1 | 4.7 | 1.2 | 2.4 | 1.1 |
| Q15041_K188 | 2.4 | — | — | — | — | — | — | — | 5.9 | 1.3 |
| Q15233_K467 | 1.0 | — | — | — | — | — | — | 1.3 | 1.2 | 3.7 |
| Q16864_K6 | — | — | — | — | — | 0.8 | 20.0 | — | — | 0.8 |
| Q2M389_K302 | 1.0 | — | 1.8 | — | — | 1.1 | 1.6 | — | — | 0.9 |
| Q5TFE4_K123 | 1.1 | — | — | — | 1.2 | 1.2 | — | — | — | — |
| Q6NUQ1_K771 | 1.0 | 1.4 | 0.9 | 1.1 | 0.8 | 1.2 | 1.7 | 20.0 | 1.2 | 1.0 |
| Q6NZI2_K326 | — | — | — | — | — | 20.0 | — | — | — | — |
| Q7L0Y3_K325 | 1.4 | 1.1 | — | — | — | 0.7 | 1.1 | 1.1 | 1.0 | 0.9 |
| Q8N163_K112 | 1.0 | 1.1 | 1.1 | 1.2 | 1.1 | 1.2 | 1.4 | 1.0 | 1.2 | 0.9 |
| Q8N163_K97 | 0.9 | 0.7 | 0.9 | 0.9 | 0.9 | 0.8 | 1.0 | 0.9 | 1.1 | 0.7 |
| Q8TCA0_K43 | — | — | 0.8 | — | — | 1.3 | 1.5 | — | 1.6 | — |
| Q92600_K230 | — | — | 1.2 | — | — | 1.0 | 1.6 | 1.0 | 10.2 | 2.5 |
| Q969Y2_K492 | — | — | 5.0 | 1.6 | — | 20.0 | 1.5 | 1.4 | 1.8 | — |
| Q96AB3_K178 | — | 1.4 | — | 1.3 | 1.8 | 1.7 | 1.0 | 2.1 | 1.1 | 9.1 |
| Q96C01_K99 | — | — | 0.9 | — | — | 0.8 | 0.8 | — | 0.9 | 0.7 |
| Q96EL2_K94 | 1.5 | 1.3 | 2.7 | 2.0 | 1.0 | 2.2 | — | 1.6 | 5.1 | 1.4 |
| Q96HE7_K413 | — | — | — | — | 1.4 | 20.0 | 1.2 | — | 20.0 | — |
| Q96ST3_K155 | 1.1 | 11.1 | 1.7 | 11.7 | 4.9 | 1.5 | 20.0 | 18.6 | 17.5 | 2.0 |
| Q9BRQ3_K60 | — | 0.8 | 0.8 | 1.2 | 1.0 | 0.8 | — | 1.1 | 20.0 | — |
| Q9BSH5_K15 | 2.3 | 4.0 | 3.4 | 2.3 | 13.8 | 20.0 | 20.0 | 4.6 | 6.7 | 1.5 |
| Q9BYT8_K253 | — | 0.9 | 1.2 | — | — | 1.0 | 1.3 | 1.5 | 1.7 | 4.1 |
| Q9GZQ8_K51 | 1.1 | 0.9 | 1.2 | 1.0 | 1.0 | 0.8 | 1.2 | 1.1 | 0.9 | 0.8 |
| Q9GZV4_K47 | — | — | — | — | — | — | — | — | — | — |
| Q9H3P7_K117 | 1.0 | 1.7 | 1.8 | 1.9 | 1.1 | 1.0 | 1.7 | 1.1 | 1.3 | 1.1 |
| Q9H4M9_K511 | — | — | — | — | — | — | 0.9 | — | 1.2 | — |
| Q9H6D7_K271 | 0.8 | — | — | — | — | — | 1.0 | — | 1.0 | — |
| Q9H9B4_K170 | — | — | — | — | — | 20.0 | 4.5 | — | 8.6 | — |
| Q9HC38_K305 | 1.5 | 0.4 | 0.8 | 0.5 | 1.0 | 0.9 | 0.6 | 0.5 | — | 0.4 |
| Q9NQC3_K1104 | 2.3 | — | 5.3 | — | 1.0 | 15.6 | — | 6.1 | 20.0 | 4.3 |
| Q9NTK5_K248 | 1.1 | 1.0 | 1.0 | 1.1 | 1.0 | 0.9 | 1.0 | 1.0 | 1.0 | 1.0 |
| Q9NUJ1_K185 | — | 1.5 | 1.5 | 1.9 | 1.2 | 1.3 | 2.4 | 4.4 | 1.4 | 2.1 |
| Q9NVS9_K100 | 0.9 | 1.0 | 1.4 | 1.0 | 0.9 | 1.9 | 1.0 | 1.0 | 3.6 | 1.2 |
| Q9NZ08_K685 | 1.6 | 1.9 | 1.2 | 5.7 | 1.4 | 1.0 | 1.2 | 1.6 | 2.8 | 1.0 |
| Q9UBP0_K180 | 1.1 | 1.2 | — | 2.1 | 1.0 | 1.2 | — | 1.1 | 2.5 | 0.7 |
| Q9UBT2_K409 | — | — | — | — | — | — | 7.3 | 6.2 | — | — |
| Q9UHY7_K111 | 1.4 | 1.1 | 1.3 | 1.5 | 1.0 | 0.8 | 0.8 | — | 1.3 | 0.9 |
| Q9UKV3_K103 | — | — | — | — | — | — | — | — | — | — |
| Q9Y4K4_K49 | — | — | — | — | — | 1.8 | — | — | 5.3 | — |
| Q9Y5K5_K323 | 0.8 | — | 1.1 | 1.1 | 1.2 | 1.1 | 1.1 | 1.0 | 1.0 | 0.8 |
| Q9Y5X2_K316 | — | — | — | — | — | 3.4 | — | — | — | 4.1 |

24¹- 50 uM_231_sol_invitro
25²- 50 uM_231_sol_invitro
26³- 50 uM_231_sol_invitro
27⁴- 50 uM_231_sol_invitro
28⁵- 50 uM_231_sol_invitro
29⁶- 50 uM_231_sol_invitro
30⁷- 50 uM_in vitro_sol_231
31⁸- 50 uM_in vitro_sol_231
32⁹- 50 uM_in vitro_sol_231
33¹⁰- 50 uM_231_sol_invitro

TABLE 1C

| Identifier | 34¹ | 35² | 36³ | 37⁴ | 38⁵ | 39⁶ | 40⁷ | 41⁸ | 42⁹ | 43¹⁰ |
|---|---|---|---|---|---|---|---|---|---|---|
| A0AVT1_K409 | — | — | — | — | — | — | — | 6.6 | — | — |
| O14879_K148 | — | — | 3.4 | — | — | 1.4 | — | — | 0.8 | — |
| O43399_K90 | 1.8 | 1.3 | 1.3 | 1.6 | 2.9 | 1.3 | 1.2 | 1.3 | 1.0 | 1.1 |
| O43747_K214 | 1.7 | 1.0 | 1.1 | 0.9 | 0.9 | 0.8 | 0.9 | 1.0 | 0.8 | 1.1 |
| O43837_K207 | — | — | — | — | — | — | — | — | — | — |
| O60664_K140 | — | 1.1 | 1.5 | — | — | 2.0 | — | 2.1 | 0.9 | — |
| O60664_K257 | — | 1.2 | 3.9 | 3.5 | — | 2.2 | 1.6 | — | 0.9 | — |
| O75323_K75 | — | 1.6 | 20.0 | 1.6 | 1.3 | 20.0 | 1.0 | 1.8 | 0.9 | 1.6 |
| O75821_K280 | — | 1.0 | — | — | — | — | — | — | — | — |
| O95197_K1022 | — | — | — | — | — | — | 1.7 | 1.6 | 1.1 | — |
| O95628_K134 | 1.1 | 1.3 | — | 1.0 | 1.2 | 1.0 | 1.1 | 1.3 | 1.0 | 1.3 |
| P00367_K480 | 1.3 | 1.8 | 3.2 | 1.1 | 1.3 | 1.0 | 1.2 | 3.7 | 1.1 | 3.9 |
| P02545_K135 | 1.2 | 0.7 | 0.8 | 0.9 | 0.8 | 1.0 | 1.2 | 1.1 | 1.0 | 20.0 |
| P02766_K35 | 2.2 | 15.6 | 2.4 | — | 1.2 | 2.2 | — | 3.4 | 1.0 | — |
| P04179_K68 | — | — | 2.1 | 1.7 | — | 1.0 | — | 4.8 | — | 1.4 |
| P04181_K66 | — | — | — | — | — | — | — | — | — | — |
| P05141_K23 | 1.1 | 1.1 | 1.1 | 1.2 | 1.1 | 0.9 | 0.8 | 1.0 | 1.1 | 0.9 |
| P07195_K244 | 1.0 | — | 20.0 | 0.8 | — | 1.0 | — | 20.0 | — | — |
| P07195_K82 | — | 0.6 | 0.9 | 0.8 | 0.7 | 20.0 | 20.0 | — | 20.0 | — |
| P07954_K311 | — | — | — | 1.2 | — | 0.9 | — | 1.0 | — | — |
| P08237_K678 | 1.1 | 1.2 | 5.3 | 1.0 | 1.0 | 1.0 | 0.9 | 2.0 | 0.9 | 1.3 |
| P0CG30_K53 | 0.5 | 4.1 | 0.5 | 0.7 | 0.8 | 0.7 | 0.6 | 0.5 | 1.0 | 0.6 |
| P11413_K171 | 0.9 | 1.7 | 1.0 | 0.9 | 0.9 | 1.1 | 1.9 | 1.0 | 0.9 | 1.0 |
| P11586_K760 | — | — | — | — | 1.3 | — | — | 4.2 | — | — |
| P12956_K351 | 1.6 | 1.5 | 2.1 | 1.3 | 3.3 | 1.6 | 1.0 | 1.6 | 1.0 | 1.5 |
| P13639_K235 | 1.0 | 1.1 | 1.0 | 1.0 | 0.9 | 0.9 | 0.9 | 1.0 | 1.0 | 1.1 |
| P13639_K318 | — | 1.1 | 1.0 | 1.0 | 0.9 | 0.9 | 0.9 | 1.1 | 1.0 | 1.2 |
| P13726_K279 | — | — | — | — | — | 1.8 | — | — | — | 1.1 |
| P13804_K139 | — | 0.9 | 0.7 | 0.9 | — | 0.6 | 0.9 | 20.0 | — | — |
| P16930_K241 | 1.2 | 3.4 | 3.6 | 1.0 | 1.0 | 1.2 | 1.4 | 1.4 | 0.9 | 1.4 |
| P17405_K118 | — | 1.4 | — | — | 1.2 | 0.8 | — | 1.5 | — | — |
| P17858_K315 | — | — | — | 1.3 | 0.4 | — | — | 2.3 | 0.7 | — |
| P17858_K677 | 2.8 | 1.5 | 20.0 | 1.1 | 1.3 | 1.3 | 0.9 | 5.6 | 0.8 | 3.2 |
| P17858_K715 | 1.3 | 1.3 | 20.0 | 1.1 | 20.0 | 0.6 | 1.1 | 2.0 | 0.9 | 1.3 |
| P19367_K510 | 20.0 | 1.1 | 3.7 | 0.9 | 1.7 | 0.9 | — | 12.6 | 0.8 | 20.0 |
| P20248_K54 | — | — | — | 0.5 | — | — | — | — | — | — |
| P20839_K208 | — | — | 1.6 | 1.4 | 1.2 | — | — | 1.2 | 1.2 | 1.3 |
| P22830_K304 | — | — | 2.0 | — | 1.8 | 1.3 | — | — | 1.0 | 1.3 |
| P23381_K256 | 1.0 | 1.1 | 1.0 | 0.4 | 1.4 | 0.9 | 1.1 | 0.9 | 1.1 | 1.0 |
| P23919_K118 | — | 0.9 | — | 0.9 | — | 0.8 | — | 0.8 | — | — |
| P24941_K33 | 1.0 | 1.6 | — | 0.9 | 0.9 | 1.0 | 1.0 | 1.0 | 0.9 | — |
| P26358_K45 | — | — | 0.8 | 0.7 | — | 1.0 | — | — | — | — |
| P26641_K227 | — | — | — | — | — | — | — | 0.8 | 0.7 | 2.3 |
| P27635_K121 | 1.3 | 0.6 | 0.5 | 0.8 | 20.0 | 1.0 | — | 0.8 | — | 20.0 |
| P32969_K82 | — | 0.8 | — | — | — | 0.7 | — | 0.9 | — | 0.9 |
| P36551_K404 | 1.1 | 1.2 | 1.0 | 1.0 | 1.0 | 1.0 | 1.8 | 1.0 | 1.0 | 1.1 |
| P42330_K270 | 1.1 | 0.6 | 0.6 | 0.8 | 0.9 | 0.8 | 2.2 | 0.7 | 0.9 | — |
| P42345_K2066 | 1.1 | 1.1 | 3.0 | 1.0 | 1.1 | — | 0.8 | — | — | 1.6 |
| P46734_K93 | 0.8 | 1.0 | 0.9 | 0.7 | 0.9 | 0.8 | 1.0 | 0.9 | 0.9 | 1.0 |
| P46783_K139 | — | — | 0.7 | 0.6 | 1.0 | — | — | — | 1.0 | — |
| P50583_K89 | 1.0 | 1.6 | 0.9 | 1.4 | 0.9 | 2.4 | 0.9 | 1.0 | 1.0 | 1.1 |
| P51580_K32 | 1.6 | — | 3.4 | 1.5 | 1.1 | 1.0 | 1.1 | 1.5 | 0.9 | 1.5 |
| P52292_K459 | 1.0 | 1.3 | 9.2 | 0.9 | 20.0 | 1.0 | 1.7 | 2.3 | 0.9 | 2.6 |
| P52594_K134 | 20.0 | — | 20.0 | 1.1 | 1.4 | 1.0 | 0.8 | 20.0 | 1.0 | 4.8 |
| P52815_K87 | — | 0.9 | — | — | 0.9 | 1.1 | — | 4.1 | 1.0 | — |
| P55263_K88 | 20.0 | 0.6 | 20.0 | 0.8 | 0.9 | 0.9 | 0.9 | 1.5 | 1.0 | 0.9 |
| P55786_K712 | 11.0 | 1.3 | 2.7 | 0.9 | 1.0 | 0.9 | 1.0 | 1.5 | 0.9 | 2.3 |
| P60520_K46 | — | 0.9 | — | 0.8 | 1.0 | 0.8 | — | 1.0 | 0.9 | — |
| P61011_K81 | 0.8 | 0.8 | 1.3 | 0.9 | — | 0.9 | — | 1.0 | 0.9 | 20.0 |
| P61221_K191 | 1.0 | 1.1 | 1.0 | 0.9 | — | 0.8 | 0.9 | 1.2 | 1.0 | 1.3 |
| P61221_K478 | 1.2 | 1.1 | 1.1 | 1.0 | 0.9 | 1.0 | 1.0 | 1.3 | 1.1 | 1.4 |
| P61289_K12 | 1.4 | 0.7 | — | 0.9 | 1.9 | 0.9 | 0.8 | 3.5 | 0.9 | — |
| P61289_K237 | — | 0.8 | 1.4 | 1.0 | — | 0.9 | 0.7 | 2.2 | 0.9 | — |
| P61978_K405 | 1.4 | 0.9 | — | 0.9 | 20.0 | 0.8 | 0.9 | 1.0 | — | 1.3 |
| P62333_K72 | — | — | — | 0.9 | — | 1.1 | — | 1.2 | — | 1.9 |
| P62875_K67 | 1.0 | 0.9 | 4.1 | 0.9 | — | 0.9 | 1.0 | 2.6 | 1.0 | — |
| P68104_K84 | — | — | 0.7 | 20.0 | — | — | — | — | — | 0.7 |
| Q00765_K147 | — | — | — | — | — | 1.2 | — | — | — | — |
| Q01813_K688 | 1.9 | 3.3 | 20.0 | 1.0 | 4.1 | 1.0 | 1.0 | 4.9 | 1.0 | 3.9 |
| Q0VFZ6_K312 | — | 14.4 | — | — | — | — | — | — | — | — |
| Q12931_K699 | 1.8 | 0.9 | 1.7 | 0.9 | 1.4 | 0.9 | 0.9 | 1.3 | 1.0 | 1.0 |
| Q13011_K112 | 1.1 | 1.0 | 1.3 | 0.9 | 0.9 | 1.1 | 1.0 | 1.0 | 1.0 | 1.2 |
| Q13033_K755 | 3.7 | 1.0 | 1.3 | 0.8 | 0.9 | 0.9 | 1.2 | 1.3 | 0.9 | — |
| Q13148_K114 | — | — | 1.2 | 1.3 | — | 0.9 | — | — | — | — |
| Q13561_K175 | 1.7 | 0.8 | — | 0.9 | — | 0.8 | 0.9 | 1.3 | 0.9 | 0.6 |
| Q13617_K489 | 1.1 | 0.9 | 1.0 | 1.0 | 1.4 | 20.0 | 20.0 | 1.0 | — | 1.2 |
| Q13630_K108 | — | — | — | 2.9 | 0.9 | — | 0.9 | 1.3 | 1.0 | — |
| Q14204_K1649 | — | 1.0 | 1.2 | — | 1.0 | 1.0 | 0.8 | 1.0 | 0.8 | 0.9 |
| Q14789_K103 | 0.9 | 0.9 | 0.9 | 0.9 | 0.9 | 0.9 | 0.9 | 1.1 | 0.9 | 1.0 |
| Q14914_K194 | — | — | 1.7 | — | — | — | — | — | — | — |
| Q14CX7_K575 | — | 0.9 | 1.5 | 0.9 | — | 1.0 | 0.8 | — | — | — |
| Q15041_K188 | — | 1.4 | 1.6 | 1.8 | — | 1.7 | — | 1.3 | 1.1 | 1.4 |
| Q15233_K467 | — | — | — | 0.8 | — | — | 4.1 | — | 2.5 | 3.1 |
| Q16864_K6 | — | — | 1.0 | 1.0 | 1.0 | 0.9 | 0.9 | 1.0 | 1.0 | 20.0 |
| Q2M389_K302 | — | 1.4 | 1.2 | 0.9 | — | 1.2 | 0.9 | 1.5 | — | — |
| Q5TFE4_K123 | — | — | 20.0 | 1.8 | — | — | — | — | 0.6 | — |
| Q6NUQ1_K771 | 1.3 | 1.7 | 0.9 | 1.3 | 1.2 | 1.5 | 1.1 | 0.8 | 1.1 | 1.0 |
| Q6NZI2_K326 | — | — | — | 20.0 | — | 0.8 | 20.0 | — | — | — |
| Q7L0Y3_K325 | 1.0 | 1.0 | — | 1.0 | 0.8 | 0.9 | 0.9 | 0.9 | 1.0 | 1.0 |
| Q8N163_K112 | 1.0 | — | 1.1 | 1.1 | — | 1.1 | 1.5 | 1.3 | 1.1 | 1.0 |
| Q8N163_K97 | 1.3 | 0.8 | 0.9 | 1.0 | — | 0.9 | 1.0 | 1.0 | 1.0 | 0.9 |
| Q8TCA0_K43 | — | 1.0 | 1.1 | — | — | 0.9 | — | — | 1.3 | 1.2 |
| Q92600_K230 | 0.8 | 0.9 | 2.5 | 0.8 | 0.9 | 0.8 | 0.9 | 2.6 | 0.8 | 6.8 |
| Q969Y2_K492 | 1.6 | — | 2.9 | 1.2 | 1.1 | 1.2 | 0.6 | — | 0.9 | 1.5 |
| Q96AB3_K178 | 1.1 | 1.3 | 1.7 | 0.9 | 0.9 | 0.9 | 0.9 | 1.9 | 1.0 | 1.1 |
| Q96C01_K99 | 1.2 | — | 0.8 | 1.0 | — | 0.8 | 0.8 | 0.9 | 0.9 | 1.1 |
| Q96EL2_K94 | 1.4 | 1.3 | 2.8 | 0.9 | 3.1 | 1.2 | — | 1.3 | 1.5 | 1.1 |
| Q96HE7_K413 | — | 2.2 | — | 2.4 | — | — | — | 1.7 | — | 2.0 |
| Q96ST3_K155 | 0.8 | 1.6 | 1.4 | 1.1 | 1.2 | 1.0 | 1.1 | 1.3 | 0.9 | 1.2 |
| Q9BRQ3_K60 | — | 1.1 | — | 1.0 | — | 0.9 | 0.9 | 20.0 | 0.8 | — |
| Q9BSH5_K15 | 4.1 | 7.0 | 10.4 | 4.3 | 1.2 | 1.3 | 0.9 | 2.8 | 0.9 | 3.1 |
| Q9BYT8_K253 | — | 1.1 | — | 0.9 | 0.8 | 1.1 | 0.9 | 1.3 | 0.8 | 0.8 |
| Q9GZQ8_K51 | 1.0 | 1.0 | 0.8 | 0.9 | 0.8 | 0.9 | 1.0 | 0.9 | 0.9 | — |
| Q9GZV4_K47 | — | — | — | — | 1.8 | — | — | — | — | — |
| Q9H3P7_K117 | 1.1 | 1.3 | 1.4 | 0.9 | 1.0 | 0.9 | 0.9 | 1.2 | 1.0 | 1.2 |
| Q9H4M9_K511 | — | — | — | — | — | — | — | — | — | 20.0 |
| Q9H6D7_K271 | — | — | — | 0.7 | — | — | 0.7 | — | — | — |
| Q9H9B4_K170 | — | — | — | — | 1.9 | — | — | 2.5 | — | — |
| Q9HC38_K305 | 20.0 | 0.4 | — | 0.6 | 20.0 | 0.8 | 0.8 | 20.0 | 0.9 | 0.7 |
| Q9NQC3_K1104 | — | 6.0 | — | 3.4 | — | — | 2.1 | 4.3 | 1.2 | 1.4 |
| Q9NTK5_K248 | 0.9 | 1.0 | 0.8 | 0.9 | 0.9 | 0.9 | 0.9 | 1.0 | 1.0 | 1.0 |
| Q9NUJ1_K185 | 1.5 | 1.3 | 1.4 | 1.2 | 3.3 | 1.0 | 0.9 | 3.3 | 1.0 | 1.3 |
| Q9NVS9_K100 | 0.9 | 0.9 | 1.0 | 0.9 | 0.9 | 0.9 | 0.9 | 1.0 | 0.9 | 1.0 |
| Q9NZ08_K685 | 1.7 | — | 1.1 | 1.0 | 1.0 | 1.0 | 1.1 | 1.3 | 1.0 | 1.1 |
| Q9UBP0_K180 | — | — | — | 1.1 | 2.1 | 1.4 | — | 1.4 | 1.0 | 2.7 |
| Q9UBT2_K409 | — | — | 3.6 | — | — | — | — | — | — | — |
| Q9UHY7_K111 | 0.9 | 1.0 | 0.9 | 0.9 | 1.1 | 0.8 | 0.7 | 1.1 | — | 0.9 |
| Q9UKV3_K103 | — | — | — | — | — | — | — | — | — | — |
| Q9Y4K4_K49 | — | — | — | — | — | — | — | — | — | — |
| Q9Y5K5_K323 | 0.9 | 1.0 | 0.8 | 0.9 | 0.9 | 0.9 | 1.0 | 1.0 | 1.0 | 1.0 |
| Q9Y5X2_K316 | — | — | 3.6 | — | — | 1.4 | 2.0 | — | — | — |

34¹ - 50 uM_in vitro_sol_231
35² - 50 uM_in vitro_sol_231
36³ - 50 uM_231_sol_invitro
37⁴ - 50 uM_in vitro_sol_231
38⁵ - 50 uM_in vitro_sol_231
39⁶ - 50 uM_in vitro_sol_231
40⁷ - 50 uM_in vitro_sol_231
41⁸ - 50 uM_in vitro_sol_231
42⁹ - 50 uM_in vitro_sol_231
43¹⁰ - 50 uM_in vitro_sol_231

TABLE 1D

| | | | | | | | | | Possible | |
|---|---|---|---|---|---|---|---|---|---|---|
| Identifier | 44 | 45 | 46 | 47 | 48 | 49 | 50 | 51 | other lysine | protein class |
| A0AVT1_K409 | — | 6.3 | — | — | — | — | — | — | | Enzyme |
| O14879_K148 | 2.2 | 2.7 | 2.2 | — | 0.7 | — | 1.4 | — | | Gene Expression, |
| | | | | | | | | | | Replication, Nucleic |
| | | | | | | | | | | Acid Binding |
| O43399_K90 | 1.1 | 1.5 | 1.4 | 2.0 | 1.8 | 2.9 | 2.2 | 1.0 | | Gene Expression, |
| | | | | | | | | | | Replication, Nucleic |
| | | | | | | | | | | Acid Binding |
| O43747_K214 | 0.9 | 1.2 | 1.0 | 0.8 | 0.8 | 1.0 | 1.0 | 0.9 | | Transporter, Channel, |
| | | | | | | | | | | Receptor |
| O43837_K207 | — | — | — | — | — | — | 3.1 | — | | Enzyme |
| O60664_K140 | — | 2.1 | — | — | 1.3 | 3.0 | 1.8 | — | | Scaffolding, Modulator, |
| | | | | | | | | | | Adaptor |
| O60664_K257 | 1.9 | 2.5 | 0.6 | — | — | — | — | 1.0 | | Scaffolding, Modulator, |
| | | | | | | | | | | Adaptor |
| O75323_K75 | 1.1 | 5.0 | 0.9 | 1.0 | 0.9 | 1.7 | 1.3 | 0.9 | | Scaffolding, Modulator, |
| | | | | | | | | | | Adaptor |
| O75821_K280 | 0.9 | 0.9 | 0.8 | — | — | — | — | — | | Gene Expression, |
| | | | | | | | | | | Replication, Nucleic |
| | | | | | | | | | | Acid Binding |
| O95197_K1022 | — | — | — | — | — | — | — | — | | Scaffolding, Modulator, |
| | | | | | | | | | | Adaptor |
| O95628_K134 | 2.0 | 2.8 | 5.2 | 1.5 | — | 1.4 | 1.7 | 0.9 | | Enzyme |
| P00367_K480 | 1.2 | 2.8 | 1.5 | 2.0 | 1.5 | 2.0 | 1.3 | 1.5 | P00367_K477 | Enzyme |
| P02545_K135 | 0.9 | 0.9 | 0.7 | 1.2 | 1.0 | 1.1 | 1.3 | 1.0 | | Scaffolding, Modulator, |
| | | | | | | | | | | Adaptor |
| P02766_K35 | 20.0 | 2.5 | 2.3 | 1.6 | — | — | 2.1 | — | | Scaffolding, Modulator, |
| | | | | | | | | | | Adaptor |
| P04179_K68 | 1.6 | 0.9 | 0.9 | — | — | 3.3 | — | 0.9 | | Enzyme |
| P04181_K66 | — | — | — | — | — | — | 9.9 | — | | Enzyme |
| P05141_K23 | 1.1 | 1.2 | 0.9 | 1.8 | 1.2 | — | 12.6 | — | | Scaffolding, Modulator, |
| | | | | | | | | | | Adaptor |
| P07195_K244 | 1.1 | — | 0.8 | 1.0 | — | — | 1.1 | — | | Enzyme |
| P07195_K82 | 1.0 | — | 0.7 | — | — | — | 0.9 | — | | Enzyme |
| P07954_K311 | — | 1.8 | — | 1.4 | — | — | — | 1.0 | | Enzyme |
| P08237_K678 | 1.9 | 4.8 | 1.3 | 0.8 | 0.9 | 5.4 | 20.0 | 0.8 | | Enzyme |
| P0CG30_K53 | 0.6 | 0.5 | 1.8 | 0.6 | 0.9 | 3.1 | 1.4 | 0.7 | | Enzyme |
| P11413_K171 | 1.3 | 1.1 | 0.9 | 1.0 | 0.9 | 1.2 | 1.1 | 0.9 | | Enzyme |
| P11586_K760 | — | — | — | — | — | — | 0.6 | — | | Enzyme |
| P12956_K351 | 1.1 | 1.9 | 2.5 | 1.4 | 1.5 | 20.0 | 6.0 | 0.8 | | Enzyme |
| P13639_K235 | 1.0 | 1.0 | 1.3 | 1.1 | 1.2 | 6.2 | 7.5 | 0.9 | | Gene Expression, |
| | | | | | | | | | | Replication, Nucleic |
| | | | | | | | | | | Acid Binding |
| P13639_K318 | 1.1 | 1.0 | 1.1 | 1.1 | 1.1 | 2.0 | 1.8 | 0.9 | | Gene Expression, |
| | | | | | | | | | | Replication, Nucleic |
| | | | | | | | | | | Acid Binding |
| P13726_K279 | 1.1 | 4.2 | — | — | — | — | 1.0 | — | | Scaffolding, Modulator, |
| | | | | | | | | | | Adaptor |
| P13804_K139 | 0.9 | 1.0 | 0.9 | 1.1 | 0.9 | — | 1.1 | 1.0 | | Enzyme |
| P16930_K241 | 2.5 | 1.3 | 8.1 | 1.2 | 2.3 | 1.3 | 1.5 | 0.9 | | Enzyme |
| P17405_K118 | 0.8 | 1.1 | 0.7 | — | — | 8.4 | 1.3 | 0.9 | | Enzyme |
| P17858_K315 | 0.8 | 1.8 | 0.6 | 1.0 | 0.7 | — | — | — | | Enzyme |
| P17858_K677 | 1.7 | 2.9 | 1.4 | 1.0 | 0.9 | — | 20.0 | 0.9 | P17858_K681 | Enzyme |
| P17858_K715 | 1.8 | 10.9 | 0.8 | 1.0 | 1.0 | 1.8 | 2.9 | 0.9 | P17858_K714 | Enzyme |
| P19367_K510 | 1.2 | 5.3 | 1.0 | 0.9 | 0.8 | 1.1 | 1.0 | 1.0 | | Enzyme |
| P20248_K54 | — | — | — | — | — | — | — | — | | Scaffolding, Modulator, |
| | | | | | | | | | | Adaptor |
| P20839_K208 | — | 1.5 | — | 1.2 | — | 4.4 | 20.0 | — | | Enzyme |
| P22830_K304 | 1.1 | 1.4 | 0.9 | 2.2 | 1.1 | 5.3 | 2.3 | 0.8 | | Enzyme |
| P23381_K256 | 1.1 | 1.1 | 1.6 | — | — | 1.0 | 1.1 | — | | Enzyme |
| P23919_K118 | — | — | — | 0.9 | 0.6 | — | — | — | | Enzyme |
| P24941_K33 | 1.1 | 4.6 | 0.9 | — | — | — | 2.3 | — | | Enzyme |
| P26358_K45 | 0.7 | 1.0 | 2.4 | — | 1.1 | 1.3 | — | — | | Enzyme |
| P26641_K227 | 0.8 | 0.6 | 0.6 | — | — | — | 1.2 | 0.9 | | Enzyme |
| P27635_K121 | 0.6 | 1.8 | 0.8 | 1.1 | 0.8 | 1.8 | 1.0 | — | | Gene Expression, |
| | | | | | | | | | | Replication, Nucleic |
| | | | | | | | | | | Acid Binding |
| P32969_K82 | 14.5 | 1.0 | 0.5 | — | — | — | — | — | | Gene Expression, |
| | | | | | | | | | | Replication, Nucleic |
| | | | | | | | | | | Acid Binding |
| P36551_K404 | 1.0 | 1.2 | 1.0 | 1.5 | 1.0 | 2.5 | 1.8 | 0.9 | | Enzyme |
| P42330_K270 | 0.5 | 0.8 | — | 2.5 | — | 0.8 | 0.7 | 0.8 | P52895_K270, | Enzyme |
| | | | | | | | | | P17516_K270, | |
| | | | | | | | | | Q04828_K270 | |
| P42345_K2066 | 1.7 | 1.1 | 1.5 | 1.1 | 1.0 | 1.0 | 1.4 | 0.8 | | Enzyme |
| P46734_K93 | 0.8 | 0.9 | 1.3 | 1.1 | 1.1 | 6.8 | 1.8 | 1.0 | | Enzyme |
| P46783_K139 | 0.6 | — | — | — | 0.8 | 20.0 | 1.1 | — | | Gene Expression, |
| | | | | | | | | | | Replication, Nucleic |
| | | | | | | | | | | Acid Binding |
| P50583_K89 | 0.9 | 1.7 | 1.1 | 1.0 | 1.0 | 20.0 | 20.0 | 0.9 | P50583_K87 | Enzyme |
| P51580_K32 | 1.4 | 2.3 | 20.0 | 9.5 | 4.4 | — | 11.7 | 0.8 | | Enzyme |
| P52292_K459 | 1.6 | 1.7 | 1.4 | 1.2 | 1.1 | 1.1 | 1.6 | 0.9 | | Transporter, Channel, |
| | | | | | | | | | | Receptor |
| P52594_K134 | 1.5 | 20.0 | 1.0 | — | 1.0 | 0.8 | 1.0 | 0.9 | | Gene Expression, |
| | | | | | | | | | | Replication, Nucleic |
| | | | | | | | | | | Acid Binding |
| P52815_K87 | 1.1 | 1.8 | 0.6 | 1.1 | 0.9 | — | — | 0.9 | B4DLN1_K87, | Gene Expression, |
| | | | | | | | | | P52815_K91 | Replication, Nucleic |
| | | | | | | | | | | Acid Binding |
| P55263_K88 | 0.5 | 1.3 | 0.8 | 0.8 | 0.9 | 1.2 | 1.0 | 0.8 | | Enzyme |
| P55786_K712 | 1.0 | 1.3 | 1.3 | 1.0 | 1.0 | 1.3 | 1.6 | 1.0 | | Enzyme |
| P60520_K46 | 0.9 | 1.0 | — | 1.0 | 0.9 | 1.9 | 20.0 | 1.0 | | Scaffolding, Modulator, |
| | | | | | | | | | | Adaptor |
| P61011_K81 | 1.0 | 1.0 | 0.9 | 0.9 | 1.1 | 1.0 | — | 0.7 | | Gene Expression, |
| | | | | | | | | | | Replication, Nucleic |
| | | | | | | | | | | Acid Binding |
| P61221_K191 | 1.1 | 1.1 | 20.0 | 1.7 | 1.0 | 20.0 | 14.1 | 1.0 | | Scaffolding, Modulator, |
| | | | | | | | | | | Adaptor |
| P61221_K478 | 0.9 | 1.5 | 11.4 | 1.4 | 1.1 | 1.0 | 2.1 | 0.9 | | Scaffolding, Modulator, |
| | | | | | | | | | | Adaptor |
| P61289_K12 | — | 2.0 | 0.7 | 1.1 | — | 0.8 | 0.7 | — | | Scaffolding, Modulator, |
| | | | | | | | | | | Adaptor |
| P61289_K237 | — | 1.6 | 0.9 | 0.9 | — | 0.8 | 0.9 | 0.7 | | Scaffolding, Modulator, |
| | | | | | | | | | | Adaptor |
| P61978_K405 | 1.0 | 1.1 | 1.1 | — | — | 1.9 | 1.3 | 1.2 | | Gene Expression, |
| | | | | | | | | | | Replication, Nucleic |
| | | | | | | | | | | Acid Binding |
| P62333_K72 | — | — | 0.9 | — | — | — | 1.3 | — | | Gene Expression, |
| | | | | | | | | | | Replication, Nucleic |
| | | | | | | | | | | Acid Binding |
| P62875_K67 | 1.0 | 2.8 | 1.0 | 1.0 | 0.9 | — | 1.0 | 1.0 | | Enzyme |
| P68104_K84 | — | — | — | — | — | 1.2 | 1.3 | — | | Gene Expression, |
| | | | | | | | | | | Replication, Nucleic |
| | | | | | | | | | | Acid Binding |
| Q00765_K147 | — | 2.7 | — | — | — | — | — | — | | No classification |
| Q01813_K688 | 4.2 | 20.0 | 1.7 | 1.0 | 1.4 | 9.6 | 18.2 | 0.9 | | Enzyme |
| Q0VFZ6_K312 | — | — | — | — | — | — | — | — | | No classification |
| Q12931_K699 | 0.9 | 1.4 | 0.9 | 1.1 | 1.0 | 1.2 | 1.1 | 1.2 | | Gene Expression, |
| | | | | | | | | | | Replication, Nucleic |
| | | | | | | | | | | Acid Binding |
| Q13011_K112 | 0.9 | 1.0 | 0.8 | 1.0 | 1.5 | 1.1 | 1.3 | 1.0 | | Enzyme |
| Q13033_K755 | 1.0 | 1.3 | 11.4 | 0.9 | 1.2 | 1.8 | 1.2 | 0.9 | | Scaffolding, Modulator, |
| | | | | | | | | | | Adaptor |
| Q13148_K114 | 20.0 | 20.0 | — | — | — | 20.0 | 1.2 | — | | Gene Expression, |
| | | | | | | | | | | Replication, Nucleic |
| | | | | | | | | | | Acid Binding |
| Q13561_K175 | 0.8 | 1.6 | 0.8 | 1.0 | 0.7 | 1.5 | 1.3 | 0.9 | | Scaffolding, Modulator, |
| | | | | | | | | | | Adaptor |
| Q13617_K489 | 0.8 | 1.2 | 1.0 | 0.8 | 0.8 | 1.1 | 1.0 | 1.1 | | Scaffolding, Modulator, |
| | | | | | | | | | | Adaptor |
| Q13630_K108 | — | 20.0 | 2.7 | — | — | — | 1.0 | — | | Enzyme |
| Q14204_K1649 | 1.3 | 1.0 | 1.3 | 0.9 | — | — | 1.3 | 0.7 | | Gene Expression, |
| | | | | | | | | | | Replication, Nucleic |
| | | | | | | | | | | Acid Binding |
| Q14789_K103 | 0.9 | 1.0 | 1.2 | 1.0 | 1.1 | 1.8 | 1.4 | 0.9 | | Gene Expression, |
| | | | | | | | | | | Replication, Nucleic |
| | | | | | | | | | | Acid Binding |
| Q14914_K194 | — | 5.5 | — | — | — | 1.3 | 1.2 | — | | Enzyme |
| Q14CX7_K575 | 0.8 | 1.2 | — | — | 1.1 | 1.1 | — | 1.0 | | Scaffolding, Modulator, |
| | | | | | | | | | | Adaptor |
| Q15041_K188 | 1.1 | 2.2 | 1.2 | 3.5 | 3.5 | — | 3.4 | — | | Scaffolding, Modulator, |
| | | | | | | | | | | Adaptor |
| Q15233_K467 | 0.9 | — | 0.4 | — | — | 4.9 | — | — | | Gene Expression, |
| | | | | | | | | | | Replication, Nucleic |
| | | | | | | | | | | Acid Binding |
| Q16864_K6 | 1.2 | 1.0 | 20.0 | — | — | 1.3 | 1.4 | — | | Transporter, Channel, |
| | | | | | | | | | | Receptor |
| Q2M389_K302 | 1.3 | 1.2 | — | — | — | — | 5.3 | 1.5 | | No classification |
| Q5TFE4_K123 | — | 2.1 | 20.0 | — | — | 1.3 | 1.1 | — | | Enzyme |
| Q6NUQ1_K771 | 1.7 | 1.0 | 20.0 | 1.2 | 4.8 | 1.1 | 1.1 | 0.9 | | Scaffolding, Modulator, |
| | | | | | | | | | | Adaptor |
| Q6NZI2_K326 | — | 0.9 | — | — | — | — | — | — | | Gene Expression, |
| | | | | | | | | | | Replication, Nucleic |
| | | | | | | | | | | Acid Binding |
| Q7L0Y3_K325 | 1.3 | 1.0 | 1.0 | 1.1 | — | 1.2 | 6.2 | 0.9 | | Enzyme |
| Q8N163_K112 | 1.0 | 1.2 | 1.1 | 1.5 | 1.0 | 20.0 | 4.7 | 0.8 | | Gene Expression, |
| | | | | | | | | | | Replication, Nucleic |
| | | | | | | | | | | Acid Binding |
| Q8N163_K97 | 0.8 | 0.8 | 0.9 | 0.9 | 0.6 | 9.4 | 3.9 | 0.8 | | Gene Expression, |
| | | | | | | | | | | Replication, Nucleic |
| | | | | | | | | | | Acid Binding |
| Q8TCA0_K43 | 1.1 | 0.8 | 1.3 | — | — | 14.0 | 1.3 | — | | Scaffolding, Modulator, |
| | | | | | | | | | | Adaptor |
| Q92600_K230 | 1.0 | 1.7 | 1.1 | 0.8 | — | 1.1 | 1.0 | 0.9 | | Gene Expression, |
| | | | | | | | | | | Replication, Nucleic |
| | | | | | | | | | | Acid Binding |
| Q969Y2_K492 | 0.9 | 1.4 | 1.9 | 1.7 | 1.4 | — | 0.9 | — | | No classification |
| Q96AB3_K178 | 1.3 | 1.8 | 1.5 | 1.2 | 0.9 | 1.1 | 1.1 | 0.9 | | No classification |
| Q96C01_K99 | — | 1.0 | 0.6 | 0.7 | 5.5 | 1.3 | — | — | | No classification |
| Q96EL2_K94 | 1.1 | — | 1.0 | — | 1.0 | 3.0 | 2.2 | 0.9 | | Gene Expression, |
| | | | | | | | | | | Replication, Nucleic |
| | | | | | | | | | | Acid Binding |
| Q96HE7_K413 | 1.9 | — | 6.5 | — | — | — | 1.4 | — | Q86YB8_K412 | Enzyme |
| Q96ST3_K155 | 1.5 | 1.5 | 4.4 | 1.8 | 20.0 | — | 3.5 | 1.1 | Q96ST3_K152 | Gene Expression, |
| | | | | | | | | | | Replication, Nucleic |
| | | | | | | | | | | Acid Binding |
| Q9BRQ3_K60 | — | 1.7 | 0.9 | 1.1 | — | — | 1.0 | — | | Enzyme |
| Q9BSH5_K15 | 20.0 | 3.4 | 1.4 | 0.9 | 1.0 | 3.5 | 2.8 | 0.8 | | Enzyme |
| Q9BYT8_K253 | — | 1.3 | 1.5 | — | — | — | 1.2 | — | | Enzyme |
| Q9GZQ8_K51 | 0.9 | 1.0 | — | 0.9 | 0.8 | 1.6 | 4.6 | — | | Scaffolding, Modulator, |
| | | | | | | | | | | Adaptor |
| Q9GZV4_K47 | — | — | — | — | — | — | — | — | | Gene Expression, |
| | | | | | | | | | | Replication, Nucleic |
| | | | | | | | | | | Acid Binding |
| Q9H3P7_K117 | 0.8 | 1.1 | 7.7 | 1.0 | 0.9 | 13.9 | 14.9 | 0.9 | | Scaffolding, Modulator, |
| | | | | | | | | | | Adaptor |
| Q9H4M9 K511 | — | — | — | 20.0 | — | — | — | — | | Scaffolding, Modulator, |
| | | | | | | | | | | Adaptor |
| Q9H6D7_K271 | 0.9 | 1.1 | — | — | — | — | 4.3 | — | | Gene Expression, |
| | | | | | | | | | | Replication, Nucleic |
| | | | | | | | | | | Acid Binding |
| Q9H9B4_K170 | 0.9 | 2.1 | 0.9 | — | — | — | 1.9 | — | | Transporter, Channel, |
| | | | | | | | | | | Receptor |
| Q9HC38_K305 | 0.5 | 0.7 | — | — | — | 1.2 | 1.1 | 0.9 | | No classification |
| Q9NQC3_K1104 | 2.6 | — | 2.5 | 3.8 | — | — | 1.4 | 0.7 | | Gene Expression, |
| | | | | | | | | | | Replication, Nucleic |
| | | | | | | | | | | Acid Binding |
| Q9NTK5_K248 | 1.1 | 1.0 | 1.2 | 1.2 | 1.0 | 7.0 | 2.1 | 0.9 | | Gene Expression, |
| | | | | | | | | | | Replication, Nucleic |
| | | | | | | | | | | Acid Binding |
| Q9NUJ1_K185 | 1.2 | 1.2 | 1.2 | 1.8 | 2.7 | 1.5 | 1.3 | 1.0 | | Enzyme |
| Q9NVS9_K100 | 0.9 | 1.0 | 1.0 | 0.9 | 0.9 | 3.3 | 1.1 | 0.9 | | Enzyme |
| Q9NZ08_K685 | 1.6 | 1.3 | 2.1 | 1.3 | 1.5 | 1.6 | 20.0 | 0.9 | | Enzyme |
| Q9UBP0_K180 | — | 1.7 | 4.4 | — | — | — | 2.0 | — | | Scaffolding, Modulator, |
| | | | | | | | | | | Adaptor |
| Q9UBT2_K409 | — | — | — | — | — | — | — | — | | Enzyme |
| Q9UHY7_K111 | 0.9 | 1.1 | 1.1 | 7.8 | 0.9 | 20.0 | 15.1 | 0.9 | | Enzyme |
| Q9UKV3_K103 | 1.0 | — | 16.6 | — | — | — | — | — | | Gene Expression, |
| | | | | | | | | | | Replication, Nucleic |
| | | | | | | | | | | Acid Binding |
| Q9Y4K4_K49 | — | — | — | — | — | — | — | — | | Enzyme |
| Q9Y5K5_K323 | 0.9 | 1.5 | 1.0 | 1.4 | 0.9 | 9.0 | 2.2 | 1.0 | | Enzyme |
| Q9Y5X2_K316 | — | — | 2.7 | — | — | — | — | 1.2 | | Scaffolding, Modulator, |
| | | | | | | | | | | Adaptor |

44¹ - 50 uM_in vitro_sol_231
45² - 50 uM_231_sol_invitro
46³ - 50 uM_231_sol_in vitro
47⁴ - 50 uM_231_sol_in vitro
48⁵ - 50 uM_in vitro_sol_231
49⁶ - 100 uM_231_sol_in vitro
50⁷ - 100 uM_231_sol_in vitro
51⁸ - 50 uM_231_sol_in vitro

Table 2 (48054-708-201Table2.txt) illustrates ractivity ratio of liganded lysines identified in the isoTOP-ABPP experiments described above. Table 2 is submitted as a computer readable text file in ASCII format and is hereby incorporated in its entirety by reference herein.

Table 3 (48054-708-201Table3.txt) illustrates a list of unliganded lysines and their reactivity profiles with the fragment eletrophile library from isoTOP-ABPP experiments described above. Table 3 is submitted as a computer readable text file in ASCII format and is hereby incorporated in its entirety by reference herein.

Table 4 illustrates exemplary lysine reactive ratios.

| | | | | | | | | | | number | KR or |
|---|---|---|---|---|---|---|---|---|---|---|---|
| | | probe | | | | | | | | of | KA |
| protein | lysine | 1 | 16 | 17 | 18 | 10 | 12 | 13 | 6 | lysines | mutant |
| ADK | K88 | PD | PD | ND | PD | HD | HD | HD | NP | 34 | KR |
| CARM1 | K241 | HD | NL | ND | PD | NP | ND | ND | ND | 19 | KR |
| FAH | K241 | ND | ND | ND | PD | NP | PD | NP | NP | 19 | KR |
| G6PD | K171 | ND | ND | ND | NL | NP | ND | ND | ND | 29 | KR |
| GSTO1 | K57 | PD | ND | ND | ND | NP | ND | ND | ND | 23 | KR |
| HDHD3 | K15 | HD | HD | PD | HD | HD | HD | HD | HD | 5 | KR |
| HK1 | K510 | PD | ND | ND | ND | NP | ND | ND | HD | 59 | KR |
| PFKL | K676 | PD | PD | ND | PD | PD | PD | PD | NP | 37 | KA |
| PFKM | K678 | HD | PD | ND | PD | PD | PD | PD | ND | 45 | KR |
| PFKP | K688 | HD | HD | ND | PD | HD | HD | HD | HD | 42 | KR |
| PMVK | K48 | HD | HD | PD | HD | ND | ND | ND | ND | 8 | KR |
| PNPO | K100 | HD | HD | HD | PD | NP | PD | ND | PD | 14 | KR |
| Sin3a | K155 | PD | NL | HD | HD | HD | HD | HD | HD | 87 | KR |
| TPMT | K32 | HD | ND | PD | HD | NP | ND | ND | ND | 21 | KR |
| TTR | K35 | HD | NL | HD | HD | HD | HD | HD | HD | 8 | KA |

NP: experiment was not done
NL: labelling of the WT protein was not detected by gel (intensity for WT less than 2x background)
ND: the WT did not label more than the mutant (<2x difference quantified)
PD: the WT labeled more than the mutant (>2x difference quantified)
HD: the WT labeled more than the mutant (>4x difference quantified)
Bold: chaser that has been used for follow-up experiments

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of identifying a reactive lysine of a protein, comprising:
a) providing a protein sample comprising isolated proteins, living cells, or a cell lysate;
b) contacting the protein sample with a probe compound of Formula (I) at a first concentration for a time sufficient for the probe compound to react with the reactive lysine of the protein sample; and
c) analyzing the proteins of the protein sample to identify the reactive lysine that bound with the probe compound at the first concentration;
wherein the probe compound has a structure represented by Formula (I): 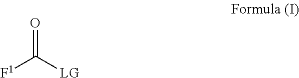
wherein,
F¹ is a small molecule fragment moiety comprising an alkyne moiety, a fluorophore moiety, a labeling group, or a combination thereof; and
LG is a leaving group moiety.

2. The method of claim 1, wherein F¹ comprises an alkyne moiety.

3. The method of claim 1, wherein F¹ comprises a fluorophore moiety.

4. The method of claim 1, wherein LG comprises a succinimide moiety or a phenyl moiety.

5. The method of claim 4, wherein LG comprises the phenyl moiety.

6. The method of claim 5, wherein the phenyl moiety comprises one or more substituents selected from the group consisting of halogen, C₁-C₆fluoroalkyl, —CN, —NO₂, —S(═O)R¹, —S(═O)₂R¹, —S(═O)₂OM, —N(R¹)S(═O)₂R¹, —S(═O)₂NR¹R², —C(═O)R¹, —C(═O)OM, —OC(═O)R¹, —C(═O)OR², —OC(═O)OR², —C(═O)NR¹R², —OC(═O)NR¹R², —NR¹C(═O)NR¹R², and —NR¹C(═O)R¹;
each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl;
R² is independently selected from the group consisting of H, D, C₁-C₆alkyl, C₁-C₆fluoroalkyl, C₁-C₆heteroalkyl, and a substituted or unsubstituted aryl;
or R¹ and R⁶ are taken together with the intervening atoms joining R⁵ and R⁶ to form a 5- or 6-membered ring; and
M is Li, Na, K, or —N(R²)₄.

7. The method of claim 1, wherein the probe compound has a structure selected from:

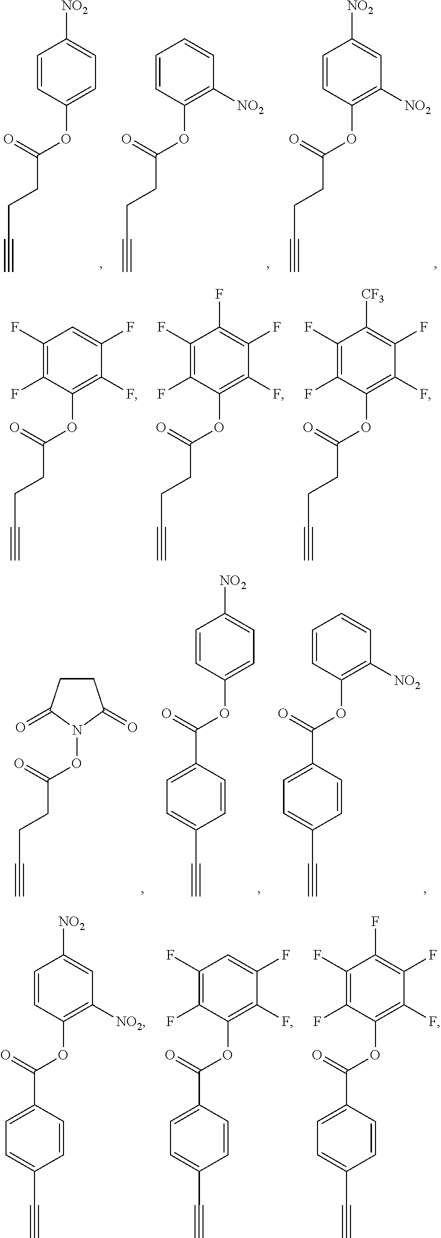

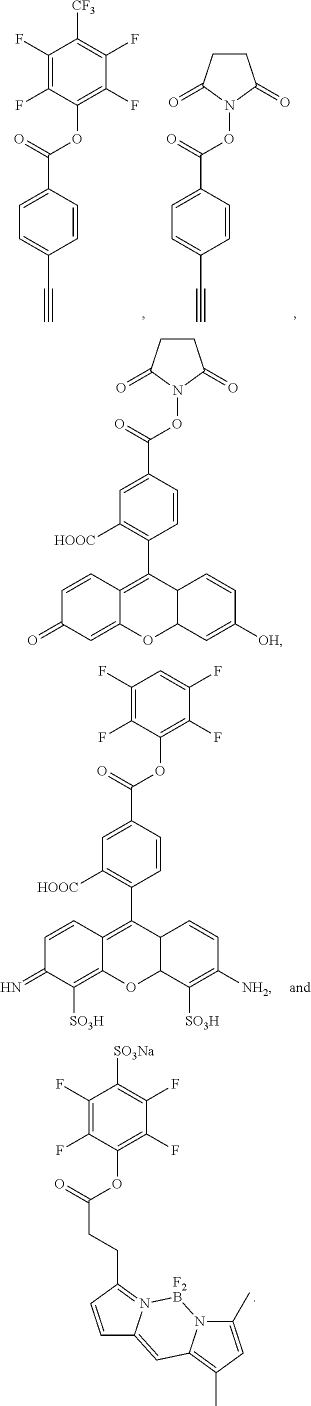

8. The method of claim 1, wherein the analyzing of step (c) further comprises tagging at least one lysine-containing protein-ligand complex of step (b) to generate a tagged lysine-containing protein-ligand complex.

9. The method of claim 8, wherein the analyzing of step (c) further comprises isolating the tagged lysine-containing protein-ligand complex.

10. The method of claim 8, wherein the tagging comprises attaching a biotin moiety.

11. The method of claim 10, wherein the biotin moiety comprises biotin or a biotin derivative.

12. The method of claim 11, wherein the biotin derivative comprises desthiobiotin, biotin alkyne or biotin azide.

13. The method of claim 10, wherein the biotin moiety comprises desthiobiotin.

14. The method of claim 1, further comprising:
a) providing an protein sample comprising isolated proteins, living cells, or a cell lysate and separating the protein sample into a first protein sample and a second protein sample;
b) contacting the first protein sample with a probe compound of Formula (I) at a first concentration for a time sufficient for the probe compound to react with a reactive lysine of the first protein sample, and contacting the second protein sample with the probe compound of Formula (I) at a second concentration for a sufficient time for the probe compound to react with a reactive lysine of the second protein sample;
c) tagging the proteins of the first protein sample and the second protein sample of step b) to generate tagged proteins; and
d) isolating the tagged proteins of the first protein sample and the second protein sample for analysis.

15. A method of identifying a reactive lysine of a protein, comprising:
a) providing a protein sample comprising isolated proteins, living cells, or a cell lysate and separating the protein sample into a first protein sample and a second protein sample;
b) contacting the first protein sample with a probe compound of Formula (I) at a first concentration for a time sufficient for the probe compound to react with a reactive lysine of the first protein sample, and contacting the second protein sample with the probe compound of Formula (I) at a second concentration for a sufficient time for the probe compound to react with a reactive lysine of the second protein sample;
c) analyzing the proteins of the first protein sample and the second protein samples of step b) to identify the reactive lysines that bound with the probe compound;
d) comparing the identity of the reactive lysines of step c) from the first protein sample at the first concentration of probe compound to the reactive lysines from the second protein sample at the second concentration of probe compound; and
e) based on step d), determining a reactive lysine of a protein;
wherein the probe compound has a structure represented by Formula (I): 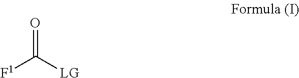
wherein,
F¹ is a small molecule fragment moiety comprising an alkyne moiety, a fluorophore moiety, a labeling group, or a combination thereof; and
LG is a leaving group moiety.

16-41. (canceled)

42. A modified lysine-containing protein comprising: a small molecule fragment moiety, covalently bonded to a lysine residue of a lysine-containing protein, wherein a covalent bond is formed by reaction with a non-naturally occurring small molecule probe having a structure of Formula (I): 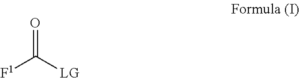
wherein,
F¹ is a small molecule fragment moiety comprising an alkyne moiety, a fluorophore moiety, a labeling group, or a combination thereof; and
LG is a leaving group moiety.

43-45. (canceled)

46. The modified lysine-containing protein of claim 42, wherein LG comprises a succinimide moiety or a phenyl moiety.

47. The modified lysine-containing protein of claim 46, wherein LG comprises the phenyl moiety.

48. The modified lysine-containing protein of claim 47, wherein the phenyl moiety comprises one or more substituents selected from the group consisting of halogen, C₁-C₆fluoroalkyl, —CN, —NO₂, —S(═O)R¹, —S(═O)₂R, —S(═O)₂OM, —N(R¹)S(═O)₂R¹, —S(═O)₂NR¹R², —C(═O)R¹, —C(═O)OM, —OC(═O)R¹, —C(═O)OR², —OC(═O)OR², —C(═O)NR¹R², —OC(═O)NR¹R², —NR¹C(═O)NR¹R², and —NR¹C(═O)R¹;
each R¹ is independently selected from the group consisting of H, D, —OR², C₁-C₆alkyl, C₁-C₆fluoroalkyl, C₁-C₆heteroalkyl, a substituted or unsubstituted C₃-C₆cycloalkyl, a substituted or unsubstituted C₂-C₆heterocycloalkyl, a substituted or unsubstituted aryl, and a substituted or unsubstituted heteroaryl;
R² is independently selected from the group consisting of H, D, C₁-C₆alkyl, C₁-C₆fluoroalkyl, C₁-C₆heteroalkyl, and a substituted or unsubstituted aryl;
or R¹ and R⁶ are taken together with the intervening atoms joining R⁵ and R⁶ to form a 5- or 6-membered ring; and
M is Li, Na, K, or —N(R²)₄.

49. The modified lysine-containing protein of claim 42, wherein the small molecule probe has a structure selected from:

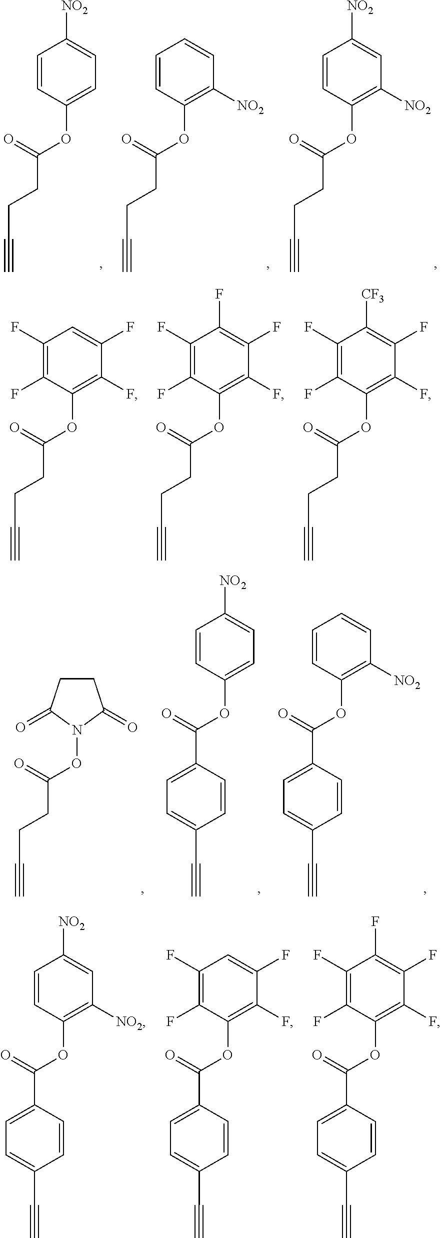

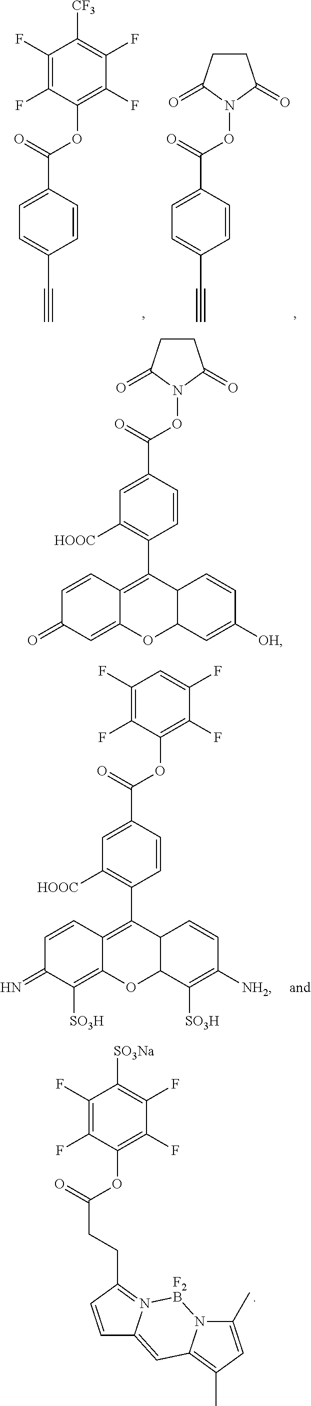

50-59. (canceled)
